(19) United States
(12) Patent Application Publication
(10) Pub. No.: US 2008/0227851 A1
(43) Pub. Date: Sep. 18, 2008

(54) LAULIMALIDE AND LAULIMALIDE ANALOGS

(76) Inventor: PAUL A. WENDER, Menlo Park, CA (US)

Correspondence Address: KING & SPALDING LLP, 1180 PEACHTREE STREET, ATLANTA, GA, 30309-3521, US

(21) Appl. No.: 12/046,632

(22) Filed: Mar. 12, 2008

Related U.S. Application Data
(60) Provisional application No. 60/906,625, filed on Mar. 12, 2007.
(60) Provisional application No. 60/983,992, filed on Oct. 31, 2007.

(51) Int. Cl. A61K 31/335; C07D 321/00; A61P 35/00; A61P 35/02

(52) U.S. Cl. 514/450; 549/349

(57) ABSTRACT

Novel laulimalide analogs, methods for the treatment of proliferative disease and processes for the synthesis of laulimalide and novel laulimalide analogs are described.

### CROSS-REFERENCE

This application claims the benefit of U.S. Provisional Application No. 60/906,625, fled Mar. 12, 2007, and U.S. Provisional Application No. 60/983,992, filed Oct. 31, 2007, which are incorporated herein by reference.

### BACKGROUND OF THE INVENTION

In 1988, research groups from the University of California, Santa Cruz and the University of Hawaii independently and simultaneously reported the isolation and characterization of two polyketides from the marine sponges Cacospongia mycofijiensis and Hyatella sp. (Quinoa et al., J. Org. Chem. 1988, 53, 3642-4; Corley et al., J. Org. Chem. 1988, 53, 3644-6). In the latter case, extracts from a nudibranch predator Chromodoris lochi found grazing on the sponge were also found to contain these compounds, which have since come to be known as laulimalide and isolaulimalide. The latter was found to be the product of acid-catalyzed decomposition of the former, proceeding to completion in four hours in a 0.01 N solution of HCI in acetone, presumably via nucleophilic attack of the C20 hydroxyl on the C16-C17 epoxide. In the years following these initial reports, the two compounds have been isolated from a number of other sources (Jefford et al., Tetrahedron Lett. 1996, 37, 159-62; Tanaka et al., Chem. Lett. 1996, 4, 255-6; Cutignano et al., Eur. J. Org. Chem. 2001, 775-8). The structure of laulimalide, originally assigned based on NMR and mass spectrometry data, was later confirmed along with its absolute configuration by X-ray crystal structure analysis (Jefford et al., Tetrahedron Lett. 1996, 37, 159-62).

The impetus for the isolation of these compounds from C. mycofijiensis was the extreme toxicity observed as a property of the liquid squeezed from a sample of the sponge, which killed tropical fish being held in an aquarium within 10 minutes. Further biological evaluation revealed that laulimalide is cytotoxic to KB cells in the low nanomolar range (IC₅₀=15 ng/mL) (Corley et al., J. Org. Chem. 1988, 53, 3644-6). Isolaulimalide was found to be much less cytotoxic (IC₅₀>200 ng/mL).

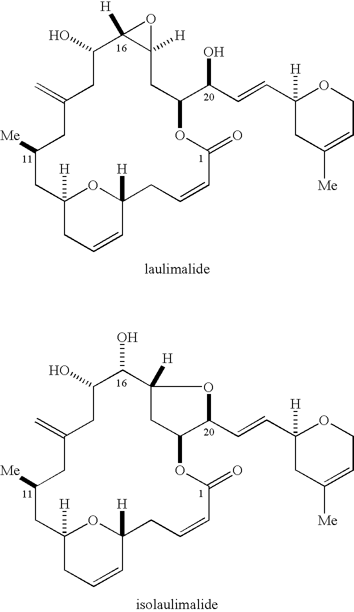

The laulimalides were ultimately found to exhibit cell cycle inhibition activity similar to other well-known therapeutics. The taxane anti-cancer drugs paclitaxel (Taxol) and docetaxel (Taxotere) initiate apoptosis of transformed cells through the stabilization of microtubules (Schiff et al., Proc. Natl. Acad. Sci. USA 1980, 77, 1561-65). Microtubules are dynamic structures that play a key role in many cellular processes and interruption of their normal assembly or disassembly has proven to be an effective strategy for cancer chemotherapy (Wilson et al., Chem. Biol. 1995, 2, 569-73). They are critical to cell division, since they are the major component of the mitotic spindle. The taxanes disrupt mitosis, eventually causing the initiation of apoptosis, by causing the abnormal formation of this structure. Given their significant clinical success, a vigorous search for new small molecules that share the taxanes' mechanism of action is ongoing. A major goal of this effort has been to identify compounds that are effective against multi-drug resistant (MDR) cell lines, including those that over-express the drug-efflux pump P-glycoprotein (P-gp), for which Taxol is a substrate (Cowden et al., Nature 1997, 387, 238-9).

In 1999, it was reported that laulimalide and isolaulimalide demonstrate remarkable Taxol-like microtubule-stabilizing activity (Mooberry et al., Cancer Res. 1999, 59, 653-60). A mechanism-based screening program revealed microtubule-stabilizing activity of the extract of C. mycofijiensis, and a bioassay-directed purification of this extract yielded laulimalide and isolaulimalide as the active components. Non-transformed A10 cells were treated with each compound and their effects on microtubules were observed. At 2-20 μM concentrations, laulimalide caused bundling of microtubules similar to that induced by Taxol. It also caused the polymerization of purified tubulin. While laulimalide was less potent than Taxol at causing microtubule bundling, at high concentrations it was more effective. In the same range of concentrations, the less-potent isolaulimalide caused an increase in the density of microtubules, but no microtubule bundles were observed.

In the MDA-MB-435 (human breast adenocarcinoma) cell line, laulimalide was found to induce cell cycle arrest at the G2/M transition. Other anti-microtubule drugs induce this same arrest and ultimately initiate apoptosis. One key apoptotic event is the activation of caspases, which are specific cysteine proteases. Cell lysates from laulimalide-treated cells were examined and found to contain protein degradation products associated with activation of the caspase cascade, indicating that a downstream effect of the stabilization of microtubules by laulimalide is the initiation of apoptosis (Mooberry et al., Cancer Res. 1999, 59, 653-60).

Laulimalide and isolaulimalide were evaluated for their ability to inhibit the growth of several transformed cell lines, in order to confirm the reports of their cytotoxicity (Mooberry et al., Cancer Res. 1999, 59, 653-60). Laulimalide was found to have a low-nanomolar IC₅₀ against both the drug-sensitive MDA-MB-435 and SK-OV-3 (ovarian carcinoma) cell lines. Isolaulimalide is less potent, with an IC₅₀ in the low micromolar range. Significantly, both laulimalide and isolaulimalide were also found to inhibit the proliferation of the P-gp overexpressing MDR cell line SKVLB-1, against which Taxol is completely ineffective, with resistance factors of 105 and 1.03, respectively.

| | Laulimalide | Isolaulimalide | Taxol |
|---|---|---|---|
| Cell Line | IC₅₀ (nM) | IC₅₀ (nM) | IC₅₀ (nM) |
| MDA-MB-435 | 5.73 ± 0.58 | 1970 ± 97 | 1.02 ± 0.25 |
| SK-OV-3 | 11.53 ± 0.53 | 2570 ± 290 | 1.71 ± 1.07 |
| SKVLB-1 | 1210 ± 490 | 2650 ± 1384 | >100000 |
| Resistance factor | 105 | 1.03 | >58480 |
| (SKVLB-1/SK-OV-3) | | | |

While the work described above identified laulimalide as a potent, microtubule-stabilizing potential cancer therapeutic, important discoveries since then have established it as a uniquely promising member of this class. While other microtubule-stabilizers, such as the epothilones and discodermolide, competitively inhibit the binding of radiolabeled Taxol to tubulin polymer, it was reported in 2002 that laulimalide does not compete with Taxol (Pryor et al., Biochemistry, 2002, 41, 9109-9115). This suggests that laulimalide binds in a completely new site on microtubules. In order to support this hypothesis, tubulin was incubated with an equimolar mixture of Taxol and laulimalide. The microtubules isolated from this experiment were found to contain nearly a 1:1:1 mixture of tubulin, Taxol, and laulimalide. This discovery identified laulimalide as the first member of a sub-class of microtubule stabilizers that appear to bind in a different site than Taxol. A distinct advantage of this new mode of binding is that laulimalide retains activity against a second class of MDR cell lines: those that are resistant to Taxol, the epothilones, and other drugs due to mutations in β-tubulin, the tubulin subunit to which these drugs bind (Pryor et al., Biochemistry, 2002, 41, 9109-9115). Computational studies have indeed suggested that laulimalide's binding site is located on α-tubulin, and is distinct from the Taxol site (Pineda et al., Bioorg. Med. Chem. Lett. 2004, 14, 4825-9).

Recent studies have revealed that laulimalide not only interferes with mitosis, induces apoptosis, and inhibits cell proliferation, but also demonstrates a cluster of effects that contribute to the inhibition of angiogenesis (Lu et al., Mol. Pharmacol 2006, 69, 1207-15). This activity is also relevant to cancer treatment, as angiogenesis, or the sprouting of capillaries from preexisting blood vessels, plays a key role in tumor growth and metastasis. Many of the drugs that interfere with microtubule dynamics, including Taxol and Taxotere, have also been found to have antiangiogenic activity (Belotti et al., Clin. Cancer Res. 1996, 2, 1843-9; Hotchkiss et al., Mol. Cancer Ther. 2002, 1, 1191-1200).

Essential to the process of angiogenesis is the activation, migration, proliferation, and differentiation of endothelial cells. In this study, the effects of laulimalide on human umbilical vein endothelial cells (HUVEC) were observed. Laulimalide inhibited HUVEC proliferation with an IC₅₀ of 4 nM. Moreover, it inhibited tubule formation and VEGF-induced HUVEC migration at subnanomolar concentrations, and did so synergistically when used in combination with Taxotere. Laulimalide also inhibited downstream signaling events related to migration and suppressed the activation of integrins, transmembrane cell adhesion proteins that mediate cell-cell and cell-matrix signaling events that are critical to migration. These results suggest that laulimalide, in addition to its other exciting properties, could induce a therapeutically relevant suppression of angiogenesis.

Given the promising pre-clinical profile and the limited supply of laulimalide, the discovery of synthetic methods to prepare novel analogs and eventually manufacture drug substances on a commercial scale would be a tremendous advance in the art.

### SUMMARY OF THE INVENTION

Presented herein are compounds and methods for their preparation, and methods for their use in the treatment of proliferative diseases and inflammatory disorders. The compounds and compositions described herein are useful as microtubule stabilizing agents and are used in the treatment of various cancers. Described herein are laulimalide derivatives and desmethyl laulimalide derivatives useful in the treatment of proliferative diseases and inflammatory disorders.

Presented herein are compounds of Formula (V) or a pharmaceutically acceptable solvate, pharmaceutically acceptable salt, or pharmaceutically acceptable prodrug thereof:

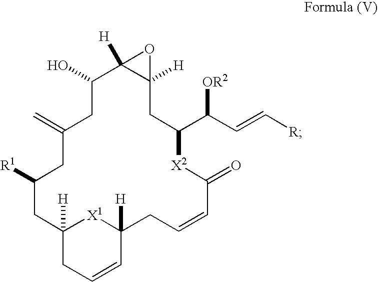

wherein:
R is selected from:

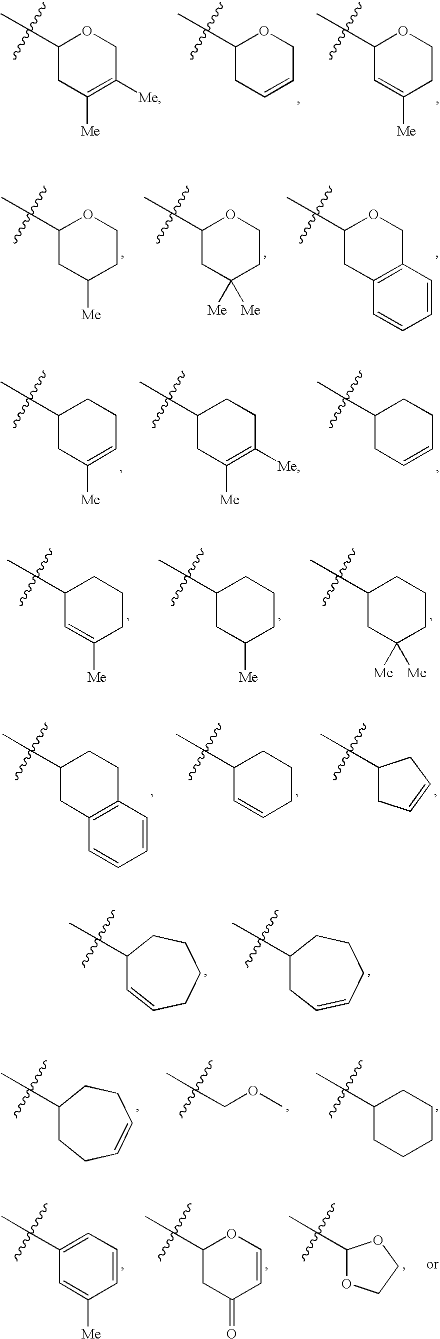

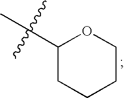

R¹ is H or methyl;
R² is H, methyl or COCH₃;
X¹ is O, NH or N-methyl; and
X² is O, NH or N-methyl.

In one aspect, is a process which provides access to laulimalide or laulimalide analogs.

In some embodiments, is a process for the preparation of a compound of Formula (I) comprising presenting a compound of Formula (II) in a reactor and subjecting the compound of Formula (II) to a cross-metathesis reaction with a cross-metathesis reactive alkene, said cross-metathesis reaction being promoted by a cross-metathesis catalyst, to produce the compound of Formula (I); wherein the compound of Formula (I) and the compound of Formula (II) are as described below:

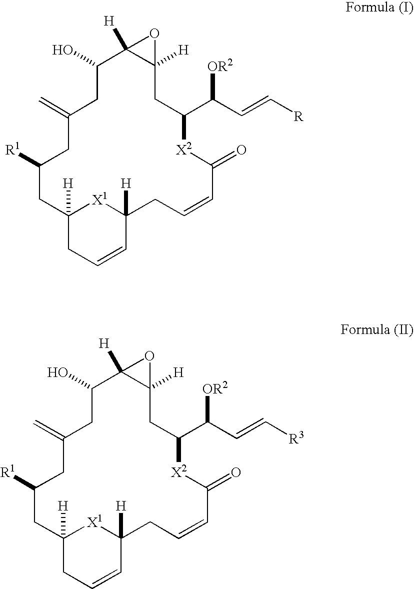

R is an optionally substituted C₁-C₁₀ alkyl, an optionally substituted C₂-C₁₀ alkenyl, an optionally substituted C₃-C₈ cycloalkyl, an optionally substituted C₄-C₈ cycloalkenyl, an optionally substituted aryl, an optionally substituted heterocycloalkyl, an optionally substituted heterocycloalkenyl, an optionally substituted heteroaryl;
   R¹ is H or methyl;
   R² is H, methyl or COCH₃;
   R³ is H, methyl, ethyl, propyl, butyl, pentyl, cyclohexyl, isopropyl or methoxymethyl;
   X¹ is O, NH or N-alkyl; and
   X² is O, NH or N-alkyl.

In other embodiments, is process for the preparation of a compound of Formula (I) comprising presenting a compound of Formula (III) in a reactor, subjecting the compound of Formula (III) to a cross-metathesis reaction with a cross-metathesis reactive alkene, said cross-metathesis reaction being promoted by a cross-metathesis catalyst, and processing the product of the cross-metathesis reaction to obtain a compound of Formula (I); wherein the compound of Formula (I) and the compound of Formula (III) are as described below:

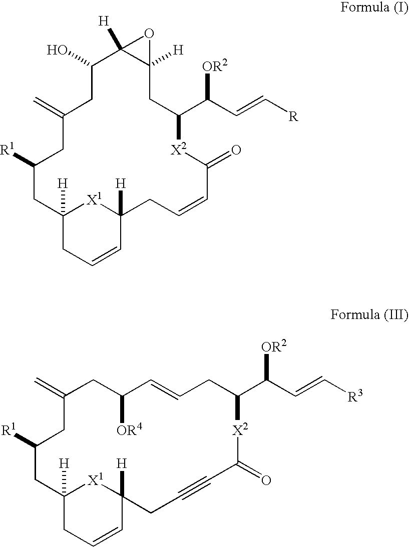

R is an optionally substituted C₁-C₁₀ alkyl, an optionally substituted C₂-C₁₀ alkenyl, an optionally substituted C₃-C₈ cycloalkyl, an optionally substituted C₄-C₈ cycloalkenyl, an optionally substituted aryl, an optionally substituted heterocycloalkyl, an optionally substituted heterocycloalkenyl, an optionally substituted heteroaryl;
   R¹ is H or methyl;
   R² is H, methyl or COCH₃;
   R³ is H, methyl, ethyl, propyl, butyl, pentyl, cyclohexyl, isopropyl or methoxymethyl;
   R⁴ is H or CH₂OCH₃;
   X¹ is O, NH or N-alkyl; and
   X² is O, NH or N-alkyl.

In an additional embodiment where R⁴ of Formula (III) is CH₂OCH₃, the process further comprises reducing an alkyne at C2-C3 to a cis-alkene by hydrogenation, removing the methoxymethyl group of R⁴ and epoxidizing an alkene at C16-C17.

In yet another embodiment where R⁴ of Formula (III) is H, the process further comprises reducing an alkyne at C2-C3 to a cis-alkene by hydrogenation and epoxidizing an alkene at C16-C17.

In a further embodiment, is process for the preparation of a compound of Formula (I) comprising presenting a compound of Formula (IV) in a reactor, subjecting the compound of Formula (IV) to a cross-metathesis reaction with a cross-metathesis reactive alkene, said cross-metathesis reaction being promoted by a cross-metathesis catalyst, and processing the product of the cross-metathesis reaction to obtain a compound of Formula (I); wherein the compound of Formula (I) and the compound of Formula (IV) are as described below:

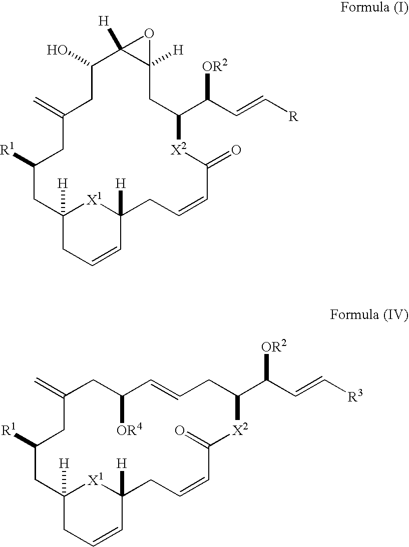

R is an optionally substituted C₁-C₁₀ alkyl, an optionally substituted C₂-C₁₀ alkenyl, an optionally substituted C₃-C₈ cycloalkyl, an optionally substituted C₄-C₈ cycloalkenyl, an optionally substituted aryl, an optionally substituted heterocycloalkyl, an optionally substituted heterocycloalkenyl, an optionally substituted heteroaryl;
   R¹ is H or methyl;
   R² is H, methyl or COCH₃;
   R³ is H, methyl, ethyl, propyl, butyl, pentyl, cyclohexyl, isopropyl or methoxymethyl;
   R⁴ is H or CH₂OCH₃;
   X¹ is O, NH or N-alkyl; and
   X² is O, NH or N-alkyl.

In an additional embodiment where R⁴ of Formula (IV) is CH₂OCH₃, the process further comprises removing the methoxymethyl group of R⁴ and epoxidizing an alkene at C16-C17.

In a further embodiment where R⁴ of Formula (IV) is H, the process further comprises epoxidizing an alkene at C16-C17.

In certain embodiments of the processes described herein the cross-metathesis reactive alkene is CH₂═CH—Y, where Y is selected from the group:

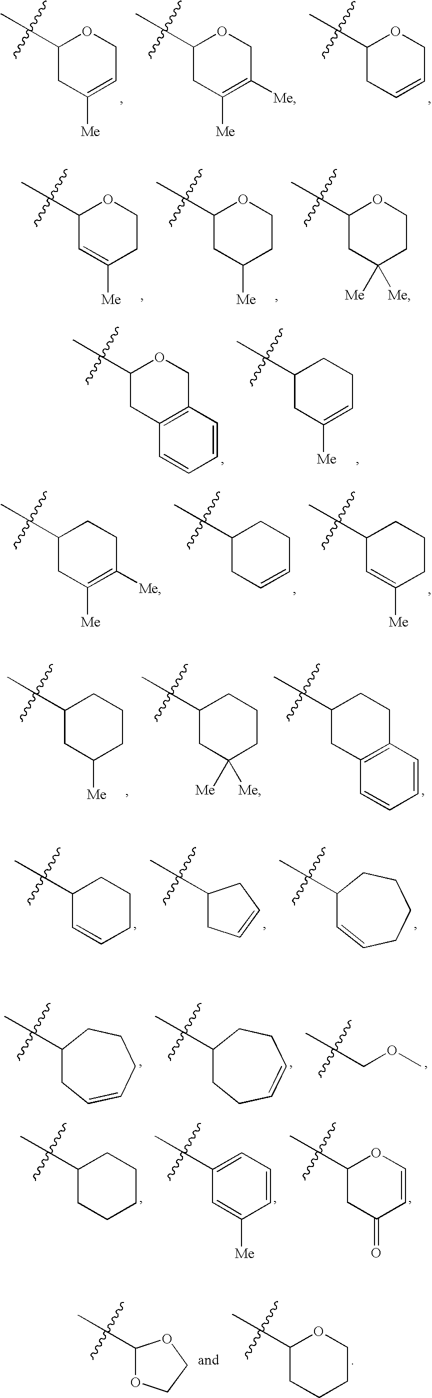

In some embodiments of the processes described herein the cross-metathesis catalyst is selected from a catalyst containing Ni, W, Ru or Mo metal. In certain embodiments of the processes described herein the cross-metathesis catalyst is dichloro(phenylmethylene)bis(tricyclohexylphosphine)ruthenium(II). In other embodiments of the processes described herein the cross-metathesis catalyst is [1,3-bis-(2,4,6-trimethylphenyl)-2-imidazolidinylidene]dichloro(phenylmethylene)(tricyclohexylphosphine)ruthenium(II). In some embodiments of the processes described herein the cross-metathesis catalyst is dichloro[[2-(1-methylethoxy)phenyl]methylene](tricyclohexylphosphine)ruthenium(II). In certain embodiments of the processes described herein the cross-metathesis catalyst is [1,3-bis(2,4,6-trimethylphenyl)-2-imidazolidinylidene]dichloro[[2-(1-methylethoxy)phenyl]methylene]ruthenium(II). In specific embodiments of the processes described herein the cross-metathesis catalyst is [1,3-bis(2-methylphenyl)-2-imidazolidinylidene]dichloro[[2-(1-methylethoxy)phenyl]methylene]ruthenium(II). In some embodiments of the processes described herein the cross-metathesis catalyst is [1,3-bis(2,4,6-trimethylphenyl)-2-imidazolidinylidene]dichloro[3-(2-pyridinyl-κN)propylidene-κC]ruthenium (II). In certain embodiments of the processes described herein the cross-metathesis catalyst is [1,3-bis(2-methylphenyl)-2-imidazolidinylidene]dichloro(benzylidene)(tricyclohexylphosphine)ruthenium (II). In some embodiments of the processes described herein the cross-metathesis catalyst is dichloro(3-methyl-2-butenylidene)bis(tricyclohexylphosphine)ruthenium(II). In other embodiments of the processes described herein the cross-metathesis catalyst is [1,3-bis(2,4,6-trimethylphenyl)-2-imidazolidinylidene]dichloro(3-methyl-2-butenylidene)(tricyclohexylphosphine)ruthenium(II). In some embodiments of the processes described herein the cross-metathesis catalyst is [1,3-bis(2,4,6-trimethylphenyl)-2-imidazolidinylidene]dichloro(benzylidene)bis(3-bromopyridine)ruthenium(II).

In additional embodiments of the processes described herein the reaction is conducted at a temperature of about 15° C. to about 35° C. In other embodiments of the processes described herein the reaction is conducted at a temperature of about 20° C. to about 150° C. with thermal heating. In some embodiments of the processes described herein the reaction is conducted at a temperature of about 20° C. to about 150° C. with microwave irradiation heating. In some embodiments of the processes described herein the amount of cross-metathesis catalyst is between about 1 mol % and about 40 mol %.

In one aspect is a method of treating a proliferative disease comprising administering a therapeutically effective amount of a compound of Formula (V) or a pharmaceutically acceptable solvate, pharmaceutically acceptable salt, or pharmaceutically acceptable prodrug thereof.

In another aspect is a method of treating a proliferative disease comprising administering a therapeutically effective amount of a compound produced by the processes described herein.

In one embodiment, the proliferative disease is cancer.

In another embodiment, the cancer is leukemia or myeloproliferative disorder.

In one aspect is a method of treating inflammatory disorders comprising administering a therapeutically effective amount of a compound of Formula (V) or a pharmaceutically acceptable solvate, pharmaceutically acceptable salt, or pharmaceutically acceptable prodrug thereof.

In another aspect is a method of treating inflammatory disorders comprising administering a therapeutically effective amount of a compound produced by the processes described herein.

In one embodiment, the inflammatory disorder is psoriasis, eczema, multiple sclerosis, and arthritis.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### DETAILED DESCRIPTION OF THE INVENTION

While embodiments of the present disclosure have been shown and described herein, such embodiments are provided by way of example only. It should be understood that various alternatives to the embodiments described herein are employed. It is intended that the claims define the scope of the present disclosure and that methods and structures within the scope of these claims and their equivalents are covered thereby.

The section headings used herein are for organizational purposes only and are not to be construed as limiting the subject matter described. All documents, or portions of documents, cited in the application including, without limitation, patents, patent applications, articles, books, manuals, and treatises are hereby incorporated by reference to the extent they are relevant.

### Certain Chemical Terminology

Unless defined otherwise, all technical and scientific terms used herein have the same meaning as is commonly understood to which the claimed subject matter belongs. In the event that there is a plurality of definitions for terms herein, those in this section prevail. Where reference is made to a URL or other such identifier or address, it is understood that such identifiers can change and particular information on the internet can come and go, but equivalent information can be found by searching the internet or other appropriate reference source. Reference thereto evidences the availability and public dissemination of such information.

It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a”, “an” and “the” include plural referents unless the context clearly dictates otherwise. It should also be noted that use of “or” means “and/of” unless stated otherwise. Furthermore, use of the term “including” as well as other forms, such as “include”, “includes”, and “included” is not limiting.

In some embodiments, definition of standard chemistry terms are found in reference works, including Carey and Sundberg “ADVANCED ORGANIC CHEMISTRY 4^{TH} ED.” Vols. A (2000) and B (2001), Plenum Press, New York. Unless otherwise indicated, conventional methods of mass spectroscopy, NMR, HPLC, IR and UV/Vis spectroscopy and pharmacology are employed. Unless specific definitions are provided, the nomenclature employed in connection with, and the laboratory procedures and techniques of, analytical chemistry, synthetic organic chemistry, and medicinal and pharmaceutical chemistry described herein are used. Standard techniques can be used for chemical syntheses, chemical analyses, pharmaceutical preparation, formulation, and delivery, and treatment of patients. In some embodiments, reactions and purification techniques are performed e.g., using kits of manufacturer's specifications or as described herein. In some embodiments, throughout the specification, groups and substituents thereof are chosen to provide stable moieties and compounds.

Where substituent groups are specified by their conventional chemical formulas, written from left to right, they equally encompass the chemically identical substituents that would result from writing the structure from right to left. As a non-limiting example, −CH₂— is equivalent to OCH₂—.

Unless otherwise noted, the use of general chemical terms, such as though not limited to “alkyl,” “amine,” “aryl,” are equivalent to their optionally substituted forms. For example, “alkyl,” as used herein, includes optionally substituted alkyl.

In some embodiments, the compounds presented herein possess one or more stereocenters and each center exist in the R or S configuration, or combinations thereof. Likewise, in other embodiments, the compounds presented herein possess one or more double bonds and each exists in the E (trans) or Z (cis) configuration, or combinations thereof. Presentation of one particular stereoisomer, regioisomer, diastereomer, enantiomer or epimer includes all possible stereoisomers, regioisomers, diastereomers, enantiomers or epimers and mixtures thereof. Thus, the compounds presented herein include all separate configurational stereoisomeric, regioisomeric, diastereomeric, enantiomeric, and epimeric forms as well as the corresponding mixtures thereof. The compounds presented herein include racemic mixtures, in all ratios, of stereoisomeric, regioisomeric, diastereomeric, enantiomeric, and epimeric forms. Techniques for inverting or leaving unchanged a particular stereocenter, and those for resolving mixtures of stereoisomers, or racemic mixtures, are contemplated herein. See, for example, Furniss et al. (eds.), VOGEL'S ENCYCLOPEDIA OF PRACTICAL ORGANIC CHEMISTRY 5^{TH} ED., Longman Scientific and Technical Ltd., Essex, 1991, 809-816; and Heller, Acc. Chem. Res. 1990, 23, 128.

In further embodiments, the compounds presented herein exist as tautomers. Tautomers are compounds that are interconvertible by migration of a hydrogen atom, accompanied by a switch of a single bond and adjacent double bond. In solutions where tautomerization is possible, a chemical equilibrium of the tautomers will exist. The exact ratio of the tautomers depends on several factors, including temperature, solvent, and pH. Some examples of tautomeric pairs include:

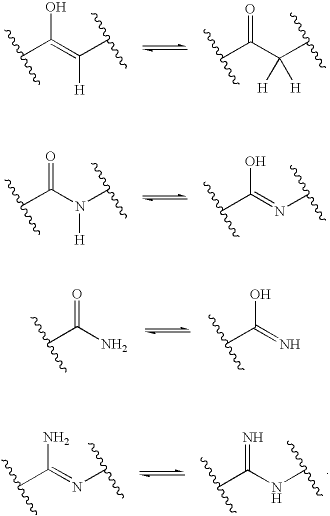

The terms “moiety”, “chemical moiety”, “group” and “chemical group”, as used herein refer to a specific segment or functional group of a molecule. Chemical moieties are often recognized chemical entities embedded in or appended to a molecule.

The term “bond” or “single bond” refers to a chemical bond between two atoms, or two moieties when the atoms joined by the bond are considered to be part of larger substructure.

As used herein, the term “optionally substituted” means that the subsequently described group may or may not bear substituents. For example, “optionally substituted alkyl” means either “alkyl” or “substituted alkyl” as defined below. Further, an optionally substituted group may be un-substituted (e.g., —CH₂CH₃), fully substituted (e.g., —CF₂CF₃), mono-substituted (e.g., —CH₂CH₂F) or substituted at a level anywhere in-between fully substituted and mono-substituted (e.g., —CH₂CHF₂, —CH₂CF₃, —CF₂CH₃, —CFHCHF₂, etc). It will be understood by those skilled in the art with respect to any group containing one or more substituents that such groups are not intended to introduce any substitution or substitution patterns (e.g., substituted alkyl includes optionally substituted cycloalkyl groups, which in turn are defined as including optionally substituted alkyl groups, potentially ad infinitum) that are sterically impractical and/or synthetically non-feasible. Thus, any substituents described should generally be understood as having a maximum molecular weight of about 500 daltons, and more typically, up to about 250 daltons. Groups that may function as substituents include, but are not limited to alkyl, hydroxy, alkoxy, amino, alkylamino, substituted amino, halogens, aryls, heteroaryl, heterocycloalkyl, acyloxy, acylamino, and sulfonylamino.

As used herein, C₁-Cₓ includes C₁-C₂, C₁-C₃ . . . C₁-Cₓ. By way of example only, a group designated as “C₁-C₄” indicates that there are one to four carbon atoms in the moiety, i.e. groups containing 1 carbon atom, 2 carbon atoms, 3 carbon atoms or 4 carbon atoms, as well as the ranges C₁-C₂ and C₁-C₃. Thus, by way of example only, “C₁-C₄ alkyl” indicates that there are one to four carbon atoms in the alkyl group, i.e., the alkyl group is selected from among methyl, ethyl, propyl, iso-propyl, n-butyl, iso-butyl, sec-butyl, and t-butyl. Whenever it appears herein, a numerical range such as “1 to 10” refers to each integer in the given range; e.g., “1 to 10 carbon atoms” means that the group may have 1 carbon atom, 2 carbon atoms, 3 carbon atoms, 4 carbon atoms, 5 carbon atoms, 6 carbon atoms, 7 carbon atoms, 8 carbon atoms, 9 carbon atoms, or 10 carbon atoms.

The term “hydrocarbon” as used herein, alone or in combination, refers to the compound or chemical group containing only carbon and hydrogen atoms.

The terms “heteroatom” or “hetero” as used herein, alone or in combination, refer to an atom other than carbon or hydrogen. In other embodiments, heteroatoms are independently selected from among oxygen, nitrogen, sulfur, phosphorous, silicon, selenium and tin but are not limited to these atoms. In some embodiments, two or more heteroatoms are present, the two or more heteroatoms are the same as each another, or some or all of the two or more heteroatoms are each different from the others.

The term “alkyl” as used herein, alone or in combination, refers to an optionally substituted straight-chain, or optionally substituted branched-chain saturated hydrocarbon monoradical having from one to about ten carbon atoms, more preferably one to six carbon atoms. Examples include, but are not limited to methyl, ethyl, n-propyl, isopropyl, 2-methyl-1-propyl, 2-methyl-2-propyl, 2-methyl-1-butyl, 3-methyl-1-butyl, 2-methyl-3-butyl, 2,2-dimethyl-1-propyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, sec-butyl, t-butyl, n-pentyl, isopentyl, neopentyl, tert-amyl and hexyl, and longer alkyl groups, such as heptyl, octyl and the like. Whenever it appears herein, a numerical range such as “C₁-C₆ alkyl” or “C₁₋₆ alkyl”, means that the alkyl group may consist of 1 carbon atom, 2 carbon atoms, 3 carbon atoms, 4 carbon atoms, 5 carbon atoms or 6 carbon atoms, although the present definition also covers the occurrence of the term “alkyl” where no numerical range is designated.

The term “alkoxy” as used herein, alone or in combination, refers to an alkyl ether radical, —O-alkyl, including the groups —O-aliphatic and —O-carbocyclyl, wherein the alkyl, aliphatic and carbocyclyl groups may be optionally substituted, and wherein the terms alkyl, aliphatic and carbocyclyl are as defined herein. Non-limiting examples of alkoxy radicals include methoxy, ethoxy, n-propoxy, isopropoxy, n-butoxy, iso-butoxy, sec-butoxy, tert-butoxy and the like.

The term “alkynyl” as used herein, alone or in combination, refers to an optionally substituted straight-chain or optionally substituted branched-chain hydrocarbon monoradical having one or more carbon-carbon triple-bonds and having from two to about ten carbon atoms, in some embodiments, from two to about six carbon atoms. Examples include, but are not limited to ethynyl, 2-propynyl, 2-butynyl, 1,3-butadiynyl and the like. In further embodiments, whenever it appears herein, a numerical range such as “C₂-C₆ alkynyl” or “C₂₋₆ alkynyl”, means that the alkynyl group consists of 2 carbon atoms, 3 carbon atoms, 4 carbon atoms, 5 carbon atoms or 6 carbon atoms, although the present definition also covers the occurrence of the term “alkynyl” where no numerical range is designated.

The term “aliphatic” as used herein, alone or in combination, refers to an optionally substituted, straight-chain or branched-chain, non-cyclic, saturated, partially unsaturated, or fully unsaturated nonaromatic hydrocarbon. Thus, the term collectively includes alkyl, alkenyl and alkynyl groups.

The terms “haloalkyl”, “haloalkenyl” and “haloalkynyl” as used herein, alone or in combination, refer to optionally substituted alkyl, alkenyl and alkynyl groups respectively, as defined above, in which one or more hydrogen atoms is replaced by fluorine, chlorine, bromine or iodine atoms, or combinations thereof. In some embodiments two or more hydrogen atoms are replaced with halogen atoms that are the same as each another (e.g. difluoromethyl); in other embodiments two or more hydrogen atoms are replaced with halogen atoms that are not all the same as each other (e.g. 1-chloro-1-fluoro-1-iodoethyl). Non-limiting examples of haloalkyl groups are fluoromethyl and bromoethyl. A non-limiting example of a haloalkenyl group is bromoethenyl. A non-limiting example of a haloalkynyl group is chloroethynyl.

The terms “cycle”, “cyclic”, “ring” and “membered ring” as used herein, alone or in combination, refer to any covalently closed structure, including alicyclic, heterocyclic, aromatic, heteroaromatic and polycyclic fused or non-fused ring systems as described herein. In some embodiments, rings are optionally substituted. In some embodiments rings form part of a fused ring system. The term “membered” is meant to denote the number of skeletal atoms that constitute the ring. Thus, by way of example only, cyclohexane, pyridine, pyran and pyrimidine are six-membered rings and cyclopentane, pyrrole, tetrahydrofuran and thiophene are five-membered rings.

The term “fused” as used herein, alone or in combination, refers to cyclic structures in which two or more rings share one or more bonds.

The term “cycloalkyl” as used herein, alone or in combination, refers to an optionally substituted, saturated, hydrocarbon monoradical ring, containing from three to about fifteen ring carbon atoms or from three to about ten ring carbon atoms, though includes additional, non-ring carbon atoms as substituents (e.g. methylcyclopropyl). In some embodiments whenever it appears herein, a numerical range such as “C₃-C₆ cycloalkyl” or “C₃₋₆ cycloalkyl”, means that the cycloalkyl group consists of 3 carbon atoms, 4 carbon atoms, 5 carbon atoms or 6 carbon atoms, i.e., is cyclopropyl, cyclobutyl, cyclopentyl or cyclohepty, although the present definition also covers the occurrence of the term “cycloalkyl” where no numerical range is designated. The term includes fused, non-fused, bridged and spiro radicals. In some embodiments, a fused cycloalkyl contains from two to four fused rings where the ring of attachment is a cycloalkyl ring, and in other embodiments, the other individual rings is alicyclic, heterocyclic, aromatic, heteroaromatic or any combination thereof. Examples include, but are not limited to cyclopropyl, cyclopentyl, cyclohexyl, decalinyl, and bicyclo[2.2.1]heptyl and adamantyl ring systems. Illustrative examples include, but are not limited to the following moieties:

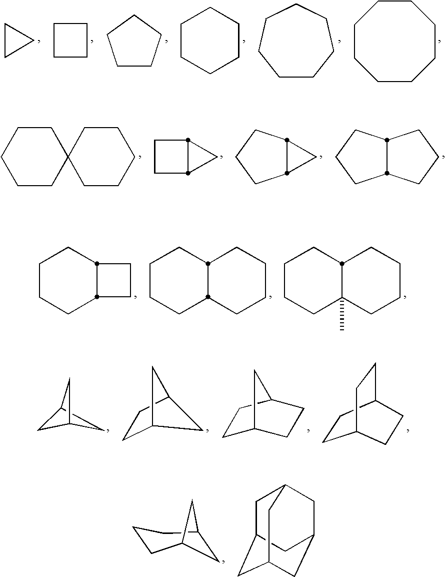

and the like.

The term “alkenyl” as used herein, alone or in combination, refers to an optionally substituted straight-chain, or optionally substituted branched-chain hydrocarbon monoradical having one or more carbon-carbon double-bonds and having from two to about ten carbon atoms, more preferably two to about six carbon atoms. The group may be in either the cis or trans conformation about the double bond(s), and should be understood to include both isomers. Examples include, but are not limited to ethenyl (—CH═CH₂), propenyl (—CH₂CH═CH₂), isopropenyl [—C(CH₃)═CH₂], butenyl, 1,3-butadienyl and the like. Whenever it appears herein, a numerical range such as “C₂-C₆ alkenyl” or “C₂₋₆ alkenyl”, means that the alkenyl group may consist of 2 carbon atoms, 3 carbon atoms, 4 carbon atoms, 5 carbon atoms or 6 carbon atoms, although the present definition also covers the occurrence of the term “alkenyl” where no numerical range is designated.

The term “cycloalkenyl” as used herein, alone or in combination, refers to an optionally substituted hydrocarbon non-aromatic, monoradical ring, having one or more carbon-carbon double-bonds and from three to about twenty ring carbon atoms, three to about twelve ring carbon atoms, or from three to about ten ring carbon atoms. The term includes fused, non-fused, bridged and spiro radicals. In some embodiments, fused cycloalkenyl contains from two to four fused rings where the ring of attachment is a cycloalkenyl ring, and in other embodiments, the other individual rings are alicyclic, heterocyclic, aromatic, heteroaromatic or any combination thereof. In some embodiments fused ring systems are fused across a bond that is a carbon-carbon single bond or a carbon-carbon double bond. Examples of cycloalkenyls include, but are not limited to cyclohexenyl, cyclopentadienyl and bicyclo[2.2.1]hept-2-ene ring systems. Illustrative examples include, but are not limited to the following moieties:

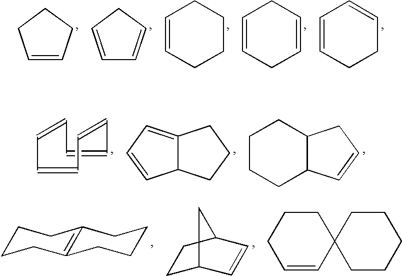

and the like.

The terms “heterocycloalkyl” as used herein, alone or in combination, refer to optionally substituted, saturated, partially unsaturated, or fully unsaturated nonaromatic ring monoradicals containing from three to about twenty ring atoms, where one or more of the ring atoms are an atom other than carbon, independently selected from among oxygen, nitrogen, sulfur, phosphorous, silicon, selenium and tin but are not limited to these atoms. In some embodiments, two or more heteroatoms are present in the ring, the two or more heteroatoms are the same as each another, or some or all of the two or more heteroatoms are each be different from the others. The terms include fused, non-fused, bridged and spiro radicals. In some embodiments fused non-aromatic heterocyclic radical contains from two to four fused rings where the attaching ring is a non-aromatic heterocycle, and in other embodiments, the other individual rings are alicyclic, heterocyclic, aromatic, heteroaromatic or any combination thereof. In some embodiments fused ring systems are fused across a single bond or a double bond, as well as across bonds that are carbon-carbon, carbon-hetero atom or hetero atom-hetero atom. The terms also include radicals having from three to about twelve skeletal ring atoms, as well as those having from three to about ten skeletal ring atoms. In some embodiments attachment of a non-aromatic heterocyclic subunit to its parent molecule is via a heteroatom or a carbon atom. Likewise, in some embodiments additional substitution is via a heteroatom or a carbon atom. As a non-limiting example, in some embodiments an imidazolidine non-aromatic heterocycle is attached to a parent molecule via either of its N atoms (imidazolidin-1-yl or imidazolidin-3-yl) or any of its carbon atoms (imidazolidin-2-yl, imidazolidin-4-yl or imidazolidin-5-yl). In certain embodiments, non-aromatic heterocycles contain one or more carbonyl or thiocarbonyl groups such as, for example, oxo- and thio-containing groups. Examples include, but are not limited to pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Illustrative examples of heterocycloalkyl groups, also referred to as non-aromatic heterocycles, include:

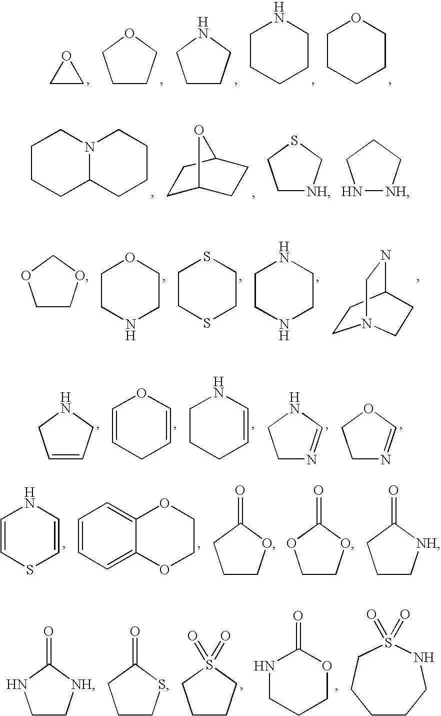

and the like.

The terms also include all ring forms of the carbohydrates, including but not limited to the monosaccharides, the disaccharides and the oligosaccharides.

The term “aromatic” as used herein, refers to a planar, cyclic or polycyclic, ring moiety having a delocalized π-electron system containing 4n+2π electrons, where n is an integer. In some embodiments aromatic rings are formed by five, six, seven, eight, nine, or more than nine atoms. In some embodiments aromatics are optionally substituted and in other embodiments are monocyclic or fused-ring polycyclic. The term aromatic encompasses both all carbon containing rings (e.g., phenyl) and those rings containing one or more heteroatoms (e.g., pyridine).

In one embodiment, the term “aryl” means, unless otherwise stated, a polyunsaturated, aromatic, hydrocarbon substituent which is a single ring or multiple rings (in some embodiments, from 1 to 3 rings) which are fused together or linked covalently. The term “heteroaryl” refers to aryl groups (or rings) that contain from one to four heteroatoms selected from N, O, and S, wherein nitrogen and sulfur atoms are optionally oxidized, and the nitrogen atom(s) are optionally quaternized. In another embodiment, the heteroaryl group is attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridinyl, 3-pyridinyl, 4-pyridinyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of above noted aryl and heteroaryl ring systems are selected from the group of acceptable substituents described below. The terms “arylene” and “heteroarylene” refer to the divalent derivatives of aryl and heteroaryl, respectively.

For brevity, the term “aryl” when used in combination with other terms (e.g., aryloxo, arylthioxo, arylalkyl) includes both aryl and heteroaryl rings as defined above. Thus, the term “arylalkyl” includes those radicals in which an aryl group is attached to an alkyl group (e.g., benzyl, phenethyl, pyridinylmethyl and the like) including those alkyl groups in which a carbon atom (e.g., a methylene group) has been replaced by, for example, an oxygen atom (e.g., phenoxymethyl, 2-pyridinyloxymethyl, 3-(1-naphthyloxy)propyl, and the like). Similarly, the term “heteroarylalkyl” includes those radicals in which a heteroaryl group is attached to an alkyl group (e.g., pyridinylmethyl, quinolinylmethyl, 1,2,4-triazolyl[4,3-b]pyridazinylmethyl, 1H-benzotriazolylmethyl, benzothiazolylmethyl, and the like. However, the term “haloaryl,” as used herein covers only aryls substituted with one or more halogens.

The term “heteroaryl” as used herein, alone or in combination, refers to optionally substituted aromatic monoradicals containing from about five to about twenty skeletal ring atoms, where one or more of the ring atoms is a heteroatom independently selected from among oxygen, nitrogen, sulfur, phosphorous, silicon, selenium and tin but not limited to these atoms and with the proviso that the ring of said group does not contain two adjacent O or S atoms. In some embodiments two or more heteroatoms are present in the ring, in other embodiments, the two or more heteroatoms are the same as each another, or some or all of the two or more heteroatoms are each different from the others. The term heteroaryl includes optionally substituted fused and non-fused heteroaryl radicals having at least one heteroatom. The term heteroaryl also includes fused and non-fused heteroaryls having from five to about twelve skeletal ring atoms, as well as those having from five to about ten skeletal ring atoms. In some embodiments, bonding to a heteroaryl group is via a carbon atom or a heteroatom. Thus, as a non-limiting example, in some embodiments, an imidiazole group is attached to a parent molecule via any of its carbon atoms (imidazol-2-yl, imidazol-4-yl or imidazol-5-yl), or its nitrogen atoms (imidazol-1-yl or imidazol-3-yl). Likewise, in some embodiments, a heteroaryl group is further substituted via any or all of its carbon atoms, and/or any or all of its heteroatoms. In other embodiments, fused heteroaryl radical contains from two to four fused rings where the ring of attachment is a heteroaromatic ring and in other embodiments, the other individual rings are alicyclic, heterocyclic, aromatic, heteroaromatic or any combination thereof. A non-limiting example of a single ring heteroaryl group includes pyridyl; fused ring heteroaryl groups include benzimidazolyl, quinolinyl, acridinyl; and a non-fused bi-heteroaryl group includes bipyridinyl. Further examples of heteroaryls include, without limitation, furanyl, thienyl, oxazolyl, acridinyl, phenazinyl, benzimidazolyl, benzofuranyl, benzoxazolyl, benzothiazolyl, benzothiadiazolyl, benzothiophenyl, benzoxadiazolyl, benzotriazolyl, imidazolyl, indolyl, isoxazolyl, isoquinolinyl, indolizinyl, isothiazolyl, isoindolyloxadiazolyl, indazolyl, pyridyl, pyridazyl, pyrimidyl, pyrazinyl, pyrrolyl, pyrazinyl, pyrazolyl, purinyl, phthalazinyl, pteridinyl, quinolinyl, quinazolinyl, quinoxalinyl, triazolyl, tetrazolyl, thiazolyl, triazinyl, thiadiazolyl and the like, and their oxides, such as for example pyridyl-N-oxide. Illustrative examples of heteroaryl groups include the following moieties:

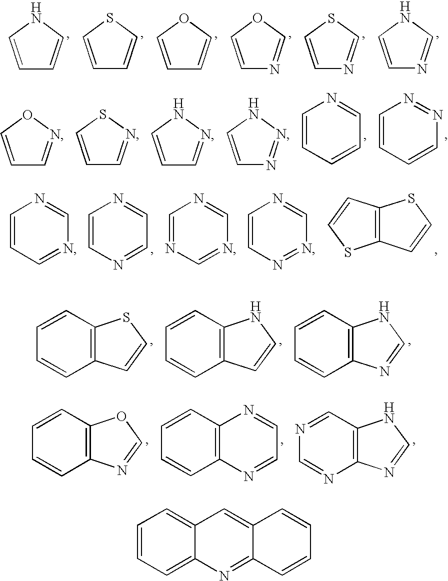

and the like.

The terms “halogen”, “halo” or “halide” as used herein, alone or in combination refer to fluoro, chloro, bromo and iodo.

The term “hydroxy” as used herein, alone or in combination, refers to the monoradical —OH.

The term “cyano” as used herein, alone or in combination, refers to the monoradical —CN.

The term “nitro” as used herein, alone or in combination, refers to the monoradical —NO₂.

The term “oxy” as used herein, alone or in combination, refers to the diradical —O—.

The term “oxo” as used herein, alone or in combination, refers to the diradical ═O.

The term “carbonyl” as used herein, alone or in combination, refers to the diradical —C(═O)—, which in other embodiments, is also written as —C(O)—.

The terms “carboxy” or “carboxyl” as used herein, alone or in combination, refer to the moiety —C(O)OH, which in some embodiments, is also written as —COOH.

The term “sulfinyl” as used herein, alone or in combination, refers to the diradical —S(═O)—.

The term “sulfonyl” as used herein, alone or in combination, refers to the diradical —S(═O)₂—.

The terms “sulfonamide”, “sulfonamido” and “sulfonamidyl” as used herein, alone or in combination, refer to the diradical groups —S(═O)₂—NH— and NH—S(═O)₂—.

The terms “sulfamide”, “sulfamido” and “sulfamidyl” as used herein, alone or in combination, refer to the diradical group —NH—S(═O)₂—NH—.

The term “reactant,” as used herein, refers to a nucleophile or electrophile used to create covalent linkages.

### Certain Pharmaceutical Terminology

The term “subject”, “patient” or “individual” as used herein in reference to individuals suffering from a disorder, and the like, encompasses mammals and non-mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish and the like. In one embodiment of the methods and compositions provided herein, the mammal is a human.

The terms “treat,” “treating” or “treatment,” and other grammatical equivalents as used herein, include alleviating, abating or ameliorating a disease or condition symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition, and are intended to include prophylaxis. The terms further include achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder. In some embodiments, for prophylactic benefit, the compositions are administered to a patient at risk of developing a particular disease, or to a patient reporting one or more of the physiological symptoms of a disease, even though a diagnosis of this disease may not have been made.

As used herein, the terms “cancer treatment” “cancer therapy” and the like encompasses treatments such as surgery, radiation therapy, administration of chemotherapeutic agents and combinations of any two or all of these methods. In some embodiments, combination treatments occur sequentially or concurrently. Treatment(s), such as radiation therapy and/or chemotherapy, that is administered prior to surgery, is referred to as neoadjuvant therapy. Treatments(s), such as radiation therapy and/or chemotherapy, administered after surgery is referred to herein as adjuvant therapy.

In some embodiments, examples of surgeries that are used for cancer treatment include, but are not limited to radical prostatectomy, cryotherapy, mastectomy, lumpectomy, transurethral resection of the prostate, and the like.

In some embodiments, the compounds described herein are administered in combination with surgery, as an adjuvant, or as a neoadjuvant agent. In other embodiments, the compounds described herein are useful in instances where radiation and chemotherapy are indicated, to enhance the therapeutic benefit of these treatments, including induction chemotherapy, primary (neoadjuvant) chemotherapy, and both adjuvant radiation therapy and adjuvant chemotherapy. Radiation and chemotherapy frequently are indicated as adjuvants to surgery in the treatment of cancer. For example, in some embodiments, radiation is used both pre- and post-surgery as components of the treatment strategy for rectal carcinoma. In further embodiments, the compounds described herein are useful following surgery in the treatment of cancer in combination with radio- and/or chemotherapy.

Where combination treatments are contemplated, it is not intended that the compounds described herein be limited by the particular nature of the combination. For example, in some embodiments, the compounds described herein are administered in combination as simple mixtures as well as chemical hybrids. An example of the latter is where the compound is covalently linked to a targeting carrier or to an active pharmaceutical. In some embodiments, covalent binding is accomplished in many ways, such as, though not limited to, the use of a commercially available cross-linking compound.

As used herein, the terms “pharmaceutical combination”, “administering an additional therapy”, “administering an additional therapeutic agent” and the like refer to a pharmaceutical therapy resulting from the mixing or combining of more than one active ingredient and includes both fixed and non-fixed combinations of the active ingredients. The term “fixed combination” means that at least one of the compounds described herein, and at least one co-agent, are both administered to a patient simultaneously in the form of a single entity or dosage. The term “non-fixed combination” means that at least one of the compounds described herein, and at least one co-agent, are administered to a patient as separate entities either simultaneously, concurrently or sequentially with variable intervening time limits, wherein such administration provides effective levels of the two or more compounds in the body of the patient. These also apply to cocktail therapies, e.g. the administration of three or more active ingredients.

As used herein, the terms “co-administration”, “administered in combination with” and their grammatical equivalents or the like are meant to encompass administration of the selected therapeutic agents to a single patient, and are intended to include treatment regimens in which the agents are administered by the same or different route of administration or at the same or different times. In some embodiments the compounds described herein is co-administered with other agents. These terms encompass administration of two or more agents to an animal so that both agents and/or their metabolites are present in the animal at the same time. They include simultaneous administration in separate compositions, administration at different times in separate compositions, and/or administration in a composition in which both agents are present. Thus, in some embodiments, the compounds described herein and the other agent(s) are administered in a single composition. In some embodiments, the compounds described herein and the other agent(s) are admixed in the composition.

The terms “effective amount”, “therapeutically effective amount” or “pharmaceutically effective amount” as used herein, refer to a sufficient amount of at least one agent or compound being administered which will relieve to some extent one or more of the symptoms of the disease or condition being treated. In some embodiments, the result is a reduction and/or alleviation of the signs, symptoms, or causes of a disease, or any other desired alteration of a biological system. For example, an “effective amount” for therapeutic uses is the amount of the composition comprising the compound as disclosed herein required to provide a clinically significant decrease in a disease. In some embodiments, an appropriate “effective” amount in any individual case is determined using techniques, such as a dose escalation study.

In some embodiments, the terms “administer,” “administering”, “administration,” and the like, as used herein, refer to the methods that are used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. In some embodiments, the administration techniques are employed with the compounds and methods described herein, e.g., as discussed in Goodman and Gilman, The Pharmacological Basis of Therapeutics, current ed.; Pergamon; and Remington's, Pharmaceutical Sciences (current edition), Mack Publishing Co., Easton, Pa. In other embodiments, the compounds and compositions described herein are administered orally.

The term “acceptable” as used herein, with respect to a formulation, composition or ingredient, means having no persistent detrimental effect on the general health of the subject being treated.

The term “pharmaceutically acceptable” as used herein, refers to a material, such as a carrier or diluent, which does not abrogate the biological activity or properties of the compounds described herein, and is relatively nontoxic, i.e., in other embodiments, the material is administered to an individual without causing undesirable biological effects or interacting in a deleterious manner with any of the components of the composition in which it is contained.

The term “pharmaceutical composition,” as used herein, refers to a biologically active compound, optionally mixed with at least one pharmaceutically acceptable chemical component, such as, though not limited to carriers, stabilizers, diluents, dispersing agents, suspending agents, thickening agents, and/or excipients.

The term “carrier” as used herein, refers to relatively nontoxic chemical compounds or agents that facilitate the incorporation of the compound into cells or tissues.

The term “agonist,” as used herein, refers to a molecule such as the compound, a drug, an enzyme activator or a hormone modulator which enhances the activity of another molecule or the activity of a receptor site.

The term “antagonist,” as used herein, refers to a molecule such as the compound, a drug, an enzyme inhibitor, or a hormone modulator, which diminishes, or prevents the action of another molecule or the activity of a receptor site.

The term “modulate,” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator,” as used herein, refers to a molecule that interacts with a target either directly or indirectly. The interactions include, but are not limited to, the interactions of an agonist and an antagonist.

The term “pharmaceutically acceptable derivative or prodrug” as used herein, refers to any pharmaceutically acceptable salt, ester, salt of an ester or other derivative of the compound of Formula (V), which, upon administration to a recipient, is capable of providing, either directly or indirectly, the compound disclosed herein or a pharmaceutically active metabolite or residue thereof. Particularly favored derivatives or prodrugs are those that increase the bioavailability of the compounds described herein when such compounds are administered to a patient (e.g., by allowing orally administered compound to be more readily absorbed into blood) or which enhance delivery of the parent compound to a biological compartment (e.g., the brain or lymphatic system).

The term “pharmaceutically acceptable salt” as used herein, refers to salts that retain the biological effectiveness of the free acids and bases of the specified compound and that are not biologically or otherwise undesirable. In some embodiments, compounds described herein possess acidic or basic groups and therefore may react with any of a number of inorganic or organic bases, and inorganic and organic acids, to form a pharmaceutically acceptable salt. In some embodiments, these salts are prepared in situ during the final isolation and purification of the compounds described herein, or by separately reacting a purified compound in its free base form with a suitable organic or inorganic acid, and isolating the salt thus formed. Examples of pharmaceutically acceptable salts include those salts prepared by reaction of the compounds described herein with a mineral or organic acid or an inorganic base, such salts including, acetate, acrylate, adipate, alginate, aspartate, benzoate, benzenesulfonate, bisulfate, bisulfite, bromide, butyrate, butyn-1,4-dioate, camphorate, camphorsulfonate, caproate, caprylate, chlorobenzoate, chloride, citrate, cyclopentanepropionate, decanoate, digluconate, dihydrogenphosphate, dinitrobenzoate, dodecylsulfate, ethanesulfonate, formate, fumarate, glucoheptanoate, glycerophosphate, glycolate, hemisulfate, heptanoate, hexanoate, hexyne-1,6-dioate, hydroxybenzoate, γ-hydroxybutyrate, hydrochloride, hydrobromide, hydroiodide, 2-hydroxyethanesulfonate, iodide, isobutyrate, lactate, maleate, malonate, methanesulfonate, mandelate. metaphosphate, methanesulfonate, methoxybenzoate, methylbenzoate, monohydrogenphosphate, 1-napthalenesulfonate, 2-napthalenesulfonate, nicotinate, nitrate, palmoate, pectinate, persulfate, 3-phenylpropionate, phosphate, picrate, pivalate, propionate, pyrosulfate, pyrophosphate, propiolate, phthalate, phenylacetate, phenylbutyrate, propanesulfonate, salicylate, succinate, sulfate, sulfite, succinate, suberate, sebacate, sulfonate, tartrate, thiocyanate, tosylate undeconate and xylenesulfonate. In some embodiments, other acids, such as oxalic, while not in themselves pharmaceutically acceptable, are employed in the preparation of salts useful as intermediates in obtaining the compounds described herein and their pharmaceutically acceptable acid addition salts. Further, in some embodiments, those compounds described herein which comprise a free acid group reacts with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Illustrative examples of bases include sodium hydroxide, potassium hydroxide, choline hydroxide, sodium carbonate, N⁺(C₁₋₄ alkyl)₄, and the like. Representative organic amines useful for the formation of base addition salts include ethylamine, diethylamine, ethylenediamine, ethanolamine, diethanolamine, piperazine and the like. It should be understood that in some embodiments, the compounds described herein also include the quaternization of any basic nitrogen-containing groups they contain. In some embodiments, water or oil-soluble or dispersible products are obtained by such quaternization.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The term “metabolite,” as used herein, refers to a derivative of the compound which is formed when the compound is metabolized.

The term “active metabolite,” as used herein, refers to a biologically active derivative of the compound that is formed when the compound is metabolized.

The term “metabolized,” as used herein, refers to the sum of the processes (including, but not limited to, hydrolysis reactions and reactions catalyzed by enzymes) by which a particular substance is changed by an organism. Thus, in some embodiments, enzymes produce specific structural alterations to the compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulphydryl groups. Further information on metabolism may be obtained from The Pharmacological Basis of Therapeutics, 9th Edition, McGraw-Hill (1996).

### Compounds

The following embodiments are provided by way of example only. Various alternatives and equivalents of these embodiments may be employed in practicing and using the described methods, compositions and processes.

Isolation of laulimalide from natural sources is not a viable production route. Synthetically, the synthesis is quite complex. More accessible analogs with similar or equivalent bioactivity will provide more utility relative to current isolation or synthesis methods. Further, the sensitivity of the C16-C17 epoxy functionality and the nucleophilicity of the parent C20 hydroxy functionality reveal the potential for analog development to yield compounds with superior physicochemical behavior and potentially superior biological activity.

In one aspect is a compound of Formula (V) or pharmaceutically acceptable solvate, pharmaceutically acceptable salt, or pharmaceutically acceptable prodrug thereof:

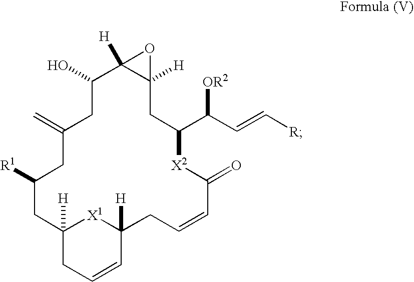

wherein:
R is selected from:

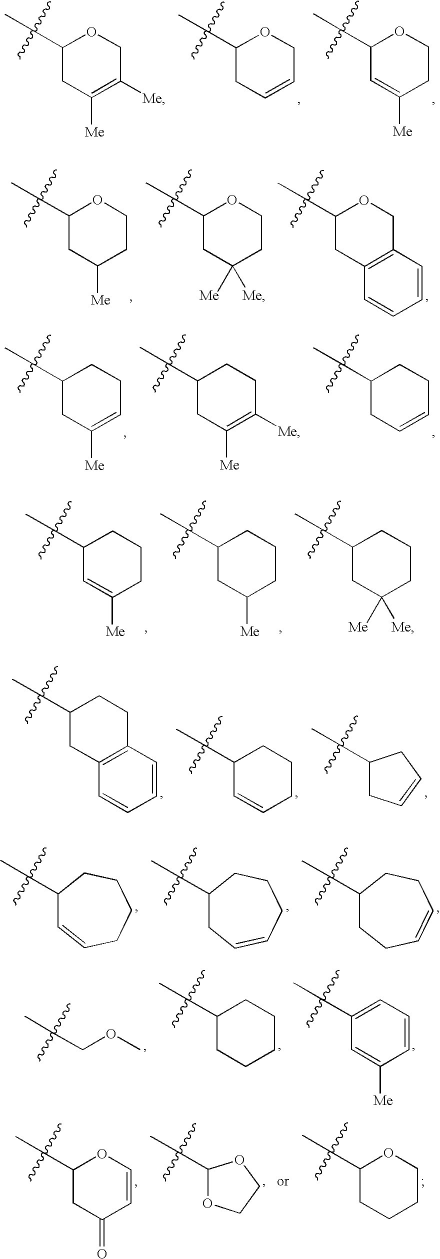

R¹ is H or methyl;
R² is H, methyl or COCH₃;
X¹ is O, NH or N-methyl; and
X² is O, NH or N-methyl.

In one embodiment R¹ is methyl. In another embodiment R¹ is H. In a further embodiment X¹ is O and X² is O. In yet a further embodiment R² is H.

In one embodiment, the compound presented herein is selected from:

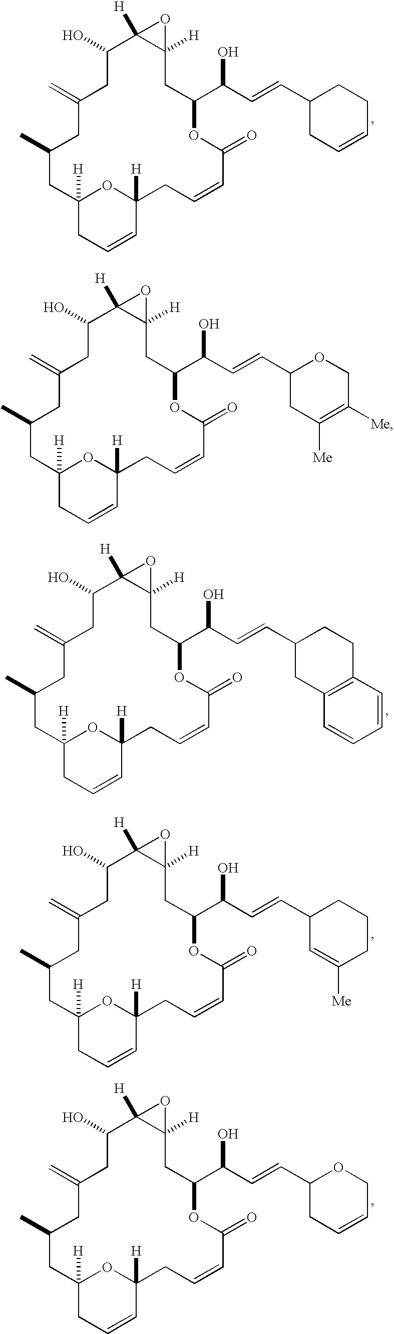

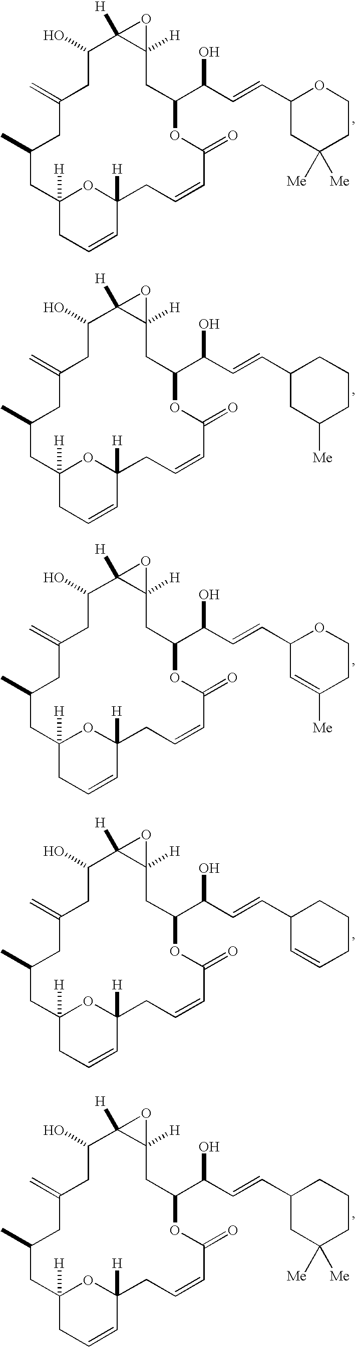

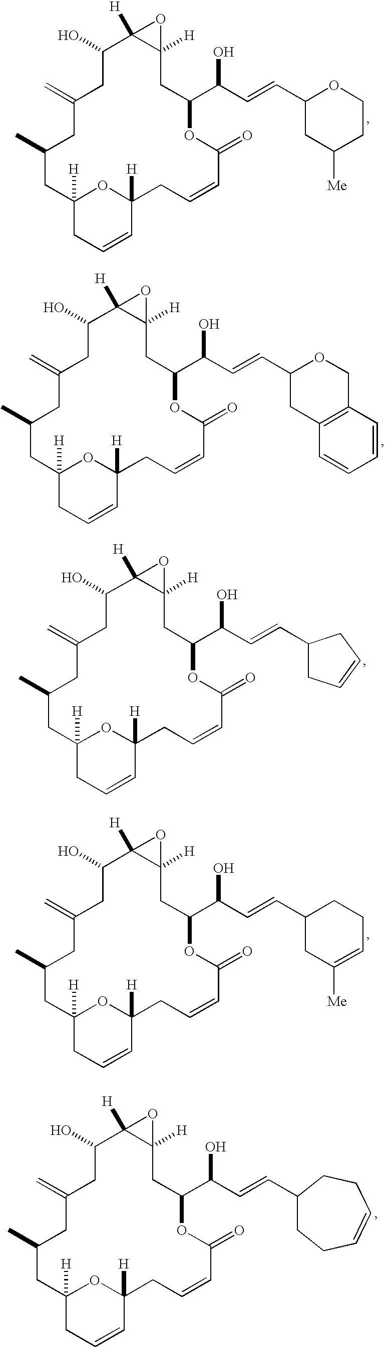

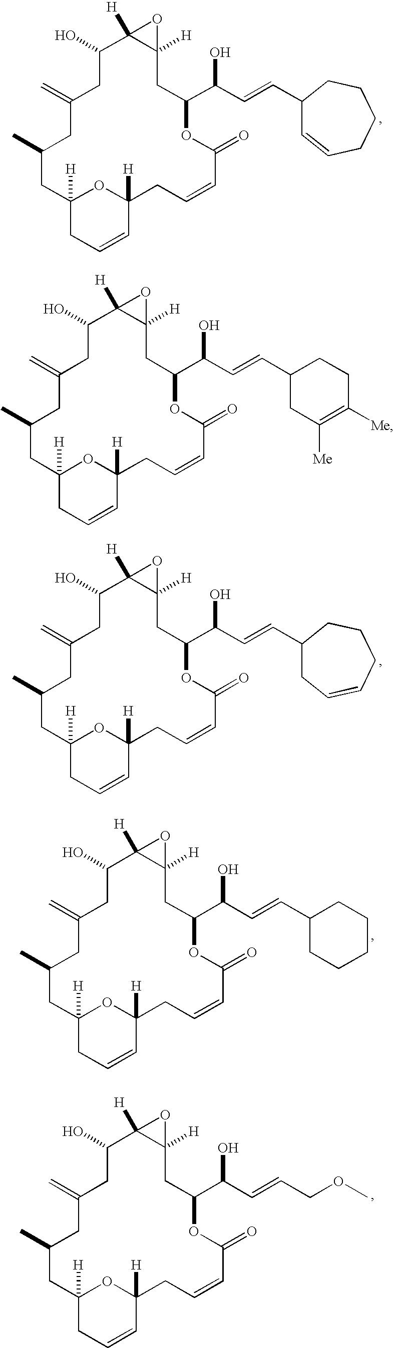

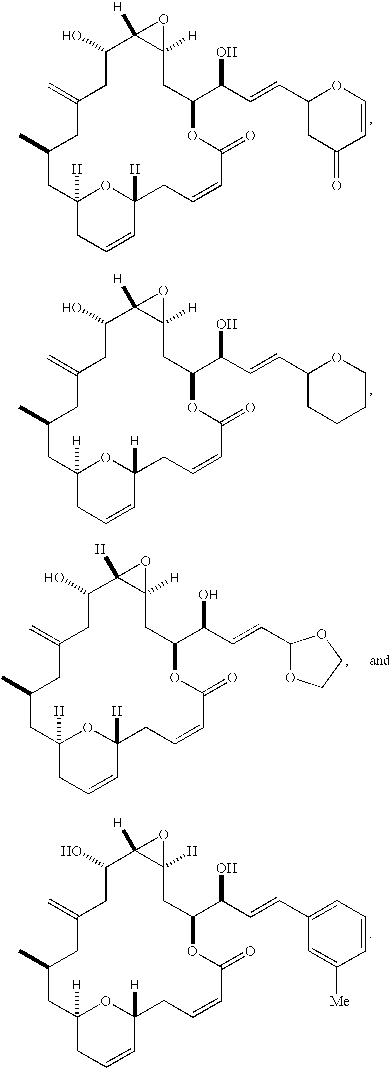

In another embodiment the compound described herein is selected from:

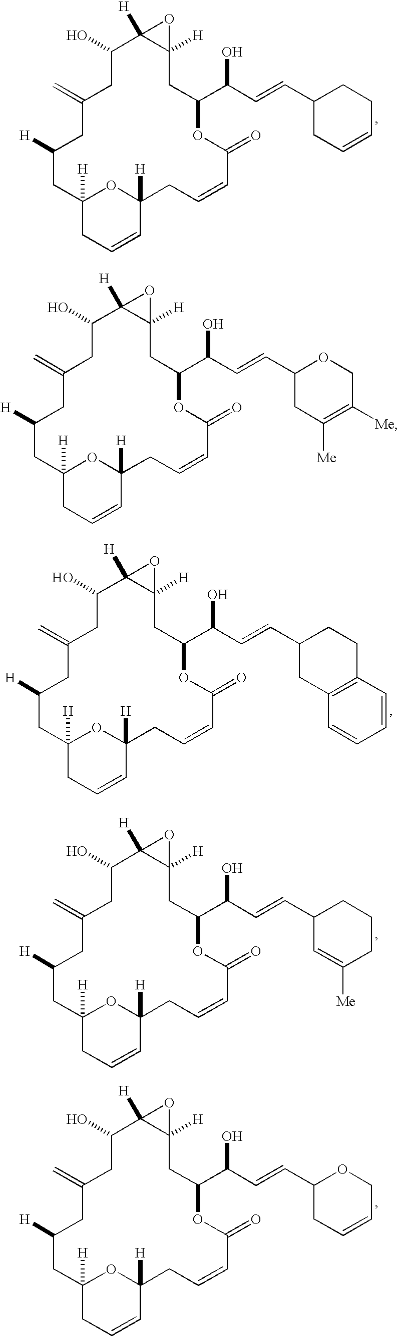

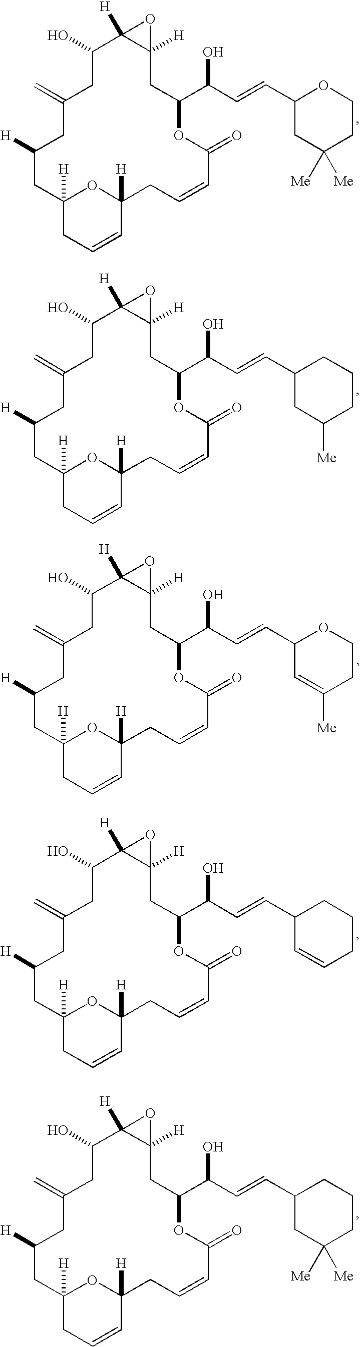

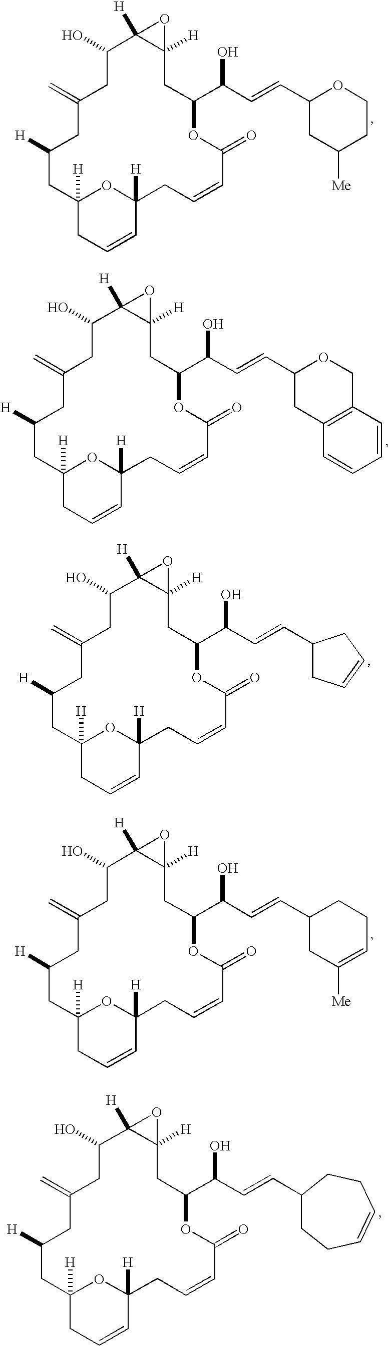

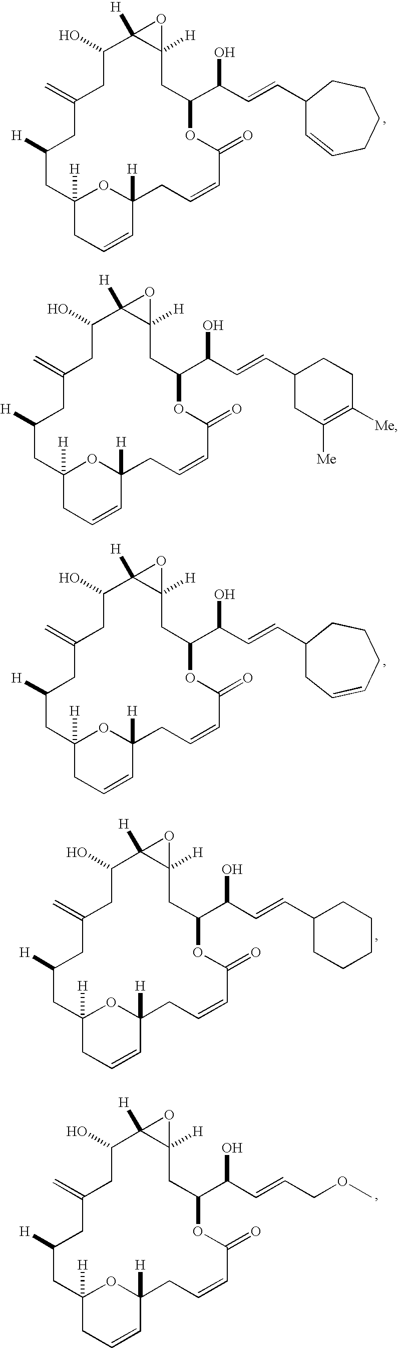

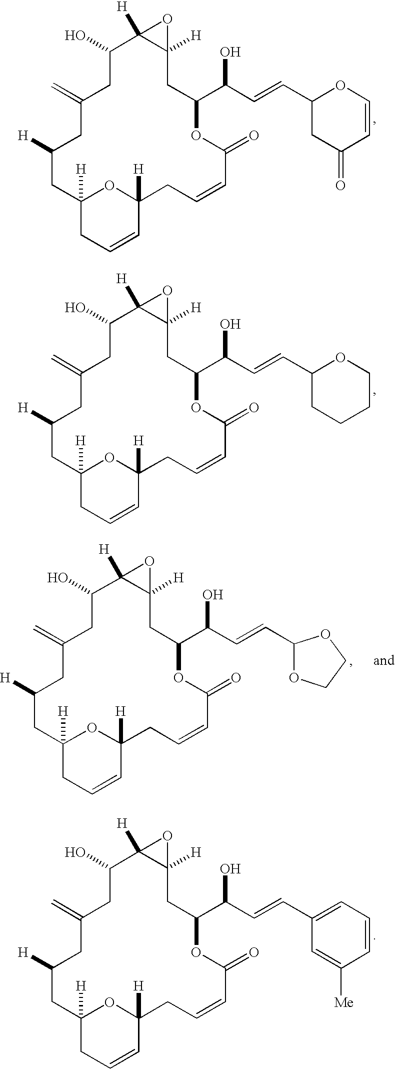

In one embodiment is a pharmaceutical composition comprising a compound of Formula (V) or a pharmaceutically acceptable solvate, pharmaceutically acceptable salt, or pharmaceutically acceptable prodrug; and a pharmaceutically acceptable carrier or excipient.

### Processes

In one aspect, is a process which provides access to laulimalide or laulimalide analogs.

In some embodiments, is a process for the preparation of a compound of Formula (I) comprising presenting a compound of Formula (II) in a reactor and subjecting the compound of Formula (II) to a cross-metathesis reaction with a cross-metathesis reactive alkene, said cross-metathesis reaction being promoted by a cross-metathesis catalyst, to produce the compound of Formula (I); wherein the compound of Formula (I) and the compound of Formula (II) are as described below:

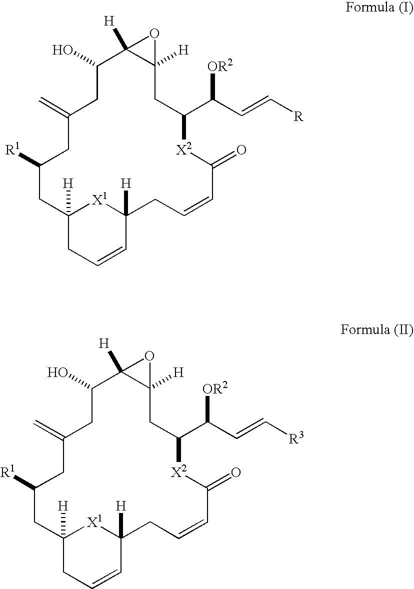

R is an optionally substituted C₁-C₁₀ alkyl, an optionally substituted C₂-C₁₀ alkenyl, an optionally substituted C₃-C₈ cycloalkyl, an optionally substituted C₄-C₈ cycloalkenyl, an optionally substituted aryl, an optionally substituted heterocycloalkyl, an optionally substituted heterocycloalkenyl, an optionally substituted heteroaryl;
   R¹ is H or methyl;
   R² is H, methyl or COCH₃;
   R³ is H, methyl, ethyl, propyl, butyl, pentyl, cyclohexyl, isopropyl or methoxymethyl;
   X¹ is O, NH or N-alkyl; and
   X² is O, NH or N-alkyl.

In other embodiments is a process for the preparation of a compound of Formula (I) comprising presenting a compound of Formula (III) in a reactor, subjecting the compound of Formula (III) to a cross-metathesis reaction with a cross-metathesis reactive alkene, said cross-metathesis reaction being promoted by a cross-metathesis catalyst, and processing the product of the cross-metathesis reaction to obtain a compound of Formula (I); wherein the compound of Formula (I) and the compound of Formula (III) are as described below:

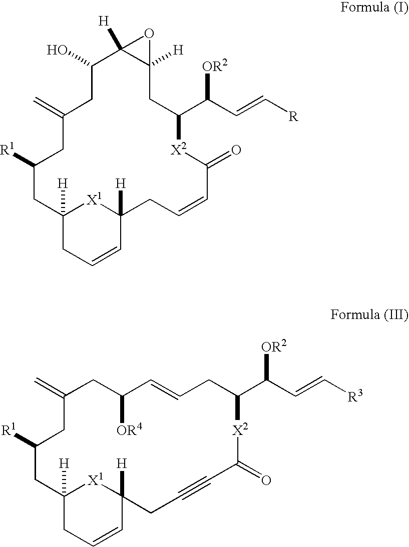

R is an optionally substituted C₁-C₁₀ alkyl, an optionally substituted C₂-C₁₀ alkenyl, an optionally substituted C₃-C₈ cycloalkyl, an optionally substituted C₄-C₈ cycloalkenyl, an optionally substituted aryl, an optionally substituted heterocycloalkyl, an optionally substituted heterocycloalkenyl, an optionally substituted heteroaryl;
   R¹ is H or methyl;
   R² is H, methyl or COCH₃;
   R³ is H, methyl, ethyl, propyl, butyl, pentyl, cyclohexyl, isopropyl or methoxymethyl;
   R⁴ is H or CH₂OCH₃;
   X¹ is O, NH or N-alkyl; and
   X² is O, NH or N-alkyl.

In an additional embodiment where R⁴ of Formula (III) is CH₂OCH₃, the process further comprises reducing an alkyne at C2-C3 to a cis-alkene by hydrogenation, removing the methoxymethyl group of R⁴ and epoxidizing an alkene at C16-C17.

In yet another embodiment where R⁴ of Formula (III) is H, the process further comprises reducing an alkyne at C2-C3 to a cis-alkene by hydrogenation and epoxidizing an alkene at C16-C17.

In a further embodiment, is a process for the preparation of a compound of Formula (I) comprising presenting a compound of Formula (IV) in a reactor, subjecting the compound of Formula (IV) to a cross-metathesis reaction with a cross-metathesis reactive alkene, said cross-metathesis reaction being promoted by a cross-metathesis catalyst, and processing the product of the cross-metathesis reaction to obtain a compound of Formula (I); wherein the compound of Formula (I) and the compound of Formula (IV) are as described below:

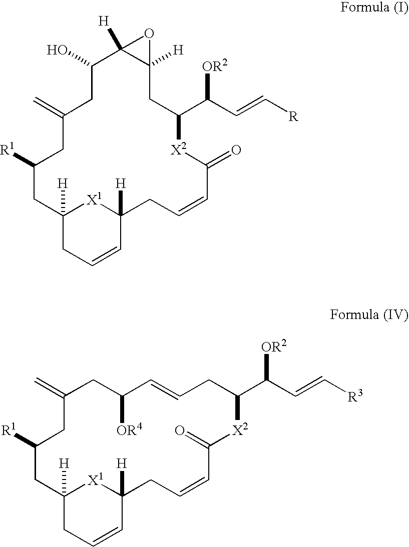

R is an optionally substituted C₁-C₁₀ alkyl, an optionally substituted C₂-C₀ alkenyl, an optionally substituted C₃-C₈ cycloalkyl, an optionally substituted C₄-C₈ cycloalkenyl, an optionally substituted aryl, an optionally substituted heterocycloalkyl, an optionally substituted heterocycloalkenyl, an optionally substituted heteroaryl;
   R¹ is H or methyl;
   R² is H, methyl or COCH₃;
   R³ is H, methyl, ethyl, propyl, butyl, pentyl, cyclohexyl, isopropyl or methoxymethyl;
   R⁴ is H or CH₂OCH₃;
   X¹ is O, NH or N-alkyl; and
   X² is O, NH or N-alkyl.

In an additional embodiment where R⁴ of Formula (IV) is CH₂OCH₃, the process further comprises removing the methoxymethyl group of R⁴ and epoxidizing an alkene at C16-C17.

In an alternative embodiment where R⁴ of Formula (IV) is H, the process further comprises epoxidizing an alkene at C16-C17.

In certain embodiments of the processes described herein the cross-metathesis reactive alkene is CH₂═CH—Y, where Y is selected from the group:

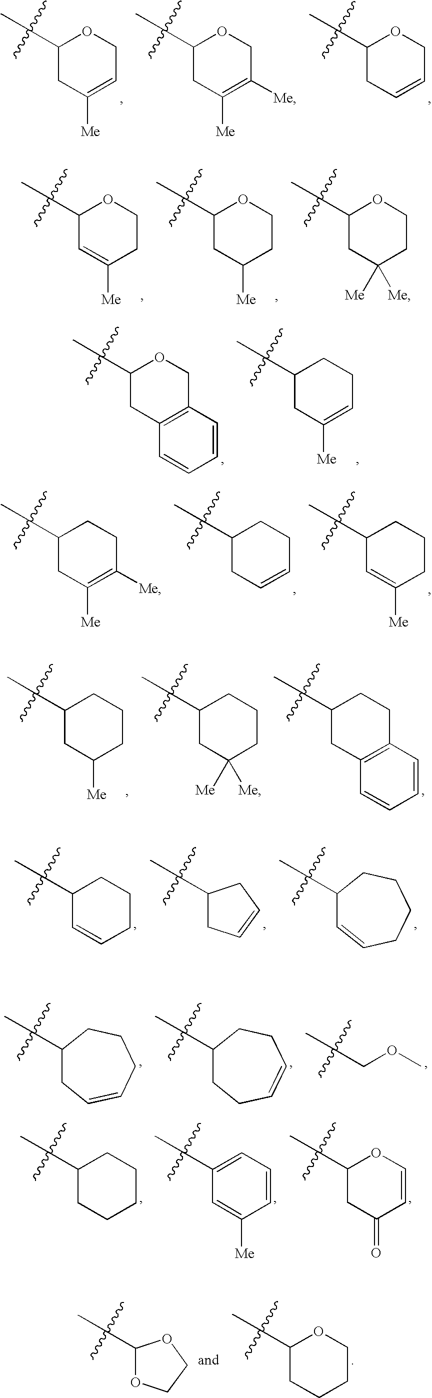

In some embodiments of the processes described herein the cross-metathesis catalyst is selected from a catalyst containing Ni, W, Ru or Mo metal. In other embodiments, the cross-metathesis catalyst is a catalyst containing Ru. In certain embodiments of the processes described herein the cross-metathesis catalyst is dichloro(phenylmethylene)bis(tricyclohexylphosphine)ruthenium(II). In specific embodiments of the processes described herein the cross-metathesis catalyst is [1,3-bis-(2,4,6-trimethylphenyl)-2-imidazolidinylidene]dichloro(phenylmethylene)(tricyclohexylphosphine)ruthenium(II). In some embodiments of the processes described herein the cross-metathesis catalyst is dichloro[[2-(1-methylethoxy)phenyl]methylene](tricyclohexylphosphine)ruthenium(II). In certain embodiments of the processes described herein the cross-metathesis catalyst is [1,3-bis(2,4,6-trimethylphenyl)-2-imidazolidinylidene]dichloro[[2-(1-methylethoxy)phenyl]methylene]ruthenium(II). In specific embodiments of the processes described herein the cross-metathesis catalyst is [1,3-bis(2-methylphenyl)-2-imidazolidinylidene]dichloro[[2-(1-methylethoxy)phenyl]methylene]ruthenium(II). In some embodiments of the processes described herein the cross-metathesis catalyst is [1,3-bis(2,4,6-trimethylphenyl)-2-imidazolidinylidene]dichloro[3-(2-pyridinyl-κN)propylidene-κC]ruthenium (II). In certain embodiments of the processes described herein the cross-metathesis catalyst is [1,3-bis(2-methylphenyl)-2-imidazolidinylidene]dichloro(benzylidene)(tricyclohexylphosphine)ruthenium (II). In some embodiments of the processes described herein the cross-metathesis catalyst is dichloro(3-methyl-2-butenylidene)bis(tricyclohexylphosphine)ruthenium(II). In specific embodiments of the processes described herein the cross-metathesis catalyst is [1,3-bis(2,4,6-trimethylphenyl)-2-imidazolidinylidene]dichloro(3-methyl-2-butenylidene)(tricyclohexylphosphine)ruthenium(II). In some embodiments of the processes described herein the cross-metathesis catalyst is [1,3-bis(2,4,6-trimethylphenyl)-2-imidazolidinylidene]dichloro(benzylidene)bis(3-bromopyridine)ruthenium(II).

In additional embodiments of the processes described herein the reaction is conducted at a temperature of about 15° C. to about 35° C. In other embodiments of the processes described herein the reaction is conducted at a temperature of about 20° C. to about 150° C. with thermal heating. In some embodiments of the processes described herein the reaction is conducted at a temperature of about 20° C. to about 150° C. with microwave irradiation heating. In some embodiments of the processes described herein the amount of cross-metathesis catalyst is between about 1 mol % and about 40 mol %. In other embodiments of the processes described herein the amount of cross-metathesis catalyst is between about 20 mol % and about 30 mol %. In some embodiments of the processes described herein the amount of cross-metathesis catalyst is between about 1 mol % and about 10 mol %. In other embodiments of the processes described herein the amount of cross-metathesis catalyst is between about 10 mol % and about 20 mol %. In some embodiments of the processes described herein the amount of cross-metathesis catalyst is between about 20 mol % and about 25 mol %. In other embodiments of the processes described herein the amount of cross-metathesis catalyst is between about 25 mol % and about 30 mol %.

Provided herein are pharmaceutical compositions comprising a compound of Formula (V) or a pharmaceutically acceptable salt, prodrug, solvate, polymorph, tautomer or isomer thereof. In various embodiments, the pharmaceutical composition comprises at least one pharmaceutically acceptable carrier.

In a one embodiment, the disclosure provides for compounds of Formula (V) and their pharmaceutically acceptable salts. In further or additional embodiments, the disclosure provides for compounds of Formula (V) and their pharmaceutically acceptable solvates. In further or additional embodiments, the disclosure provides for compounds of Formula (V) and their pharmaceutically acceptable polymorphs. In further or additional embodiments, the disclosure provides for compounds of Formula (V) and their pharmaceutically acceptable esters. In further or additional embodiments, the disclosure provides for compounds of Formula (V) and their pharmaceutically acceptable tautomers. In further or additional embodiments, the disclosure provides for compounds of Formula (V) and their pharmaceutically acceptable prodrugs.

### Salts

In some embodiments, the compounds described herein also exist as their pharmaceutically acceptable salts, which in other embodiments are useful for treating disorders. For example, the disclosure provides for methods of treating diseases, by administering pharmaceutically acceptable salts of the compounds described herein. In some embodiments, the pharmaceutically acceptable salts are administered as pharmaceutical compositions.

Thus, in some embodiments, the compounds described herein are prepared as pharmaceutically acceptable salts formed when an acidic proton present in the parent compound either is replaced by a metal ion, for example an alkali metal ion, an alkaline earth ion, or an aluminum ion; or coordinates with an organic base. In other embodiments, base addition salts are also prepared by reacting the free acid form of the compounds described herein with a pharmaceutically acceptable inorganic or organic base, including, but not limited to organic bases such as ethanolamine, diethanolamine, triethanolamine, tromethamine, N-methylglucamine, and the like and inorganic bases such as aluminum hydroxide, calcium hydroxide, potassium hydroxide, sodium carbonate, sodium hydroxide, and the like. In addition, in further embodiments, the salt forms of the disclosed compounds are prepared using salts of the starting materials or intermediates.

Further, in some embodiments, the compounds described herein are prepared as pharmaceutically acceptable salts formed by reacting the free base form of the compound with a pharmaceutically acceptable inorganic or organic acid, including, but not limited to, inorganic acids such as hydrochloric acid, hydrobromic acid, sulfuric acid, nitric acid, phosphoric acid metaphosphoric acid, and the like; and organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, Q-toluenesulfonic acid, tartaric acid, trifluoroacetic acid, citric acid, benzoic acid, 3-(4-hydroxybenzoyl)benzoic acid, cinnamic acid, mandelic acid, arylsulfonic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, and muconic acid.

Pharmaceutically acceptable salts are generally known, and in other embodiments include, by way of example but not limitation, acetate, benzenesulfonate, besylate, benzoate, bicarbonate, bitartrate, bromide, calcium edetate, carnsylate, carbonate, citrate, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, mucate, napsylate, nitrate, pamoate (embonate), pantothenate, phosphate/diphosphate, polygalacturonate, salicylate, stearate, subacetate, succinate, sulfate, tannate, tartrate, or teoclate. In further embodiments, other pharmaceutically acceptable salts are found in, for example, Remington: The Science and Practice of Pharmacy (20^{th} ed.) Lippincott, Williams & Wilkins (2000). In some embodiments, pharmaceutically acceptable salts include, for example, acetate, benzoate, bromide, carbonate, citrate, gluconate, hydrobromide, hydrochloride, maleate, mesylate, napsylate, pamoate (embonate), phosphate, salicylate, succinate, sulfate, or tartrate.

### Solvates

In other embodiments, the compounds described herein also exist in various solvated forms, which in further embodiments are useful for treating disorders. For example, the disclosure provides for methods of treating diseases, by administering solvates of the compounds described herein. In some embodiments, the solvates are administered as pharmaceutical compositions. In other embodiments, the solvates are pharmaceutically acceptable solvates.

Solvates contain either stoichiometric or non-stoichiometric amounts of a solvent, and in further embodiments are formed during the process of crystallization with pharmaceutically acceptable solvents such as water, ethanol, and the like. Hydrates are formed when the solvent is water, or alcoholates are formed when the solvent is alcohol. In some embodiments, solvates of the compounds described herein are conveniently prepared or formed during the processes described herein. By way of example only, in some embodiments, hydrates of the compounds described herein are conveniently prepared by recrystallization from an aqueous/organic solvent mixture, using organic solvents including, but not limited to, dioxane, tetrahydrofuran or methanol. In addition, in other embodiments, the compounds provided herein exist in unsolvated as well as solvated forms. In general, the solvated forms are considered equivalent to the unsolvated forms for the purposes of the compounds and methods provided herein.

### Polymorphs

In some embodiments, the compounds described herein also exist in various polymorphic states, all of which are herein contemplated, and in other embodiments, are useful for treating disorders. For example, the disclosure provides for methods of treating diseases, by administering polymorphs of the compounds described herein. In some embodiments, the various polymorphs are administered as pharmaceutical compositions.

Thus, the compounds described herein include all their crystalline forms, known as polymorphs. Polymorphs include the different crystal packing arrangements of the same elemental composition of the compound. In some embodiments, polymorphs have different X-ray diffraction patterns, infrared spectra, melting points, density, hardness, crystal shape, optical and electrical properties, stability, solvates and solubility. In other embodiments, various factors such as the recrystallization solvent, rate of crystallization, and storage temperature cause a single crystal form to dominate.

### Prodrugs

In some embodiments, the compounds described herein also exist in prodrug form, which in other embodiments, are useful for treating disorders. For example, the disclosure provides for methods of treating diseases, by administering prodrugs of the compounds described herein. In some embodiments, the prodrugs are administered as pharmaceutical compositions.

Prodrugs are generally drug precursors that, following administration to a subject and subsequent absorption, are converted to an active, or a more active species via some process, such as conversion by a metabolic pathway. Some prodrugs have a chemical group present on the prodrug that renders it less active and/or confers solubility or some other property to the drug. Once the chemical group has been cleaved and/or modified from the prodrug the active drug is generated. Prodrugs are often useful because, in some embodiments, they are easier to administer than the parent drug. In further embodiments, they are bioavailable by oral administration whereas the parent is not. In some embodiments, the prodrug has improved solubility in pharmaceutical compositions over the parent drug. An example, without limitation, of a prodrug would be the compound as described herein which is administered as an ester (the “prodrug”) to facilitate transmittal across a cell membrane where water solubility is detrimental to mobility but which then is metabolically hydrolyzed to the carboxylic acid, the active entity, once inside the cell where water-solubility is beneficial. In some embodiments, the prodrug is a short peptide (polyamino acid) bonded to an acid group where the peptide is metabolized to reveal the active moiety.

In other embodiments, prodrugs are designed as reversible drug derivatives, for use as modifiers to enhance drug transport to site-specific tissues. The design of prodrugs to date has been to increase the effective water solubility of the therapeutic compound for targeting to regions where water is the principal solvent. See, e.g., Fedorak et al., Am. J. Physiol., 269:G210-218 (1995); McLoed et al., Gastroenterol, 106:405-413 (1994); Hochhaus et al., Biomed. Chrom., 6:283-286 (1992); J. Larsen and H. Bundgaard, Int. J. Pharmaceutics, 37, 87 (1987); J. Larsen et al., Int. J. Pharmaceutics, 47, 103 (1988); Sinkula et al., J. Pharm. Sci., 64:181-210 (1975); T. Higuchi and V. Stella, Prodrugs as Novel Delivery Systems, Vol. 14 of the A.C.S. Symposium Series; and Edward B. Roche, Bioreversible Carriers in Drug Design, American Pharmaceutical Association and Pergamon Press, 1987, all incorporated herein in their entirety.

Pharmaceutically acceptable prodrugs of the compounds described herein include, but are not limited to, esters, carbonates, thiocarbonates, N-acyl derivatives, N-acyloxyalkyl derivatives, quaternary derivatives of tertiary amines, N-Mannich bases, Schiff bases, amino acid conjugates, phosphate esters, metal salts and sulfonate esters. Various forms of prodrugs are known. See for example Design of Prodrugs, Bundgaard, A. Ed., Elseview, 1985 and Method in Enzymology, Widder, K. et al., Ed.; Academic, 1985, vol. 42, p. 309-396; Bundgaard, H. “Design and Application of Prodrugs” in A Textbook of Drug Design and Development, Krosgaard-Larsen and H. Bundgaard, Ed., 1991, Chapter 5, p. 113-191; and Bundgaard, H., Advanced Drug Delivery Review, 1992, 8, 1-38, each of which is incorporated herein by reference. The prodrugs described herein include, but are not limited to, the following groups and combinations of these groups; amine derived prodrugs:

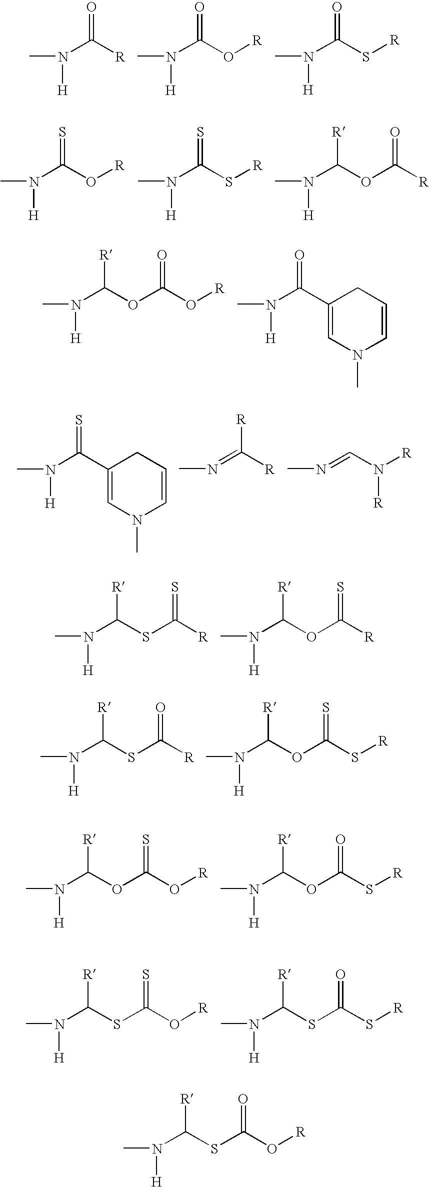

Hydroxy prodrugs include, but are not limited to acyloxyalkyl esters, alkoxycarbonyloxyalkyl esters, alkyl esters, aryl esters and disulfide containing esters.

In some embodiments, prodrugs include compounds wherein an amino acid residue, or a polypeptide chain of two or more (e. g., two, three or four) amino acid residues is covalently joined through an amide or ester bond to a free amino, hydroxy or carboxylic acid group of compounds of the present disclosure. The amino acid residues include but are not limited to the 20 naturally occurring amino acids commonly designated by three letter symbols and also includes 4-hydroxyproline, hydroxylysine, demosine, isodemosine, 3-methylhistidine, norvaline, beta-alanine, gamma-aminobutyric acid, cirtulline, homocysteine, homoserine, ornithine and methionine sulfone. Additional types of prodrugs are also encompassed.

Prodrug derivatives of compounds described herein can be prepared by methods described herein (e.g., for further details see Saulnier et al., (1994), Bioorganic and Medicinal Chemistry Letters, Vol. 4, p. 1985). By way of example only, in some embodiments, appropriate prodrugs are prepared by reacting a non-derivatized compound of Formula (V) with a suitable carbamylating agent, such as, but not limited to, 1,1-acyloxyalkylcarbanochloridate, para-nitrophenyl carbonate, or the like. Prodrug forms of the herein described compounds, wherein the prodrug is metabolized in vivo to produce a derivative as set forth herein are included within the scope of the claims. Indeed, in some embodiments, some of the herein-described compounds are a prodrug for another derivative or active compound.

In some embodiments, compounds of Formula (V) having free amino, amido, hydroxy or carboxylic groups are converted into prodrugs. For instance, in some embodiments, free carboxyl groups are derivatized as amides or alkyl esters. In other embodiments, free hydroxy groups are derivatized using groups including but not limited to hemisuccinates, phosphate esters, dimethylaminoacetates, and phosphoryloxymethyloxycarbonyls, as outlined in Advanced Drug Delivery Reviews 1996, 19, 115. Carbamate prodrugs of hydroxy and amino groups are also included, as are carbonate prodrugs, sulfonate esters and sulfate esters of hydroxy groups.

Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med. Chem. 1996, 39, 10. In some embodiments, free amines are derivatized as amides, sulfonamides or phosphonamides. In some embodiments, all of these prodrug moieties incorporate groups including but not limited to ether, amine and carboxylic acid functionalities. In other embodiments, phosphate ester functionalities are used as prodrug moieties.

In some other embodiments, sites on the aromatic ring portions of the compounds described herein are susceptible to various metabolic reactions, therefore incorporation of appropriate substituents on the aromatic ring structures, reduces, minimizes or eliminates this metabolic pathway.

### Methods of Treatment

In general, methods of using the compounds described herein comprise administering to a subject in need thereof a therapeutically effective amount of a compound presented herein. Diseases that may be treated with the compounds of the presently described are those that are characterized by cellular hyperproliferation, such as cancers, tumors, and inflammatory disorders.

In one embodiment, the compounds are useful for treating cancer, including leukemia, and other diseases or disorders involving abnormal cell proliferation, myeloproliferative disorders, hematological disorders, asthma, or inflammatory diseases.

More specific examples of cancers treated with the compounds of the disclosure include breast cancer, lung cancer, melanoma, colorectal cancer, bladder cancer, ovarian cancer, prostate cancer, renal cancer, squamous cell cancer, glioblastoma, mesothelioma or small cell lung cancer, pancreatic cancer, leiomyosarcoma, multiple myeloma, papillary renal cell carcinoma, gastric cancer, liver cancer, head and neck cancer, melanoma, glioblastoma, cancers of oral cavity and pharynx, cancers of the respiratory system, cancers of bones and joints, cancers of soft tissue, cancers of the eye and orbit, cancers of the nervous system, cancers of the lymphatic system, cancers of the endocrine system, heart cancer, plasmacytomas, retinoblastoma, synovioma, rhabdomyosarcoma, alveolar soft part sarcoma, or leukemia (e.g. myeloid, chronic myeloid, acute lymphoblastic, chronic lymphoblastic, Hodgkins, non-hodgkin's lymphoma, follicular lymphoma, pre-B acute leukemia, chronic lymphocytic B-leukemia, mesothelioma or small cell lung cancer, and other leukemias and hematological cancers).

In some embodiments, the cancer is a solid tumor. Solid tumors can be classified by the type of cells forming the solid tumor, such as sarcomas, carcinomas, and lymphomas. Solid tumors can also be classified by organ site. Examples of solid tumors classified by organ site include head and neck tumors, lung tumors, skin tumors, esophagus tumors, gastric tumors, pancreas tumors, colorectal tumors, prostate tumors, sarcoma tumors, melanoma tumors, breast tumors, cervix tumors, endometrial tumors, ovarian tumors, liver tumors, biliary gall bladder tumors, small bowel tumors, and anus tumors.

In some embodiments, the compositions and methods described herein are used to treat hematologic cancers. Hematologic cancers include leukemia, lymphoma, myeloma, myelodysplastic syndrome, and myeloproliferative disorders. Leukemia includes acute leukemia, acute myelogenous leukemia (AML), acute lymphocytic leukemia (ALL), acute nonlymphocytic leukemia (ANLL), chronic myelogenous leukemia (CML), and chronic lymphocytic leukemia (CLL). Lymphoma includes Hodgkin lymphoma, non-Hodgkin lymphoma, cutaneous t-cell lymphoma (CTCL) (both granulocytic and monocytic), and mantle cell lymphoma (MCL). Myeloma includes multiple myeloma, extramedullary plasmacytoma, solitary myeloma, and indolent myeloma. Myelodysplastic syndrome includes clonal anemia, clonal sideroblastic anemia, clonal pancytopenia, and oligoblastic myelogenous leukemia. Myeloproliferative disorders include polycythemia vera, primary thrombocythemia, and idiopathic myelofibrosis.

In some embodiments, the compositions and methods described herein are used to treat cancers of epithelial origin. Generally, cancers having an epithelial origin include squamous cell carcinoma, adenocarcinoma, and transitional cell carcinoma. Specifically, non-limiting examples of premalignant or precancerous cancers/tumors having epithelial origin include actinic keratoses, arsenic keratoses, xeroderma pigmentosum, Bowen's disease, leukoplakias, metaplasias, dysplasias and papillomas of mucous membranes, e.g. of the mouth, tongue, pharynx and larynx, precancerous changes of the bronchial mucous membrane such as metaplasias and dysplasias (especially frequent in heavy smokers and people who work with asbestos and/or uranium), dysplasias and leukoplakias of the cervix uteri, vulval dystrophy, precancerous changes of the bladder, e.g. metaplasias and dysplasias, papillomas of the bladder as well as polyps of the intestinal tract. Non-limiting examples of semi-malignant or malignant cancers/tumors of the epithelial origin are breast cancer, skin cancer (e.g., basal cell carcinomas), bladder cancer (e.g., superficial bladder carcinomas), colon cancer, gastro-intestinal (GI) cancer, prostate cancer, uterine cancer, cervical cancer, ovarian cancer, esophageal cancer, stomach cancer, laryngeal cancer and lung cancer.

More examples of cancers of epithelial origin include adenocarcinoma, basal cell carcinoma, choriocarcinoma, cystadenocarcinoma, embryonal carcinoma, epithelial carcinoma, hepatocellular carcinoma, hepatoma, large cell carcinoma, medullary thyroid carcinoma, papillary carcinoma, papillary adenocarcinomas, sebaceous gland carcinoma, small cell lung carcinoma, squamous cell carcinoma, and sweat gland carcinoma.

In some embodiments, the compositions and methods described herein are used to treat cancers of the central nervous system. Cancers of the central nervous system include hemangioblastoma, medulloblastoma, meningioma, and neuroblastoma.

In some embodiments, the compositions and methods described herein are used to treat genital cancers. Gynecological cancers include cervical cancer, endometrial cancer, ovarian cancer, uterine cancer, vaginal cancer, uveal melanoma, and vulvar cancer. Male genital cancer includes prostate cancer, testicular cancer, seminoma, and penile cancer.

In some embodiments, the compositions and methods described herein are used to treat oral cancers. Oral cancers include cancers of the oral cavity, and cancers of the oropharynx.

In some embodiments, the compositions and methods described herein are used to treat skin cancers. Skin cancers include basal cell carcinoma, squamous cell carcinoma, Kaposi's sarcoma, merkel cell cancer, actinic keratosis, melanoma, cutaneous melanoma, and other non-epithelial skin cancers.

In some embodiments, the compositions and methods described herein are used to treat genitourinary cancers. Genitourinary cancers include bladder cancer, renal cell cancer, adrenocortical carcinoma, prostate cancer, testicular cancer, penile cancer, renal pelvis cancer, and urethral cancer.

In some embodiments, the compositions and methods described herein are used to treat endocrine cancers. Endocrine cancers include thyroid cancer, adrenocortical cancer, neuroblastoma, pheochromocytoma, pinealoma, and parathyroid cancer.

In some embodiments, the compositions and methods described herein are used to treat kidney cancers. Kidney cancers include kidney cancer, renal cell carcinoma (RCC), Wilm's tumor, clear cell carcinoma, papillary renal cell carcinoma, and pelvic renal cancer.

In some embodiments, the compositions and methods described herein are used to treat thoracic cancers. Thoracic cancers include non-small cell lung cancer (NSCLC), small cell lung cancer (SCLC), mesothelioma, EGFR-inhibitor-resistant NSCLC, esophageal cancer, bronchial cancer, mesothelioma, and other cancers of the respiratory organs.

In some embodiments, the compositions and methods described herein are used to treat gastrointestinal tract cancers. Gastrointestinal tract cancers include gastrointestinal stromal tumors (GIST), esophageal cancer, gastric cancer, hepatocellular carcinoma (HCC), gallbladder cancer, pancreatic cancer, colorectal cancer, anal cancer, anorectal cancer, liver cancer, intrahepatic bile duct cancer, extrahepatic bile duct cancer, small intestine cancer, and other biliary or digestive organ cancers.

In some embodiments, the compositions and methods described herein are used to treat brain cancers. Brain cancers can be classified by the type of cells where the cancer originates. Brain cancers that originate in the glial cells (gliomas or glioblastomas) include astrocytoma, anaplastic astrocytoma, glioblastoma multiforme, brain stem gliomas, ependymomas, blastoglioma, optic nerve gliomas, ependymoma, and oligodendrogliomas. Brain cancers that originate in the schwann cells (schwannomas) include acoustic neuroma. Brain cancers that originate in cells other than the glial cells include medulloblastomas, meningiomas, schwannomas, craniopharyngiomas, germ cell tumors, chordoma, craniopharyngioma, and pineal region tumors.

In some embodiments, the compositions and methods described herein are used to treat sarcomas. Sarcomas include leiomyosarcoma, angiosarcoma, chondrosarcoma, endotheliosarcoma, fibrosarcoma, Kaposi's sarcoma, liposarcoma, lymphangiosarcoma, lymphangioendotheliosarcoma, myxosarcoma, neurofibrosarcoma, osteogenic sarcoma, rhabdomyosarcoma, hepatoma, chandro sarcoma, fibrosarcoma, myxofibrosacroma, desmoid tumors, synovial sarcomas, malignant peripheral nerve sheet tumors (MPNST), gastrointestinal stromal tumors (GIST), and Ewing's tumors. Sarcomas can be classified as soft tissue sarcomas, osteosarcomas, and chondrosarcomas. Soft tissue sarcomas include fibrosarcoma, myxofibrosarcoma, desmoid tumors, liposarcoma, synovial sarcoma, rhabdomyosarcoma, leiomyosarcoma, malignant peripheral nerve sheet tumors (MPNST), gastrointestinal stromal tumors (GIST), angiosarcoma, Kaposi's sarcoma, and Ewing's tumors. Osteosarcomas include Ewing's tumors. Chondrosarcomas include central chondrosarcoma, peripheral chondrosarcoma, de-differentiated chondrosarcoma, clear cell chondrosarcoma, mesenchymal chondrosarcoma, and juxtacortical chondrosarcoma.

In some embodiments, the compositions and methods described herein are used to treat pancreatic cancers. Pancreatic cancer includes adenocarcinoma of the pancreas, and cystadenocarcinoma.

In some embodiments, the compositions and methods described herein are used to treat head and neck cancer. Head and neck cancer include laryngeal cancer, oropharyngeal cancer, parathyroid cancer, thyroid cancer, oral cancer, nasopharyngeal cancer, nasal cavity and paranasal sinus cancers, esophageal cancer, and hypopharyngeal cancer.

Other specific examples of diseases or disorders for which treatment by the compounds or compositions of the disclosure are useful for treatment or prevention include, but are not limited to transplant rejection (for example, kidney, liver, heart, lung, islet cells, pancreas, bone marrow, cornea, small bowel, skin allografts or xenografts and other transplants), graft vs. host disease, osteoarthritis, rheumatoid arthritis, multiple sclerosis, diabetes, diabetic retinopathy, inflammatory bowel disease (for example, Crohn's disease, ulcerative colitis, and other bowel diseases), renal disease, cachexia, septic shock, lupus, myasthenia gravis, psoriasis, dermatitis, eczema, seborrhea, Alzheimer's disease, Parkinson's disease, stem cell protection during chemotherapy, ex vivo selection or ex vivo purging for autologous or allogeneic bone marrow transplantation, ocular disease, retinopathies (for example, macular degeneration, diabetic retinopathy, and other retinopathies), corneal disease, glaucoma, infections (for example bacterial, viral, or fungal), heart disease, including, but not limited to, restenosis.

Illustrative examples of inflammatory disorders include, for example, atrophic gastritis, inflammatory hemolytic anemia, graft rejection, inflammatory neutropenia, bullous pemphigoid, coeliac disease, demyelinating neuropathies, dermatomyositis, inflammatory bowel disease (ulcerative colitis and Crohn's disease), multiple sclerosis, myocarditis, myositis, nasal polyps, chronic sinusitis, pemphigus vulgaris, primary glomerulonephritis, psoriasis, surgical adhesions, stenosis or restenosis, scleritis, scleroderma, eczema (including atopic dermatitis. irritant dermatitis, allergic dermatitis), periodontal disease (i.e., periodontitis), polycystic kidney disease, and type I diabetes.

Other examples of inflammatory diseases include vasculitis (e.g., Giant cell arteritis (temporal arteritis, Takayasu's arteritis), polyarteritis nodosa, allergic angiitis and granulomatosis (Churg-Strauss disease), polyangitis overlap syndrome, hypersensitivity vasculitis (Henoch-Schonlein purpura), serum sickness, drug-induced vasculitis, infectious vasculitis, neoplastic vasculitis, vasculitis associated with connective tissue disorders, vasculitis associated with congenital deficiencies of the complement system, Wegener's granulomatosis, Kawasaki's disease, vasculitis of the central nervous system, Buerger's disease and systemic sclerosis); gastrointestinal tract diseases (e.g., pancreatitis, Crohn's disease, ulcerative colitis, ulcerative proctitis, primary sclerosing cholangitis, benign strictures of any cause including ideopathic (e.g., strictures of bile ducts, esophagus, duodenum, small bowel or colon); respiratory tract diseases (e.g., asthma, hypersensitivity pneumonitis, asbestosis, silicosis and other forms of pneumoconiosis, chronic bronchitis and chronic obstructive airway disease); nasolacrimal duct diseases (e.g., strictures of all causes including ideopathic); and eustachean tube diseases (e.g., strictures of all causes including ideopathic).

Because the use of microtubule-stabilizing agents such as the laulimalides, taxanes, epothilones, discodermolide, eleutherobin, and the like, to treat inflammatory disorders are not as well documented as the use of microtubule-stabilizing agents to treat cancers and tumors, three representative examples of inflammatory disorders are discussed in greater detail below.

### Psoriasis and Eczema

Utilizing the agents, compositions and methods provided herein, a wide variety of inflammatory skin diseases can be readily treated or prevented. For example, within one embodiment an inflammatory skin disease such as psoriasis or eczema is treated or prevented by delivering to a site of inflammation (or a potential site of inflammation) an agent described herein. Briefly, skin cells are genetically programmed to follow two possible programs—normal growth or wound healing. In the normal growth pattern, skin cells are created in the basal cell layer and then move up through the epidermis to the skin surface. Dead cells are shed from healthy skin at the same rate new cells are created. The turnover time (i.e., time from cell birth to death) for normal skin cells is approximately 28 days. During wound healing, accelerated growth and repair is triggered resulting in rapid turnover of skin cells (to replace and repair the wound), increased blood supply (to meet the increased metabolic needs associated with growth) and localized inflammation.

In many respects, psoriasis is similar to an exaggerated wound healing process where skin cells (called “keratinocytes”) are created and pushed to the skin surface in as little as 2-4 days. Psoriasis occurs when skin cells hyperproliferate and the surface skin cannot shed the dead cells fast enough. The excess keratinocytes build up and form elevated, scaly lesions. This growth is supported by new blood vessels in the dermis (the support tissue beneath the epidermis) that are established to provide the nutrients necessary to support the hyperproliferating keratinocytes. At the same time, lymphocytes, neutrophils and macrophage invade the tissue, creating inflammation, swelling and soreness, and potentially producing growth factors that augment the rapid proliferation of the keratinocytes. All these cells (keratinocytes, vascular endothelial cells and white blood cells) produce tissue degrading enzymes or proteinases that aid in the progression of the disease and the destruction of surrounding tissue.

Utilizing the compositions described herein, in some embodiments, inflammatory skin lesions are readily treated. In other embodiments, the compounds presented herein are administered directly to the site of inflammation (or a potential site of inflammation), in order to treat or prevent the disease. In another embodiment, the one or more agents are delivered as a composition along with a polymeric carrier, or in a liposome, cream or ointment formulation as discussed previously. Within other embodiments, the agents or compositions are delivered either topically, or by subcutaneous administration. An effective therapy for psoriasis will achieve at least one of the following: decrease the number and severity of skin lesions, decrease the frequency or duration of active disease exacerbations, increase the amount of time spent in remission (i.e., periods when the patient is symptom-free) and/or decrease the severity or duration of associated symptoms (e.g., joint pain and swelling, axial skeletal pain, bowel symptoms). Clinically the treatment will result in a reduction in the size or number of skin lesions, diminution of cutaneous symptoms (pain, burning and bleeding of the affected skin) and/or a reduction in associated symptoms (e.g., joint redness, heat, swelling, diarrhea. abdominal pain). Pathologically, the compounds presented herein will produce at least one of the following: inhibition of keratinocyte proliferation, reduction of skin inflammation (for example, by impacting on: attraction and growth factors, antigen presentation, production of reactive oxygen species and matrix metalloproteinases), and inhibition of dermal angiogenesis.

In further embodiments, the compounds described herein are administered in any manner sufficient to achieve the above end points, and in some embodiments methods include topical and systemic administration. In further embodiments, patients with localized disease are administered a topical cream, ointment or emollient applied directly to the psoriatic lesions. For example, a topical cream containing about 0.001% to about 10% of a compound described herein by weight is administered depending upon severity of the disease and the patient's response to treatment. In another embodiment, a topical preparation containing a compound presented herein at about 0.01% to about 1% by weight is administered to psoriatic lesions. In a further embodiment, direct intracutaneous injection of a compound described herein in a suitable pharmaceutical vehicle is used for the management of individual lesions. In other embodiments, where patients with widespread disease or extracutaneous symptoms (e.g., psoriatic arthritis, Reiter's syndrome, associated spondylitis, associated inflammatory bowel disease), systemic treatment is administered. For example, in other embodiments intermittent treatments with an intravenous formulation is administered at a dose of about 10 to about 75 mg/m² of a compound described herein depending upon therapeutic response and patient tolerance. An equivalent oral preparation would also be suitable for this indication.

In other embodiments, other dermatological conditions that also benefit from topical agents comprising compounds presented herein include: eczematous disease (atopic dermatitis. contact dermatitis, eczema), immunobullous disease, pre-malignant epithelial tumors, basal cell carcinoma, squamous cell carcinoma, keratocanthoma, malignant melanoma and viral warts. In further embodiments, topical creams, ointments, and emollients containing about 0.001% to about 10% of a compound described herein by weight are suitable for the management of these conditions.

### Multiple Sclerosis

In some embodiments, compounds presented herein are utilized to treat or prevent chronic inflammatory neurological disorders, such as multiple sclerosis. Briefly, multiple sclerosis (“MS”) is a devastating demyelinating disease of the human central nervous system. Although its etiology and pathogenesis is not known, genetic, immunological and environmental factors are believed to play a role. In the course of the disease, there is a progressive demyelination in the brain of MS patients resulting in the loss of motor function. Although the exact mechanisms involved in the loss of myelin are not understood, there is an increase in astrocyte proliferation and accumulation in the areas of myelin destruction. At these sites, there is macrophage-like activity and increased protease activity which is at least partially responsible for degradation of the myelin sheath.

In other embodiments, compounds presented herein are administered to the site of inflammation (or a potential site of inflammation), in order to treat or prevent the disease. In further embodiments, such agents, are delivered as a composition along with a polymeric carrier, or in a liposome formulation as previously. Within other embodiments, the agents or compositions are administered orally, intravenously, or by direct administration (in other embodiments with ultrasound, CT, fluoroscopic, MRI or endoscopic guidance) to the disease site. In yet other embodiments, an effective therapy for multiple sclerosis will accomplish one or more of the following: decrease the severity of symptoms; decrease the duration of disease exacerbations; increase the frequency and duration of disease remission/symptom-free periods; prevent fixed impairment and disability; and/or prevent/attenuate chronic progression of the disease. Clinically, this would result in improvement in visual symptoms (visual loss, diplopia), gait disorders (weakness, axial instability, sensory loss, spasticity, hyperreflexia, loss of dexterity), upper extremity dysfunction (weakness, spasticity, sensory loss), bladder dysfunction (urgency, incontinence, hesitancy, incomplete emptying), depression, emotional lability, and cognitive impairment. Pathologically, in some embodiments, the treatment reduces one or more of the following, such as myelin loss, breakdown of the blood-brain barrier, perivascular infiltration of mononuclear cells, immunologic abnormalities, gliotic scar formation and astrocyte proliferation, metalloproteinase production, and impaired conduction velocity.

In other embodiments, the agents described herein are administered in any manner sufficient to achieve the above endpoints. However, in further embodiments methods of administration include intravenous, oral, or subcutaneous, intramuscular or intrathecal injection. In some embodiments, the microtubule-stabilizing agent is administered as a chronic low dose therapy to prevent disease progression, prolong disease remission or decrease symptoms in active disease. In some other embodiments, the therapeutic agent is administered in higher doses as a “pulse” therapy to induce remission in acutely active disease. In yet further embodiments, the minimum dose capable of achieving these endpoints is used and varies according to patient, severity of disease, formulation of the administered agent, and route of administration. For example, in some embodiments, includes about 10 to about 75 mg/m² of a compound described herein once every 1 to 4 weeks, about 10 to about 75 mg/m² daily, as tolerated, or about 10 to about 175 mg/m² once weekly, as tolerated or until symptoms subside.

### Arthritis

Inflammatory arthritis is a serious health problem in developed countries, particularly given the increasing number of aged individuals. For example, one form of inflammatory arthritis, rheumatoid arthritis (“RA”) is a multisystem chronic, relapsing, inflammatory disease of unknown cause. Although many organs can be affected, RA is basically a severe form of chronic synovitis that sometimes leads to destruction and ankyiosis of affected joints (Robbins Pathological Basis of Disease, by R. S. Cotran, V. Kumar, and S. L. Robbins, W. B. Saunders Co., 1989). Pathologically, the disease is characterized by a marked thickening of the synovial membrane which forms villous projections that extend into the joint space, multilayering of the synoviocyte lining (synoviocyte proliferation), infiltration of the synovial membrane with white blood cells (macrophages, lymphocytes, plasma cells, and lymphoid follicles; called an “inflammatory synovitis”), and deposition of fibrin with cellular necrosis within the synovium. The tissue formed as a result of this process is called pannus and, eventually the pannus grows to fill the joint space. The pannus develops an extensive network of new blood vessels through the process of angiogenesis that is essential to the evolution of the synovitis. The release of digestive enzymes (matrix metalloproteinases such as collagenase, stromelysin, and the like) and other mediators of the inflammatory process (e.g., hydrogen peroxide, superoxides, lysosomal enzymes, and products of arachadonic acid metabolism) from the cells of the pannus tissue leads to the progressive destruction of the cartilage tissue. The pannus invades the articular cartilage leading to erosions and fragmentation of the cartilage tissue. Eventually there is erosion of the subchondral bone with fibrous ankylosis and ultimately bony ankylosis, of the involved joint. It is generally believed, but not conclusively proven, that RA is an autoimmune disease, and that many different arthrogenic stimuli activate the immune response in the immunogenetically susceptible host. Both exogenous infectious agents (Ebstein-Barr virus, rubella virus, cytomegalovirus, herpes virus, human T-cell lymphotropic virus, Mycoplasma, and others) and endogenous proteins (collagen, proteoglycans, altered immunoglobulins) have been implicated as the causative agent that triggers an inappropriate host immune response. Regardless of the inciting agent, autoimmunity plays a role in the progression of the disease. In particular, the relevant antigen is ingested by antigen-presenting cells (macrophages or dendritic cells in the synovial membrane), processed, and presented to T lymphocytes. The T cells initiate a cellular immune response and stimulate the proliferation and differentiation of B lymphocytes into plasma cells. The end result is the production of an excessive inappropriate immune response directed against the host tissues (e.g., antibodies directed against type II collagen, antibodies directed against the Fc portion of autologous IgG (called “Rheumatoid Factor”)). This further amplifies the immune response and hastens the destruction of the cartilage tissue. Once this cascade is initiated numerous mediators of cartilage destruction are responsible for the progression of rheumatoid arthritis.

Thus, within one aspect described herein, methods are provided for treating or preventing inflammatory arthritis (e.g., rheumatoid arthritis) comprising the step of administering to a patient a therapeutically effective amount of a microtubule-stabilizing agent described herein. Inflammatory arthritis includes a variety of conditions including, but not limited to, rheumatoid arthritis, systemic lupus erythematosus, systemic sclerosis (scleroderma), mixed connective tissue disease, Sjogren's syndrome, ankylosing spondylitis, Behcet's syndrome, sarcoidosis, and osteoarthritis—all of which feature inflamed, painful joints as a prominent symptom. Within some embodiments, compounds described herein are administered directly to a joint by intra-articular injection, as a surgical paste or administered by another route, e.g., systemically or orally. Such agents, within other embodiments, are delivered as a composition along with a polymeric carrier, or in a liposome formulation as discussed previously.

In some embodiments, an effective microtubule-stabilizing therapy for inflammatory arthritis will accomplish one or more of the following: (i) decrease the severity of symptoms (pain, swelling and tenderness of affected joints; morning stiffness. weakness, fatigue. anorexia, weight loss); (ii) decrease the severity of clinical signs of the disease (thickening of the joint capsule, synovial hypertrophy, joint effusion, soft tissue contractures, decreased range of motion, ankylosis and fixed joint deformity); (iii) decrease the extra-articular manifestations of the disease (rheumatic nodules, vasculitis, pulmonary nodules, interstitial fibrosis, pericarditis, episcleritis, iritis, Felty's syndrome, osteoporosis); (iv) increase the frequency and duration of disease remission/symptom-free periods; (v) prevent fixed impairment and disability; and/or (vi) prevent/attenuate chronic progression of the disease. Pathologically, in other embodiments, an effective therapy for inflammatory arthritis will produce at least one of the following: (i) decrease the inflammatory response, (ii) disrupt the activity of inflammatory cytokines (such as IL-I, TNFa, FGF, VEGF), (iii) inhibit synoviocyte proliferation, (iv) block matrix metalloproteinase activity, and/or (v) inhibit angiogenesis. In another embodiment, compounds described herein are administered systemically (orally, intravenously, or by intramuscular or subcutaneous injection) in the minimum dose to achieve the above mentioned results. For patients with only a small number of joints affected, or with disease more prominent in a limited number of joints, the microtubule-stabilizing agent can be directly injected (intra-articular injection) into the affected joints. In further embodiments, compounds described herein are administered in any manner sufficient to achieve the above endpoints. However, in some embodiments methods of administration include intravenous, oral, or subcutaneous, intramuscular or intra-articular injection. In yet further embodiments, compounds of Formula (V) are administered as a chronic low dose therapy to prevent disease progression, prolong disease remission, or decrease symptoms in active disease.

### Pharmaceutical Compositions and Administration

In some embodiments, administration of the compounds and compositions described herein are effected by any method that enables delivery of the compounds to the site of action. These methods include oral routes, intraduodenal routes, parenteral injection (including intravenous, subcutaneous, intraperitoneal, intramuscular, intravascular or infusion), topical, intrapulmonary, rectal administration, by implant, by a vascular stent impregnated with the compounds described herein. For example, in other embodiments, compounds described herein are administered locally to the area in need of treatment. In some other embodiments, this is achieved by, for example, but not limited to, local infusion during surgery, topical application, e.g., cream, ointment, injection, catheter, or implant, said implant made, e.g., out of a porous, non-porous, or gelatinous material, including membranes, such as sialastic membranes, or fibers. In some embodiments, the administration is by direct injection at the site (or former site) of a tumor or neoplastic or pre-neoplastic tissue. In some embodiments, formulation and administration techniques are employed with the compounds and methods of the present disclosure, e.g., as discussed in Goodman and Gilman, The Pharmacological Basis of Therapeutics, current ed.; Pergamon; and Remington's, Pharmaceutical Sciences (current edition), Mack Publishing Co., Easton, Pa.

In some embodiments, the formulations include those suitable for oral, parenteral (including subcutaneous, intradermal, intramuscular, intravenous, intraarticular, intramedullary, intracardiac, intrathecal, intraspinal, intracapsular, subcapsular, intraorbital, intratracheal, subcuticular, intraarticular, subarachnoid, and intrastemal), intraperitoneal, transmucosal, transdermal, rectal and topical (including dermal, buccal, sublingual, intranasal, intraocular, and vaginal) administration although in other embodiments the most suitable route depends upon for example the condition and disorder of the recipient. In yet other embodiments, the formulations are conveniently presented in unit dosage form. All methods include the step of bringing into association the compound of the subject disclosure or a pharmaceutically acceptable salt, ester, prodrug or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

In another aspect, the present disclosure provides a pharmaceutical composition including a compound of Formula (V) in admixture with a pharmaceutically acceptable excipient.

In some embodiments, in therapeutic and/or diagnostic applications, the compounds of the disclosure are formulated for a variety of modes of administration, including systemic and topical or localized administration. In further embodiments, techniques and formulations generally are found in Remington: The Science and Practice of Pharmacy (20^{th} ed.) Lippincott, Williams & Wilkins (2000).

According to another aspect, the disclosure provides pharmaceutical compositions including compounds of the formulas described herein, and a pharmaceutically acceptable carrier, adjuvant, or vehicle.

In some embodiments, depending on the specific conditions being treated, such agents are formulated into liquid or solid dosage forms and administered systemically or locally. In further embodiments, the agents are delivered, for example, in a timed- or sustained-low release forms. In further embodiments, techniques for formulation and administration are found in Remington: The Science and Practice of Pharmacy (20^{th} ed.) Lippincott, Williams & Wilkins (2000). In other embodiments, suitable routes include oral, buccal, by inhalation spray, sublingual, rectal, transdermal, vaginal, transmucosal, nasal or intestinal administration; parenteral delivery, including intramuscular, subcutaneous, intramedullary injections, as well as intrathecal, direct intraventricular, intravenous, intra-articullar, intra-sternal, intra-synovial, intra-hepatic, intralesional, intracranial, intraperitoneal, intranasal, or intraocular injections or other modes of delivery.

In other embodiments, for injection, the agents of the disclosure are formulated and diluted in aqueous solutions, such as in physiologically compatible buffers such as Hank's solution, Ringer's solution, or physiological saline buffer. For such transmucosal administration, penetrants appropriate to the barrier to be permeated are used in the formulation.

Use of pharmaceutically acceptable inert carriers to formulate the compounds herein disclosed for the practice of the disclosure into dosages suitable for systemic administration is within the scope of the disclosure. With proper choice of carrier and suitable manufacturing practice, in other embodiments, the compositions of the present disclosure, in particular, those formulated as solutions, are administered parenterally, such as by intravenous injection. In yet other embodiments, the compounds are formulated readily using pharmaceutically acceptable carriers into dosages suitable for oral administration. Such carriers enable the compounds of the disclosure to be formulated as tablets, pills, capsules, liquids, gels, syrups, slurries, suspensions and the like, for oral ingestion by a patient to be treated.

In other embodiments, for nasal or inhalation delivery, the agents of the disclosure are also formulated by methods known to those of skill in the art, and include, for example, but not limited to, examples of solubilizing, diluting, or dispersing substances such as, saline, preservatives, such as benzyl alcohol, absorption promoters, and fluorocarbons.

Pharmaceutical compositions suitable for use in the present disclosure include compositions wherein active ingredients are contained in an effective amount to achieve its intended purpose.

In addition to the active ingredients, in other embodiments, these pharmaceutical compositions contain suitable pharmaceutically acceptable carriers comprising excipients and auxiliaries which facilitate processing of the active compounds into preparations which are used pharmaceutically. In some embodiments, the preparations formulated for oral administration are in the form of tablets, dragees, capsules, or solutions.

In other embodiments, pharmaceutical preparations for oral use are obtained by combining the active compounds with solid excipients, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations, for example, maize starch, wheat starch, rice starch, potato starch, gelatin, gum tragacanth, methyl cellulose, hydroxypropylmethyl-cellulose, sodium carboxymethyl-cellulose (CMC), and/or polyvinylpyrrolidone (PVP: povidone). If desired, in some other embodiments, disintegrating agents are added, such as the cross-linked polyvinylpyrrolidone, agar, or alginic acid or a salt thereof such as sodium alginate.

Dragee cores are provided with suitable coatings. For this purpose, concentrated sugar solutions may be used, which in some embodiments optionally contain gum arabic, talc, polyvinylpyrrolidone, carbopol gel, polyethylene glycol (PEG), and/or titanium dioxide, lacquer solutions, and suitable organic solvents or solvent mixtures. In further embodiments, dye-stuffs or pigments are added to the tablets or dragee coatings for identification or to characterize different combinations of active compound doses.

In yet other embodiments, pharmaceutical preparations that are used orally include push-fit capsules made of gelatin, as well as soft, sealed capsules made of gelatin, and a plasticizer, such as glycerol or sorbitol. In some other embodiments, push-fit capsules contain the active ingredients in admixture with filler such as lactose, binders such as starches, and/or lubricants such as talc or magnesium stearate and, optionally, stabilizers. In other embodiments, with soft capsules, the active compounds are dissolved or suspended in suitable liquids, such as fatty oils, liquid paraffin, or liquid polyethylene glycols (PEGs). In addition, in other embodiments, stabilizers are added.

In some embodiments, pharmaceutical preparations are formulated as a depot preparation. In other embodiments, such long acting formulations are administered by implantation (for example subcutaneously or intramuscularly) or by intramuscular injection. Thus, for example in further embodiments, the compounds are formulated with suitable polymeric or hydrophobic materials (for example as an emulsion in an acceptable oil) or ion exchange resins, or as sparingly soluble derivatives, for example, as a sparingly soluble salt.

In some other embodiments, for buccal or sublingual administration, the compositions take the form of tablets, lozenges, pastilles, or gels formulated in conventional manner. In further embodiments, such compositions comprise the active ingredient in a flavored basis such as sucrose and acacia or tragacanth.

In yet other embodiments, pharmaceutical preparations are formulated in rectal compositions such as suppositories or retention enemas, e.g., containing conventional suppository bases such as cocoa butter, polyethylene glycol, or other glycerides.

In some other embodiments, pharmaceutical preparations are administered topically, that is by non-systemic administration. This includes the application of the compound of the present disclosure externally to the epidermis or the buccal cavity and the instillation of such the compound into the ear, eye and nose, such that the compound does not significantly enter the blood stream. In contrast, systemic administration refers to oral, intravenous, intraperitoneal and intramuscular administration.

Pharmaceutical preparations suitable for topical administration include liquid or semi-liquid preparations suitable for penetration through the skin to the site of inflammation such as gels, liniments, lotions, creams, ointments or pastes, suspensions, powders, solutions, spray, aerosol, oil, and drops suitable for administration to the eye, ear or nose. In other embodiments, a formulation comprises a patch or a dressing such as a bandage or adhesive plaster impregnated with active ingredients and optionally one or more excipients or diluents. In some other embodiments, the amount of active ingredient present in the topical formulation varies widely. In other embodiments, the active ingredient comprises, for topical administration, from about 0.001% to about 10% w/w, for instance from about 1% to about 2% by weight of the formulation. It may however comprise as much as about 10% w/w but in other embodiments will comprise less than about 5% w/w, in yet other embodiments from about 0.1% to about 1% w/w of the formulation.

Formulations suitable for topical administration in the mouth include losenges comprising the active ingredient in a flavored basis, usually sucrose and acacia or tragacanth; pastimes comprising the active ingredient in an inert basis such as gelatin and glycerin, or sucrose and acacia; and mouthwashes comprising the active ingredient in a suitable liquid carrier.

Formulations suitable for topical administration to the eye also include eye drops wherein the active ingredient is dissolved or suspended in a suitable carrier, especially an aqueous solvent for the active ingredient.

Pharmaceutical preparations for administration by inhalation are conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. In some embodiments, for administration by inhalation or insufflation, pharmaceutical preparations take the form of a dry powder composition, for example a powder mix of the compound and a suitable powder base such as lactose or starch. In other embodiments, the powder composition is presented in unit dosage form, in for example, capsules, cartridges, gelatin or blister packs from which the powder may be administered with the aid of an inhalator or insufflator.

Depending upon the particular condition, or disease state, to be treated or prevented, additional therapeutic agents, which are normally administered to treat or prevent that condition, in other embodiments are administered together with the compounds of this disclosure.

The present disclosure is not to be limited in scope by the exemplified embodiments, which are intended as illustrations of single aspects of the disclosure. Such modifications are intended to fall within the scope of the disclosure. Moreover, any one or more features of any embodiment of the disclosure may be combined with any one or more other features of any other embodiment of the disclosure, without departing from the scope of the disclosure. For example, compounds described herein are equally applicable to the methods of treatment described herein.

### Dosing

In some embodiments, continuously or discontinuously dosages are administered, for example once, twice or more per cycle or course of treatment, which in other embodiments are repeated for example every 7, 14, 21 or 28 days.

In other embodiments, the compounds of the present disclosure are continuously or discontinuously administered to a subject systemically, for example, intravenously, orally, subcutaneously, intramuscular, intradermal, or parenterally. In other embodiments, the compounds of the present disclosure are continuously or discontinuously administered to a subject locally. Non-limiting examples of local delivery systems include the use of intraluminal medical devices that include intravascular drug delivery catheters, wires, pharmacological stents and endoluminal paving.

In other embodiments the compounds of the present disclosure are further continuously or discontinuously administered to a subject in combination with a targeting agent to achieve high local concentration of the compound at the target site. In some embodiments, the compounds of the present disclosure are formulated for fast-release or slow-release with the objective of maintaining the drugs or agents in contact with target tissues for a period ranging from hours to weeks.

In other embodiments the optimum method and order of continuously or discontinuously dosing or administration and the dosage amounts and regime are readily determined using conventional methods and in view of the information set out herein.

In various embodiments, the compounds disclosed herein are administered continuously or discontinuously.

In one embodiment, the compound is administered once or twice daily for 28 days with patients then being evaluated for continuation of treatment. In another embodiment, the compound is administered once or twice daily dosing on a 14 days on, 7 days off therapy schedule, cycling every 21 days. In various embodiments, the therapy can last up to 12 months. In some embodiments, the therapy lasts for at least two months, at least three months, at least four months, at least five months, at least six months, at least seven months, at least eight months, at least nine months, or at least eleven months.

In addition to the aforementioned examples and embodiments of dosages, cycles, and schedules of cycles, numerous permutations of the aforementioned dosages, cycles, and schedules of cycles for the co-administration of a compound with a second chemotherapeutic compound, radiotherapy, or surgery are contemplated herein and in some embodiments are administered according to the patient, type of cancer, and/or appropriate treatment schedule as determined by qualified medical professionals.

The compounds according to the disclosure are effective over a wide dosage range. For example, in the treatment of adult humans, dosages from about 0.01 to about 10,000 mg, from about 0.5 to about 1000 mg, from about 1 to about 500 mg per day, and from about 5 to about 100 mg per day are examples of dosages that in some embodiments are used. In some embodiments, the compound is administered in an amount of about 20 mg/day to about 5 g/day, or about 80 mg/day to about 1 g/day. In some embodiments, the compound is administered in an amount of about 5 to about 500 mg/day. In some embodiments, the compound is administered in an amount of about 5 to about 250 mg/day. In some embodiments, the compound is administered in an amount of about 20 to about 200 mg/day. In some embodiments, the compound is administered in an amount of about 20 to about 150 mg/day. In various embodiments, the compound is administered in an amount of about 20 mg/day, about 30 mg/day, about 40 mg/day, about 50 mg/day, about 60 mg/day, about 70 mg/day, about 80 mg/day, about 90 mg/day, about 100 mg/day, about 110 mg/day, about 120 mg/day, about 130 mg/day, about 140 mg/day, about 150 mg/day, about 160 mg/day, about 170 mg/day, about 180 mg/day, about 190 mg/day or about 200 mg/day or more.

In some embodiments, the compound is administered in a dosage of about 1 mg/kg/day to about 120 mg/kg/day, for example about 10 to about 100 mg/kg/day, in other embodiments in a dosage of about 60 mg/kg/day. In some embodiments, the compound is administered in a dosage of about 2 to about 10 mg/kg. In some embodiments the compound is administered in a dosage of about 5 mg/kg. In some embodiments the compound is administered in an amount of about 10 mg/kg. In some embodiments the compound is administered in an amount of about 20 mg/kg. In some embodiments the compound is administered in an amount of about 30 mg/kg. In some embodiments the compound is administered in an amount of about 40 mg/kg. In some embodiments the compound is administered in an amount of about 50 mg/kg. In some embodiments the compound is administered in an amount of about 60 mg/kg.

In various embodiments, the compounds administered once a day, twice a day, three times a day, or four times a day. In specific embodiments, the compounds are administered twice a day.

The exact dosage will depend upon the route of administration, the form in which the compound is administered, the subject to be treated, the body weight of the subject to be treated, and the preference and experience of the attending physician.

### Combination Therapy

In another aspect, the disclosure provides combination therapies for treating or inhibiting the onset of a cell proliferative disorder or inflammatory disorders. In one embodiment, are combination therapies for treating or inhibiting the onset of a cell proliferative disorder or a disorder to inflammation in a subject. The combination therapy comprises continuously or discontinuously dosing or administering to the subject a therapeutically or prophylactically effective amount of a compound of the formulas described herein, and one or more other anti-cell proliferation therapy including chemotherapy, radiation therapy, gene therapy and immunotherapy.

In another aspect, the compounds of the disclosure are continuously or discontinuously administered in combination with chemotherapy. As used herein, chemotherapy refers to a therapy involving a chemotherapeutic agent. In some embodiments, a variety of chemotherapeutic agents are used in the combined treatment methods disclosed herein. Chemotherapeutic agents contemplated as exemplary, include, but are not limited to: platinum compounds (e.g., cisplatin, carboplatin, oxaliplatin); taxane compounds (e.g., paclitaxcel, docetaxol); campotothecin compounds (irinotecan, topotecan); vinca alkaloids (e.g., vincristine, vinblastine, vinorelbine); anti-tumor nucleoside derivatives (e.g., 5-fluorouracil, leucovorin, gemcitabine, capecitabine)alkylating agents (e.g., cyclophosphamide, carmustine, lomustine, thiotepa); epipodophyllotoxins/podophyllotoxins (e.g. etoposide, teniposide); aromatase inhibitors (e.g., anastrozole, letrozole, exemestane); anti-estrogen compounds (e.g., tamoxifen, fulvestrant), antifolates (e.g., premetrexed disodium); hypomethylating agents (e.g., azacitidine); biologics (e.g., gemtuzamab, cetuximab, rituximab, pertuzumab, trastuzumab, bevacizumab, erlotinib); antibiotics/anthracylines (e.g. idarubicin, actinomycin D, bleomycin, daunorubicin, doxorubicin, mitomycin C, dactinomycin, carminomycin, daunomycin); antimetabolites (e.g., clofarabine, aminopterin, cytosine arabinoside, methotrexate); tubulin-binding agents (e.g. combretastatin, colchicine, nocodazole); topoisomerase inhibitors (e.g., camptothecin); differentiating agents (e.g., retinoids, vitamin D and retinoic acid); retinoic acid metabolism blocking agents (RAMBA) (e.g., accutane); kinase inhibitors (e.g., flavoperidol, imatinib mesylate, gefitinib, erlotinib, sunitinib, lapatinib, sorafinib, temsirolimus, dasatinib); farnesyltransferase inhibitors (e.g., tipifarnib); histone deacetylase inhibitors; inhibitors of the ubiquitin-proteasome pathway (e.g., bortezomib, Yondelis).

Further useful agents include verapamil, a calcium antagonist found to be useful in combination with antineoplastic agents to establish chemosensitivity in tumor cells resistant to accepted chemotherapeutic agents and to potentiate the efficacy of such compounds in drug-sensitive malignancies. See Simpson W G, The calcium channel blocker verapamil and cancer chemotherapy. Cell Calcium. December 1985;6(6):449-67. Additionally, yet to emerge chemotherapeutic agents are contemplated as being useful in combination with the compound of the present disclosure.

In further embodiments, specific, non-limiting examples of combination therapies include use of the compounds of the present disclosure with agents found in the following pharmacotherapeutic classifications as indicated below. These lists should not be construed to be closed, but should instead serve as illustrative examples common to the relevant therapeutic area at present. Moreover, in other embodiments, combination regimens include a variety of routes of administration and should include oral, intravenous, intraocular, subcutaneous, dermal, and inhaled topical.

In some embodiments, therapeutic agents include chemotherapeutic agents, but are not limited to, anticancer agents, alkylating agents, cytotoxic agents, antimetabolic agents, hormonal agents, plant-derived agents, and biologic agents.

Examples of anti-tumor substances, for example those selected from, mitotic inhibitors, for example vinblastine; alkylating agents, for example cis-platin, carboplatin and cyclophosphamide; anti-metabolites, for example 5-fluorouracil, cytosine arabinside and hydroxyurea, or, for example, one of the preferred anti-metabolites disclosed in European Patent Application No. 239362 such as N-(5-[N-(3,4-dihydro-2-methyl-4-oxoquinazolin-6-yhnethyl)-N-methylamino]-2-thenoyl)-L-glutamic acid; growth factor inhibitors; cell cycle inhibitors; intercalating antibiotics, for example adriamycin and bleomycin; enzymes, for example, interferon; and anti-hormones, for example anti-estrogens such as Nolvadex™ (tamoxifen) or, for example anti-androgens such as Casodex™ (4′-cyano-3-(4-fluorophenylsulphonyl)-2-hydroxy-2-methyl-3′-(trifluoromethyl)propionanilide). Such conjoint treatment may be achieved by way of the simultaneous, sequential or separate dosing of the individual components of treatment.

Alkylating agents are polyfunctional compounds that have the ability to substitute alkyl groups for hydrogen ions. Examples of alkylating agents include, but are not limited to, bischloroethylamines (nitrogen mustards, e.g. chlorambucil, cyclophosphamide, ifosfamide, mechlorethamine, melphalan, uracil mustard), aziridines (e.g. thiotepa), alkyl alkone sulfonates (e.g. busulfan), nitrosoureas (e.g. carmustine, lomustine, streptozocin), nonclassic alkylating agents (altretamine, dacarbazine, and procarbazine), platinum compounds (carboplastin and cisplatin). These compounds react with phosphate, amino, hydroxyl, sulfihydryl, carboxyl, and imidazole groups. Under physiological conditions, these drugs ionize and produce positively charged ion that attach to susceptible nucleic acids and proteins, leading to cell cycle arrest and/or cell death. In some embodiments, combination therapy including a compound of Formula (V) as described herein and an alkylating agent has therapeutic synergistic effects on cancer and reduces side effects associated with these chemotherapeutic agents.

Cytotoxic agents are a group of drugs that produced in a manner similar to antibiotics as a modification of natural products. Examples of cytotoxic agents include, but are not limited to, anthracyclines (e.g. doxorubicin, daunorubicin, epirubicin, idarubicin and anthracenedione), mitomycin C, bleomycin, dactinomycin, plicatomycin. These cytotoxic agents interfere with cell growth by targeting different cellular components. For example, anthracyclines are generally believed to interfere with the action of DNA topoisomerase II in the regions of transcriptionally active DNA, which leads to DNA strand scissions. Bleomycin is generally believed to chelate iron and forms an activated complex, which then binds to bases of DNA, causing strand scissions and cell death. In some embodiments, combination therapy including a compound of Formula (V) as described herein and a cytotoxic agent has therapeutic synergistic effects on cancer and reduces side effects associated with these chemotherapeutic agents.

Antimetabolic agents are a group of drugs that interfere with metabolic processes vital to the physiology and proliferation of cancer cells. Actively proliferating cancer cells require continuous synthesis of large quantities of nucleic acids, proteins, lipids, and other vital cellular constituents. Many of the antimetabolites inhibit the synthesis of purine or pyrimidine nucleosides or inhibit the enzymes of DNA replication. Some antimetabolites also interfere with the synthesis of ribonucleosides and RNA and/or amino acid metabolism and protein synthesis as well. By interfering with the synthesis of vital cellular constituents, antimetabolites can delay or arrest the growth of cancer cells. Examples of antimetabolic agents include, but are not limited to, fluorouracil (5-FU), floxuridine (5-FUdR), methotrexate, leucovorin, hydroxyurea, thioguanine (6-TG), mercaptopurine (6-MP), cytarabine, pentostatin, fludarabine phosphate, cladribine (2-CDA), asparaginase, and gemcitabine. In other embodiments, combination therapy including a compound of Formula (V) as described herein and an antimetabolic agent has therapeutic synergistic effects on cancer and reduces side effects associated with these chemotherapeutic agents.

Hormonal agents are a group of drug that regulate the growth and development of their target organs. Most of the hormonal agents are sex steroids and their derivatives and analogs thereof, such as estrogens, androgens, and progestins. These hormonal agents may serve as antagonists of receptors for the sex steroids to down regulate receptor expression and transcription of vital genes. Examples of such hormonal agents are synthetic estrogens (e.g. diethylstibestrol), antiestrogens (e.g. tamoxifen, toremifene, fluoxymesterol and raloxifene), antiandrogens (bicalutamide, nilutamide, flutamide), aromatase inhibitors (e.g., aminoglutethimide, anastrozole and tetrazole), ketoconazole, goserelin acetate, leuprolide, megestrol acetate and mifepristone. In other embodiments, combination therapy including a compound of Formula (V) as described herein and a hormonal agent has therapeutic synergistic effects on cancer and reduces side effects associated with these chemotherapeutic agents.

Plant-derived agents are a group of drugs that are derived from plants or modified based on the molecular structure of the agents. Examples of plant-derived agents include, but are not limited to, vinca alkaloids (e.g., vincristine, vinblastine, vindesine, vinzolidine and vinorelbine), podophyllotoxins (e.g., etoposide (VP-16) and teniposide (VM-26)), taxanes (e.g., paclitaxel and docetaxel). These plant-derived agents generally act as antimitotic agents that bind to tubulin and inhibit mitosis. Podophyllotoxins such as etoposide are believed to interfere with DNA synthesis by interacting with topoisomerase II, leading to DNA strand scission. In other embodiments, combination therapy including a compound of Formula (V) as described herein and a plant-derived agent having therapeutic synergistic effects on cancer and reducing side effects associated with these chemotherapeutic agents.

Biologic agents are a group of biomolecules that elicit cancer/tumor regression when used alone or in combination with chemotherapy and/or radiotherapy. Examples of biologic agents include, but are not limited to, immuno-modulating proteins such as cytokines, monoclonal antibodies against tumor antigens, tumor suppressor genes, and cancer vaccines. In another embodiment is a combination therapy a compound of Formula (V) as described herein and a biologic agent having therapeutic synergistic effects on cancer, enhance the patient's immune responses to tumorigenic signals, and reduce potential side effects associated with this chemotherapeutic agent.

In some embodiments, for the treatment of oncologic diseases, proliferative disorders, and cancers, compounds according to the present disclosure are administered with an agent selected from the group comprising: aromatase inhibitors, antiestrogen, anti-androgen, corticosteroids, gonadorelin agonists, topoisomerase 1 and 2 inhibitors, microtubule active agents, alkylating agents, nitrosoureas, antineoplastic antimetabolites, platinum containing compounds, lipid or protein kinase targeting agents, IMiDs, protein or lipid phosphatase targeting agents, antiangiogenic agents, Akt inhibitors, IGF-I inhibitors, FGF3 modulators, mTOR inhibitors, Smac mimetics, HDAC inhibitors, agents that induce cell differentiation, bradykinin 1 receptor antagonists, angiotensin II antagonists, cyclooxygenase inhibitors, heparanase inhibitors, lymphokine inhibitors, cytokine inhibitors, IKK inhibitors, P38MAPK inhibitors, HSP90 inhibitors, multlikinase inhibitors, bisphosphanates, rapamycin derivatives, anti-apoptotic pathway inhibitors, apoptotic pathway agonists, PPAR agonists, inhibitors of Ras isoforms, telomerase inhibitors, protease inhibitors, metalloproteinase inhibitors, aminopeptidase inhibitors, dacarbazine (DTIC), actinomycins C₂, C₃, D, and F₁, cyclophosphamide, melphalan, estramustine, maytansinol, rifamycin, streptovaricin, doxorubicin, daunorubicin, epirubicin, idarubicin, detorubicin, carminomycin, idarubicin, epirubicin, esorubicin, mitoxantrone, bleomycins A, A₂, and B, camptothecin, Irinotecan®, Topotecan®, 9-aminocamptothecin, 10,11-methylenedioxycamptothecin, 9-nitrocamptothecin, bortezomib, temozolomide, TAS103, NPI0052, combretastatin, combretastatin A-2, combretastatin A-4, calicheamicins, neocarcinostatins, epothilones A B, C, and semi-synthetic variants, Herceptin®, Rituxan®, CD40 antibodies, asparaginase, interleukins, interferons, leuprolide, and pegaspargase, 5-fluorouracil, fluorodeoxyuridine, ptorafur, 5′-deoxyfluorouridine, UFT, MITC, S-1 capecitabine, diethylstilbestrol, tamoxifen, toremefine, tolmudex, thymitaq, flutamide, fluoxymesterone, bicalutamide, finasteride, estradiol, trioxifene, dexamethasone, leuproelin acetate, estramustine, droloxifene, medroxyprogesterone, megesterol acetate, aminoglutethimide, testolactone, testosterone, diethylstilbestrol, hydroxyprogesterone, mitomycins A, B and C, porfiromycin, cisplatin, carboplatin, oxaliplatin, tetraplatin, platinum-DACH, ormaplatin, thalidomide, lenalidomide, CI-973, telomestatin, CHIR258, Rad 001, SAHA, Tubacin, 17-AAG, sorafenib, JM-216, podophyllotoxin, epipodophyllotoxin, etoposide, teniposide, Tarceva®, Iressa®, Imatinib®, Miltefosine®, Perifosine®, aminopterin, methotrexate, methopterin, dichloro-methotrexate, 6-mercaptopurine, thioguanine, azattuoprine, allopurinol, cladribine, fludarabine, pentostatin, 2-chloroadenosine, deoxycytidine, cytosine arabinoside, cytarabine, azacitidine, 5-azacytosine, gencitabine, 5-azacytosine-arabinoside, vincristine, vinblastine, vinorelbine, leurosine, leurosidine and vindesine, paclitaxel, taxotere and docetaxel.

Cytokines possess profound immunomodulatory activity. Some cytokines such as interleukin-2 (IL-2, aldesleukin) and interferon have demonstrated antitumor activity and have been approved for the treatment of patients with metastatic renal cell carcinoma and metastatic malignant melanoma. IL-2 is a T-cell growth factor that is central to T-cell-mediated immune responses. The selective antitumor effects of IL-2 on some patients are believed to be the result of a cell-mediated immune response that discriminate between self and nonself. In some embodiments, examples of interleukins that are used in conjunction with a compound of Formula (V) as described herein include, but are not limited to, interleukin 2 (IL-2), and interleukin 4 (IL-4), interleukin 12 (IL-1 2).

Interferons include more than 23 related subtypes with overlapping activities, all of the IFN subtypes within the scope of the present disclosure. IFN has demonstrated activity against many solid and hematologic malignancies, the later appearing to be particularly sensitive.

In yet other embodiments, other immuno-modulating agents are used in conjunction with the compounds presented herein to inhibit abnormal cell growth. Examples of such immuno-modulating agents include, but are not limited to bacillus Calmette-Guerin, levamisole, and octreotide, a long-acting octapeptide that mimics the effects of the naturally occurring hormone somatostatin.

Monoclonal antibodies against tumor antigens are antibodies elicited against antigens expressed by tumors, preferably tumor-specific antigens. For example, monoclonal antibody HERCEPTIN® (Trastruzumab) is raised against human epidermal growth factor receptor2 (HER2) that is overexpressed in some breast tumors including metastatic breast cancer. Overexpression of HER2 protein is associated with more aggressive disease and poorer prognosis in the clinic. HERCEPTIN® is used as a single agent for the treatment of patients with metastatic breast cancer whose tumors over express the HER2 protein. In some embodiments are combination therapy including a compound of Formula (V) as described herein and HERCEPTIN® having therapeutic synergistic effects on tumors, especially on metastatic cancers.

Another example of monoclonal antibodies against tumor antigens is RITUXAN® (Rituximab) that is raised against CD20 on lymphoma cells and selectively deplete normal and malignant CD20⁺pre-B and mature B cells. RITUXAN® is used as single agent for the treatment of patients with relapsed or refractory low-grade or follicular, CD20⁺, B cell non-Hodgkin's lymphoma. In another embodiment is a combination therapy including a compound of Formula (V) as described herein and RITUXAN® having therapeutic synergistic effects not only on lymphoma, but also on other forms or types of malignant tumors.

Tumor suppressor genes are genes that function to inhibit the cell growth and division cycles, thus preventing the development of neoplasia. Mutations in tumor suppressor genes cause the cell to ignore one or more of the components of the network of inhibitory signals, overcoming the cell cycle check points and resulting in a higher rate of controlled cell growth-cancer. Examples of the tumor suppressor genes include, but are not limited to, DPC-4, NF-1, NF-2, RB, p53, WT1, BRCA1 and BRCA2.

DPC-4 is involved in pancreatic cancer and participates in a cytoplasmic pathway that inhibits cell division. NF-1 codes for a protein that inhibits Ras, a cytoplasmic inhibitory protein. NF-1 is involved in neurofibroma and pheochromocytomas of the nervous system and myeloid leukemia. NF-2 encodes a nuclear protein that is involved in meningioma, schwanoma, and ependymoma of the nervous system. RB codes for the pRB protein, a nuclear protein that is a major inhibitor of cell cycle. RB is involved in retinoblastoma as well as bone, bladder, small cell lung and breast cancer. P53 codes for p53 protein that regulates cell division and can induce apoptosis. Mutation and/or inaction of p53 is found in a wide ranges of cancers. WT1 is involved in Wilms tumor of the kidneys. BRCA1 is involved in breast and ovarian cancer, and BRCA2 is involved in breast cancer. The tumor suppressor gene can be transferred into the tumor cells where it exerts its tumor suppressing functions. In another embodiment is a combination therapy including a compound of Formula (V) as described herein and a tumor suppressor having therapeutic synergistic effects on patients suffering from various forms of cancer.

Cancer vaccines are a group of agents that induce the body's specific immune response to tumors. Most of cancer vaccines under research and development and clinical trials are tumor-associated antigens (TAAs). TAA are structures (i.e. proteins, enzymes or carbohydrates) which are present on tumor cells and relatively absent or diminished on normal cells. By virtue of being fairly unique to the tumor cell, TAAs provide targets for the immune system to recognize and cause their destruction. Example of TAAs include, but are not limited to gangliosides (GM2), prostate specific antigen (PSA), alpha-fetoprotein (AFP), carcinoembryonic antigen (CEA) (produced by colon cancers and other adenocarcinomas, e.g. breast, lung, gastric, and pancreas cancers), melanoma associated antigens (MART-1, gp 100, MAGE 1,3 tyrosinase), papillomavirus E6 and E7 fragments, whole cells or portions/lysates of antologous tumor cells and allogeneic tumor cells.

In some embodiments, an additional component is used in the combination to augment the immune response to TAAs. Examples of adjuvants include, but are not limited to, bacillus Calmette-Guerin (BCG), endotoxin lipopolysaccharides, keyhole limpet hemocyanin (GKLH), interleukin-2 (IL-2), granulocyte-macrophage colony-stimulating factor (GM-CSF) and cytoxan, a chemotherapeutic agent which is believe to reduce tumor-induced suppression when given in low doses.

In another aspect, the disclosure provides compounds which are continuously or discontinuously administered in combination with radiation therapy. As used herein, “radiation therapy” refers to a therapy comprising exposing the subject in need thereof to radiation. Such therapy is known to those skilled in the art. In other embodiments, the appropriate scheme of radiation therapy is similar to those already employed in clinical therapies wherein the radiation therapy is used alone or in combination with other chemotherapeutics.

In another aspect, the disclosure provides compounds which are continuously or discontinuously administered in combination with a gene therapy. As used herein, “gene therapy” refers to a therapy targeting on particular genes involved in tumor development. Possible gene therapy strategies include the restoration of defective cancer-inhibitory genes, cell transduction or transfection with antisense DNA corresponding to genes coding for growth factors and their receptors, RNA-based strategies such as ribozymes, RNA decoys, antisense messenger RNAs and small interfering RNA (siRNA) molecules and the so-called ‘suicide genes’.

In other aspect, the disclosure provides compounds which are continuously or discontinuously administered in combination with an immunotherapy. As used herein, “immunotherapy” refers to a therapy targeting particular protein involved in tumor development via antibodies specific to such protein. For example, monoclonal antibodies against vascular endothelial growth factor have been used in treating cancers.

In other embodiments, where a second pharmaceutical is used in addition to a compound of the disclosure, the two pharmaceuticals are continuously or discontinuously administered simultaneously (e.g. in separate or unitary compositions) sequentially in either order, at approximately the same time, or on separate dosing schedules. In further embodiments, the two compounds are continuously or discontinuously administered within a period and in an amount and manner that is sufficient to ensure that an advantageous or synergistic effect is achieved. It will be appreciated that in some embodiments, the method and order of administration and the respective dosage amounts and regimes for each component of the combination will depend on the particular chemotherapeutic agent being administered in conjunction with the compound of the present disclosure, their route of administration, the particular tumor being treated and the particular host being treated.

In yet another embodiment, one or more compounds are administered with one or more therapeutic agents for the treatment or prevention of various diseases, including, for example, cancer and inflammation. In various embodiments, combination therapies comprising a microtubule stabilizing compound refers to (1) pharmaceutical compositions that comprise one or more compounds described herein, such as compounds of Formula (V) in combination with one or more therapeutic agents (e.g., one or more therapeutic agents described herein); and (2) co-administration of one or more compounds with one or more therapeutic agents wherein the compounds presented herein including for example, compounds of Formula (V) and therapeutic agent have not been formulated in the same compositions (but in some embodiments, are present within the same kit or package, such as a blister pack or other multi-chamber package; connected, separately sealed containers (e.g., foil pouches) that in further embodiments are separated by the user; or a kit where the compound(s) and other therapeutic agent(s) are in separate vessels). In further embodiments, when using separate formulations, the compound of Formula (V) is administered at the same, intermittent, staggered, prior to, subsequent to, or combinations thereof, with the administration of another therapeutic agent.

In certain embodiments, the compounds described herein, their pharmaceutically acceptable salts, prodrug, solvates, polymorphs, tautomers or isomers are administered in combination with another cancer therapy or therapies. In other embodiments, these additional cancer therapies are for example, surgery, and the methods described herein and combinations of any or all of these methods. In further embodiments, combination treatments occur sequentially or concurrently and the combination therapies are neoadjuvant therapies or adjuvant therapies.

In some embodiments, the compounds described herein are administered with an additional therapeutic agent. In these embodiments, the compounds described herein are in a fixed combination with the additional therapeutic agent or a non-fixed combination with the additional therapeutic agent.

By way of example only, if one of the side effects experienced by a patient upon receiving one of the compounds described herein is hypertension, then in some embodiments, it is appropriate to administer an anti-hypertensive agent in combination with the compound. Or, by way of example only, the therapeutic effectiveness of one of the compounds described herein is enhanced by administration of another therapeutic agent, the overall therapeutic benefit to the patient is enhanced. Or, by way of example only, in other embodiments, the benefit experienced by a patient is increased by administering one of the compounds described herein with another therapeutic agent (which also includes a therapeutic regimen) that also has therapeutic benefit. In any case, in some embodiments, regardless of the disease, disorder or condition being treated, the overall benefit experienced by the patient is simply additive of the two therapeutic agents or in further embodiments, the patient experiences a synergistic benefit.

In some embodiments, the appropriate doses of chemotherapeutic agents is generally similar to or less than those already employed in clinical therapies wherein the chemotherapeutics are administered alone or in combination with other chemotherapeutics.

By way of example only, platinum compounds are advantageously administered in a dosage of about 1 to about 500 mg per square meter (mg/m²) of body surface area, for example about 50 to about 400 mg/m², particularly for cisplatin in a dosage of about 75 mg/m² and for carboplatin in about 300 mg/m² per course of treatment. Cisplatin is not absorbed orally and must therefore be delivered via injection intravenously, subcutaneously, intratumorally or intraperitoneally.

By way of example only, taxane compounds are advantageously continuously or discontinuously administered in a dosage of about 50 to about 400 mg per square meter (mg/m²) of body surface area, for example about 75 to about 250 mg/m², particularly for paclitaxel in a dosage of about 175 to about 250 mg/m² and for docetaxel in about 75 to about 150 mg/m² per course of treatment.

By way of example only, camptothecin compounds are advantageously continuously or discontinuously administered in a dosage of about 0.1 to about 400 mg per square meter (mg/m²) of body surface area, for example about 1 to about 300 mg/m², particularly for irinotecan in a dosage of about 100 to about 350 mg/m² and for topotecan in about 1 to about 2 mg/m² per course of treatment.

By way of example only, in some embodiments, vinca alkaloids are advantageously continuously or discontinuously administered in a dosage of about 2 to about 30 mg per square meter (mg/m²) of body surface area, particularly for vinblastine in a dosage of about 3 to about 12 mg/m², for vincristine in a dosage of about 1 to about 2 mg/m², and for vinorelbine in dosage of about 10 to about 30 mg/m² per course of treatment.

By way of example only, in further embodiments, anti-tumor nucleoside derivatives are advantageously continuously or discontinuously administered in a dosage of about 200 to about 2500 mg per square meter (mg/m²) of body surface area, for example about 700 to about 1500 mg/m². 5-fluorouracil (5-FU) is commonly used via intravenous administration with doses ranging from about 200 to about 500 mg/m² (in some embodiments from about 3 to about 15 mg/kg/day). Gemcitabine is advantageously continuously or discontinuously administered in a dosage of about 800 to about 1200 mg/m² and capecitabine is advantageously continuously or discontinuously administered in about 1000 to about 2500 mg/m² per course of treatment.

By way of example only, in other embodiments, alkylating agents are advantageously continuously or discontinuously administered in a dosage of about 100 to about 500 mg per square meter (mg/m²) of body surface area, for example about 120 to about 200 mg/m², in other embodiments for cyclophosphamide in a dosage of about 100 to about 500 mg/m², for chlorambucil in a dosage of about 0.1 to about 0.2 mg/kg of body weight, for carmustine in a dosage of about 150 to about 200 mg/m², and for lomustine in a dosage of about 100 to about 150 mg/m² per course of treatment.

By way of example only, in yet other embodiments podophyllotoxin derivatives are advantageously continuously or discontinuously administered in a dosage of about 30 to about 300 mg per square meter (mg/m2) of body surface area, for example about 50 to about 250 mg/m², particularly for etoposide in a dosage of about 35 to about 100 mg/m² and for teniposide in about 50 to about 250 mg/m² per course of treatment.

By way of example only, in other embodiments, anthracycline derivatives are advantageously continuously or discontinuously administered in a dosage of about 10 to about 75 mg per square meter (mg/m²) of body surface area, for example about 15 to about 60 mg/m², particularly for doxorubicin in a dosage of about 40 to about 75 mg/m², for daunorubicin in a dosage of about 25 to about 45mg/m², and for idarubicin in a dosage of about 10 to about 15 mg/m² per course of treatment.

By way of example only, in further embodiments, anti-estrogen compounds are advantageously continuously or discontinuously administered in a dosage of about 1 to about 100 mg daily depending on the particular agent and the condition being treated. Tamoxifen is advantageously administered orally in a dosage of about 5 to about 50 mg, about 10 to about 20 mg twice a day, continuing the therapy for sufficient time to achieve and maintain a therapeutic effect. Toremifene is advantageously continuously or discontinuously administered orally in a dosage of about 60 mg once a day, continuing the therapy for sufficient time to achieve and maintain a therapeutic effect. Anastrozole is advantageously continuously or discontinuously administered orally in a dosage of about 1 mg once a day. Droloxifene is advantageously continuously or discontinuously administered orally in a dosage of about 20-100 mg once a day. Raloxifene is advantageously continuously or discontinuously administered orally in a dosage of about 60 mg once a day. Exemestane is advantageously continuously or discontinuously administered orally in a dosage of about 25 mg once a day.

By way of example only, in further embodiments, biologics are advantageously continuously or discontinuously administered in a dosage of about 1 to about 5 mg per square meter (mg/m²) of body surface area, or as known in the art, if different. For example, trastuzumab is advantageously administered in a dosage of 1 to about 5 mg/m², in other embodiments, from about 2 to about 4 mg/m² per course of treatment.

In other embodiments, when a compound is administered with an additional treatment such as radiotherapy, the radiotherapy is administered at 1 day, 2 days, 3 days, 4 days, 5 days, 6 days, 7 days, 14 days, 21 days, or 28 days after administration of at least one cycle of a compound. In some embodiments, the radiotherapy is administered at 1 day, 2 days, 3 days, 4 days, 5 days, 6 days, 7 days, 14 days, 21 days, or 28 days before administration of at least one cycle of a compound. In additional embodiments, the radiotherapy is administered in any variation of timing with any variation of the aforementioned cycles for a compound. In other embodiments, additional schedules for co-administration of radiotherapy with cycles of a compound are further determined by appropriate testing, clinical trials, or in some embodiments are determined by qualified medical professionals.

When a compound is administered with an additional treatment such as surgery, the compound is administered 1, 2, 3, 4, 5, 6, 7, 14, 21, or 28 days prior to surgery. In additional embodiments, at least one cycle of the compound is administered 1, 2, 3, 4, 5, 6, 7, 14, 21, or 28 days after surgery. In yet further embodiments, additional variations of administering compound cycles in anticipation of surgery, or after the occurrence of surgery, are further determined by appropriate testing and/or clinical trials, or in some embodiments are determined by assessment of qualified medical professionals.

Other therapies include, but are not limited to administration of other therapeutic agents, radiation therapy or both. In the instances where the compounds described herein are administered with other therapeutic agents, the compounds described herein need not be administered in the same pharmaceutical composition as other therapeutic agents, and may, because of different physical and chemical characteristics, be administered by a different route. For example, in some embodiments, the compounds/compositions are administered orally to generate and maintain good blood levels thereof, while the other therapeutic agent is administered intravenously. In some embodiments, the initial administration is made according to established protocols, and then, based upon the observed effects, the dosage, modes of administration and times of administration in other embodiments, is modified by the skilled clinician. The particular choice of compound (and where appropriate, other therapeutic agent and/or radiation) will depend upon the diagnosis of the attending physicians and their judgment of the condition of the patient and the appropriate treatment protocol.

In other embodiments, the compounds and compositions described herein (and where appropriate chemotherapeutic agent and/or radiation) is administered concurrently (e.g., simultaneously, essentially simultaneously or within the same treatment protocol) or sequentially, depending upon the nature of the disease, the condition of the patient, and the actual choice of chemotherapeutic agent and/or radiation to be administered in conjunction (i.e., within a single treatment protocol) with the compound/composition.

In combinational applications and uses, the compound/composition and the chemotherapeutic agent and/or radiation need not be administered simultaneously or essentially simultaneously, and the initial order of administration of the compound/composition, and in other embodiments, the chemotherapeutic agent and/or radiation, is not important. Thus, in some embodiments, the compounds/compositions of the present disclosure are administered first followed by the administration of the chemotherapeutic agent and/or radiation; or the chemotherapeutic agent and/or radiation is administered first followed by the administration of the compounds/compositions described herein. In further embodiments, this alternate administration is repeated during a single treatment protocol. With the teachings described herein, the determination of the order of administration, and the number of repetitions of administration of each therapeutic agent during a treatment protocol, would be within the knowledge of the skilled physician after evaluation of the disease being treated and the condition of the patient. For example, in some embodiments, the chemotherapeutic agent and/or radiation is administered first, especially if it is a cytotoxic agent, and then the treatment continued with the administration of the compounds/compositions of the present disclosure followed, where determined advantageous, by the administration of the chemotherapeutic agent and/or radiation, and so on until the treatment protocol is complete. Thus, in other embodiments and in accordance with experience and knowledge, the practicing physician modifies each protocol for the administration of the compound/composition for treatment according to the individual patient's needs, as the treatment proceeds. The attending clinician, in judging whether treatment is effective at the dosage administered, will consider the general well-being of the patient as well as more definite signs such as relief of disease-related symptoms, inhibition of tumor growth, actual shrinkage of the tumor, or inhibition of metastasis. Size of the tumor can be measured by standard methods such as radiological studies, e.g., CAT or MRI scan, and successive measurements can be used to judge whether or not growth of the tumor has been retarded or even reversed. In further embodiments, relief of disease-related symptoms such as pain, and improvement in overall condition is used to help judge effectiveness of treatment.

In some embodiments, a composition described herein is administered before the administration of one or more chemotherapeutic agents. As non-limiting examples of this embodiment, the chemotherapeutic agent is administered hours (e.g. one, five, ten, etc.) or days (e.g., one, two, three, etc.) after administration of the composition described herein. In some embodiments, the subsequent administration is shortly after (e.g., within an hour) administration of the compound described herein.

Anti-emetic agents are a group of drugs effective for treatment of nausea and emesis (vomiting). Cancer therapies frequently cause urges to vomit and/or nausea. Many anti-emetic drugs target the 5-HT₃ seratonin receptor which is involved in transmitting signals for emesis sensations. These 5-HT₃ antagonists include, but are not limited to, dolasetron (Anzemet®), granisetron (Kytril®), ondansetron (Zofran®), palonosetron and tropisetron. Other anti-emetic agents include, but are not limited to, the dopamine receptor antagonists such as chlorpromazine, domperidone, droperidol, haloperidol, metaclopramide, promethazine, and prochlorperazine; antihistamines such as cyclizine, diphenhydramine, dimenhydrinate, meclizine, promethazine, and hydroxyzine; lorazepram, scopolamine, dexamethasone, emetrol®, propofol, and trimethobenzamide. Administration of these anti-emetic agents in addition to the above described combination treatment will manage the potential nausea and emesis side effects caused by the combination treatment.

Immuno-restorative agents are a group of drugs that counter the immuno-suppressive effects of many cancer therapies. The therapies often cause myelosuppression, a substantial decrease in the production of leukocytes (white blood cells). The decreases subject the patient to a higher risk of infections. Neutropenia is a condition where the concentration of neutrophils, the major leukocyte, is severely depressed. Immuno-restorative agents are synthetic analogs of the hormone, granulocyte colony stimulating factor (G-CSF), and act by stimulating neutrophil production in the bone marrow. These include, but are not limited to, filgrastim (Neupogen®), PEG-filgrastim (Neulasta®) and lenograstim. Administration of these immuno-restorative agents in addition to the above described combination treatment will manage the potential myelosuppression effects caused by the combination treatment.

Antibiotic agents are a group of drugs that have anti-bacterial, anti-fungal, and anti-parasite properties. Antibiotics inhibit growth or causes death of the infectious microorganisms by various mechanisms such as inhibiting cell wall production, preventing DNA replication, or deterring cell proliferation. Potentially lethal infections occur from the myelosuppression side effects due to cancer therapies. The infections can lead to sepsis where fever, widespread inflammation, and organ dysfunction arise. Antibiotics manage and abolish infection and sepsis and include, but are not limited to, amikacin, gentamicin, kanamycin, neomycin, netilmicin, streptomycin, tobramycin, loracarbef, ertapenem, cilastatin, meropenem, cefadroxil, cefazolin, cephalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, teicoplanin, vancomycin, azithromycin, clarithromycin, dirithromycin, erthromycin, roxithromycin, troleandomycin, aztreonam, amoxicillin, ampicillin, azlocillin, carbenicillin, cloxacillin, dicloxacillin, flucloxacillin, mezlocillin, nafcillin, penicillin, piperacillin, ticarcillin, bacitracin, colistin, polymyxin B, ciprofloxacin, enoxacin, gatifloxacin, levofloxacin, lomefloxacin, moxifloxacin, norfloxacin, ofloxacin, trovafloxacin, benzolamide, bumetanide, chlorthalidone, clopamide, dichlorphenamide, ethoxzolamide, indapamide, mafenide, mefruside, metolazone, probenecid, sulfanilamides, sulfamethoxazole, sulfasalazine, sumatriptan, xipamide, democlocycline, doxycycline, minocycline, oxytetracycline, tetracycline, chloramphenical, clindamycin, ethambutol, fosfomycin, fusidic acid, furazolidone, isoniazid, linezolid, metronidazole, mupirocin, nitrofurantoin, platesimycin, pyrazinamide, dalfopristin, rifampin, spectinomycin, and telithromycin. Administration of these antibiotic agents in addition to the above described combination treatment will manage the potential infection and sepsis side effects caused by the combination treatment.

Anemia treatment agents are compounds directed toward treatment of low red blood cell and platelet production. In addition to myelosuppression, many cancer therapies also cause anemias, deficiencies in concentrations and production of red blood cells and related factors. Anemia treatment agents are recombinant analogs of the glycoprotein, erythropoeitin, and function to stimulate erythropoesis, the formation of red blood cells. Anemia treatment agents include, but are not limited to, recombinant erythropoietin (EPOGEN®, Dynopro®) and Darbepoetin alfa (Aranesp®). Administration of these anemia treatment agents in addition to the above described combination treatment will manage the potential anemia side effects caused by the combination treatment.

In some embodiments, pain and inflammation side effects arising from the described herein combination treatment are treated with compounds selected from the group comprising: corticosteroids, non-steroidal anti-inflammatories, muscle relaxants and combinations thereof with other agents, anesthetics and combinations thereof with other agents, expectorants and combinations thereof with other agents, antidepressants, anticonvulsants and combinations thereof; antihypertensives, opioids, topical cannabinoids, and other agents, such as capsaicin.

In some embodiments, for the treatment of pain and inflammation side effects, compounds according to the present disclosure are administered with an agent selected from the group comprising: betamethasone dipropionate (augmented and nonaugmented), betamethasone valerate, clobetasol propionate, prednisone, methyl prednisolone, diflorasone diacetate, halobetasol propionate, amcinonide, dexamethasone, dexosimethasone, fluocinolone acetononide, fluocinonide, halocinonide, clocortalone pivalate, dexosimetasone, flurandrenalide, salicylates, ibuprofen, ketoprofen, etodolac, diclofenac, meclofenamate sodium, naproxen, piroxicam, celecoxib, cyclobenzaprine, baclofen, cyclobenzaprine/lidocaine, baclofen/cyclobenzaprine, cyclobenzaprine/lidocaine/ketoprofen, lidocaine, lidocaine/deoxy-D-glucose, prilocaine, EMLA Cream (Eutectic Mixture of Local Anesthetics (lidocaine 2.5% and prilocaine 2.5%), guaifenesin, guaifenesin/ketoprofen/cyclobenzaprine, amitryptiline, doxepin, desipramine, imipramine, amoxapine, clomipramine, nortriptyline, protriptyline, duloxetine, mirtazepine, nisoxetine, maprotiline, reboxetine, fluoxetine, fluvoxamine, carbamazepine, felbamate, lamotrigine, topiramate, tiagabine, oxcarbazepine, carbamezipine, zonisamide, mexiletine, gabapentin/clonidine, gabapentin/carbamazepine, carbamazepine/cyclobenzaprine, antihypertensives including clonidine, codeine, loperamide, tramadol, morphine, fentanyl, oxycodone, hydrocodone, levorphanol, butorphanol, menthol, oil of wintergreen, camphor, eucalyptus oil, turpentine oil; CB1/CB2 ligands, acetaminophen, infliximab) nitric oxide synthase inhibitors, particularly inhibitors of inducible nitric oxide synthase; and other agents, such as capsaicin. Administration of these pain and inflammation analgesic agents in addition to the above described combination treatment will manage the potential pain and inflammation side effects caused by the combination treatment.

### Kits/Articles of Manufacture

For use in the therapeutic applications described herein, kits and articles of manufacture are also described herein. In some embodiments, kits comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In other embodiments, the containers are formed from a variety of materials such as glass or plastic.

The articles of manufacture provided herein contain packaging materials. Packaging materials for use in packaging pharmaceutical products include, but are not limited to, blister packs, bottles, tubes, inhalers, pumps, bags, vials, containers, syringes, bottles, and any packaging material suitable for a selected formulation and intended mode of administration and treatment.

For example, in one embodiment, the container(s) comprise one or more compounds described herein, optionally in a composition or in combination with another agent as disclosed herein. The container(s) optionally have a sterile access port (for example, in another embodiment, the container is an intravenous solution bag or a vial having a stopper pierceable by a hypodermic injection needle). Such kits optionally comprising a compound with an identifying description or label or instructions relating to its use in the methods described herein.

In another embodiment is a kit comprised of one or more additional containers, each with one or more of various materials (such as reagents, optionally in concentrated form, and/or devices) desirable from a commercial and user standpoint for use of a compound described herein. Non-limiting examples of such materials include, but not limited to, buffers, diluents, filters, needles, syringes; carrier, package, container, vial and/or tube labels listing contents and/or instructions for use, and package inserts with instructions for use. In a further embodiment, a set of instructions is included.

In yet another embodiment, a label is on or associated with the container. In a further embodiment a label is on a container when letters, numbers or other characters forming the label are attached, molded or etched into the container itself. In one embodiment, a label is associated with a container when it is present within a receptacle or carrier that also holds the container, e.g., as a package insert. In another embodiment, a label is used to indicate that the contents are to be used for a specific therapeutic application. In yet another embodiment, the label also indicates directions for use of the contents, such as in the methods described herein.

In certain embodiments, the pharmaceutical compositions are presented in a pack or dispenser device which contains one or more unit dosage forms containing a compound provided herein. In a further embodiment, the pack for example contains metal or plastic foil, such as a blister pack. In yet a further embodiment, the pack or dispenser device optionally is accompanied by instructions for administration. In another embodiment, the pack or dispenser is accompanied with a notice associated with the container in form prescribed by a governmental agency regulating the manufacture, use, or sale of pharmaceuticals, which notice is reflective of approval by the agency of the form of the drug for human or veterinary administration. Such notice, for example, in one embodiment, the labeling approved by the U.S. Food and Drug Administration for prescription drugs, or the approved product insert. Compositions containing a compound provided herein formulated in a compatible pharmaceutical carrier are also contemplated, placed in an appropriate container, and labeled for treatment of an indicated condition.

### Synthetic Procedures

In another aspect, methods for synthesizing the compounds described herein are provided. In some embodiments, the compounds described herein are prepared by the methods described below. The procedures and examples below are intended to illustrate those methods. Neither the procedures nor the examples should be construed as limiting the scope of the present disclosure in any way. In some embodiments, compounds described herein are also synthesized using standard synthetic techniques in combination with methods described herein. In further embodiments, solvents, temperatures and other reaction conditions presented herein vary according to the practice.

The starting materials used for the synthesis of the compounds as described herein can be obtained from commercial sources, such as Aldrich Chemical Co. (Milwaukee, Wis.), Sigma Chemical Co. (St. Louis, Mo.), or the starting materials can be synthesized. The compounds described herein, and other related compounds having different substituents can be synthesized using techniques and materials known to those of skill in the art, such as described, for example, in March, ADVANCED ORGANIC CHEMISTRY 4^{th} Ed., (Wiley 1992); Carey and Sundberg, ADVANCED ORGANIC CHEMISTRY 4^{th} Ed., Vols. A and B (Plenum 2000, 2001), and Green and Wuts, PROTECTIVE GROUPS IN ORGANIC SYNTHESIS 3^{rd} Ed., (Wiley 1999) (all of which are incorporated by reference in their entirety). General methods for the preparation of compounds as disclosed herein may be derived from known reactions in the field, and the reactions may be modified by the use of appropriate reagents and conditions, as would be recognized by the skilled person, for the introduction of the various moieties found in the formulae as provided herein. As a guide the following synthetic methods may be utilized.

Formation of Covalent Linkages by Reaction of an Electrophile with a Nucleophile

In some embodiments, the compounds described herein are modified using various electrophiles or nucleophiles to form new functional groups or substituents. The table below entitled “Examples of Covalent Linkages and Precursors Thereof” lists selected examples of covalent linkages and precursor functional groups which yield and can be used as guidance toward the variety of electrophiles and nucleophiles combinations available. Precursor functional groups are shown as electrophilic groups and nucleophilic groups.

Examples of Covalent Linkages and Precursors Thereof

| Covalent Linkage Product | Electrophile | Nucleophile |
|---|---|---|
| Carboxamides | Activated esters | Amines/anilines |
| Carboxamides | Acyl azides | Amines/anilines |
| Carboxamides | Acyl halides | Amines/anilines |
| Esters | Acyl halides | Alcohols/phenols |
| Esters | Acyl nitriles | Alcohols/phenols |
| Carboxamides | Acyl nitriles | Amines/anilines |
| Imines | Aldehydes | Amines/anilines |
| Hydrazones | Aldehydes or ketones | Hydrazines |
| Oximes | Aldehydes or ketones | Hydroxylamines |
| Alkyl amines | Alkyl halides | Amines/anilines |
| Esters | Alkyl halides | Carboxylic acids |
| Thioethers | Alkyl halides | Thiols |
| Ethers | Alkyl halides | Alcohols/phenols |
| Thioethers | Alkyl sulfonates | Thiols |
| Esters | Alkyl sulfonates | Carboxylic acids |
| Ethers | Alkyl sulfonates | Alcohols/phenols |
| Esters | Anhydrides | Alcohols/phenols |
| Carboxamides | Anhydrides | Amines/anilines |
| Thiophenols | Aryl halides | Thiols |
| Aryl amines | Aryl halides | Amines |
| Thioethers | Aziridines | Thiols |
| Boronate esters | Boronates | Glycols |
| Carboxamides | Carboxylic acids | Amines/anilines |
| Esters | Carboxylic acids | Alcohols |
| Hydrazines | Hydrazides | Carboxylic acids |
| N-acylureas or Anhydrides | Carbodiimides | Carboxylic acids |
| Esters | Diazoalkanes | Carboxylic acids |
| Thioethers | Epoxides | Thiols |
| Thioethers | Haloacetamides | Thiols |
| Ammotriazines | Halotriazines | Amines/anilines |
| Triazinyl ethers | Halotriazines | Alcohols/phenols |
| Amidines | Imido esters | Amines/anilines |
| Ureas | Isocyanates | Amines/anilines |
| Urethanes | Isocyanates | Alcohols/phenols |
| Thioureas | Isothiocyanates | Amines/anilines |
| Thioethers | Maleimides | Thiols |
| Phosphite esters | Phosphoramidites | Alcohols |
| Silyl ethers | Silyl halides | Alcohols |
| Alkyl amines | Sulfonate esters | Amines/anilines |
| Thioethers | Sulfonate esters | Thiols |
| Esters | Sulfonate esters | Carboxylic acids |
| Ethers | Sulfonate esters | Alcohols |
| Sulfonamides | Sulfonyl halides | Amines/anilines |
| Sulfonate esters | Sulfonyl halides | Phenols/alcohols |

### Protecting Groups

In the reactions described, it may be necessary to protect reactive functional groups, for example hydroxy, amino, imino, thio or carboxy groups, where these are desired in the final product, to avoid their unwanted participation in the reactions. Protecting groups are used to block some or all reactive moieties and prevent such groups from participating in chemical reactions until the protective group is removed. Protected derivatives are useful in the preparation of the compounds described herein or in themselves may be active as inhibitors. It is preferred that each protective group be removable by a different means. Protective groups that are cleaved under totally disparate reaction conditions fulfill the requirement of differential removal. Protective groups can be removed by acid, base, and hydrogenolysis. Groups such as trityl, dimethoxytrityl, acetal and t-butyldimethylsilyl are acid labile and may be used to protect carboxy and hydroxy reactive moieties in the presence of amino groups protected with Cbz groups, which are removable by hydrogenolysis, and Fmoc groups, which are base labile. Carboxylic acid and hydroxy reactive moieties may be blocked with base labile groups such as, but not limited to, methyl, ethyl, and acetyl in the presence of amines blocked with acid labile groups such as t-butyl carbamate or with carbamates that are both acid and base stable but hydrolytically removable.

Such protecting groups are described, for example, in the reference text of Greene and Wuts (Protective Groups in Organic Synthesis, 3^{rd} Edition, Wiley, 1999). By employing appropriate manipulation and protection of any chemical functionalities, synthesis of compounds to be employed in the processes not specifically set forth herein can be accomplished by methods analogous to the schemes set forth below.

Carboxylic acid and hydroxy reactive moieties may also be blocked with hydrolytically removable protective groups such as the benzyl group, while amine groups capable of hydrogen bonding with acids may be blocked with base labile groups such as Fmoc. Carboxylic acid reactive moieties may be protected by conversion to simple ester compounds as exemplified herein, or they may be blocked with oxidatively-removable protective groups such as 2,4-dimethoxybenzyl, while co-existing amino groups may be blocked with fluoride labile silyl carbamates.

Allyl blocking groups are useful in then presence of acid- and base-protecting groups since the former are stable and can be subsequently removed by metal or pi-acid catalysts. For example, an allyl-blocked carboxylic acid can be deprotected with a Pd-catalyzed reaction in the presence of acid labile t-butyl carbamate or base-labile acetate amine protecting groups. Yet another form of protecting group is a resin to which the compound or intermediate may be attached. As long as the residue is attached to the resin, that functional group is blocked and cannot react. Once released from the resin, the functional group is available to react.

Protecting or blocking groups may be selected from:

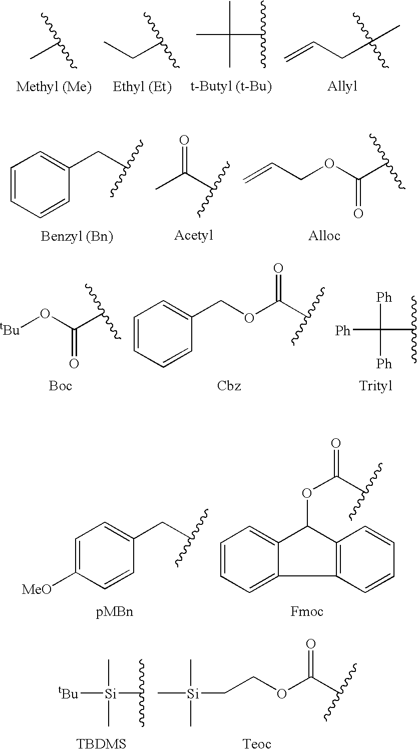

Other protecting groups, plus a detailed description of techniques applicable to the creation or protecting group and their removal are described in Greene and Wuts, Protective Groups in Organic Synthesis, 3rd Ed., John Wiley & Sons, New York, N.Y., 1999, and Kocienski, Protective Groups, Thieme Verlag, New York, N.Y., 1994, which are incorporated herein by reference in their entirety.

### Olefin Cross Metathesis

Olefin metathesis or transalkylidenation consists of an alkene double bond cleavage, followed by a statistical redistribution of alkylidene fragments. Self-dimerization reactions of the more valuable alkene may be minimized by the use of excess of the more readily available alkene. Olefin cross metathesis is a powerful and convenient synthetic technique in organic chemistry; however, as a general synthetic method, it has been limited by the lack of predictability in product selectivity and stereoselectivity. The olefin metathesis reaction was reported as early as 1955 in a Ti(II)-catalyzed polymerication of norbornene. 15 years later, Chauvin first proposed that olefin metathesis proceeds via metallacyclobutanes. Herisson, P. J.-L.; Chauvin, Y. Makrolmol. Chem. 1970, 141, 161-76. It is now generally accepted that both cyclic and acyclic olefin metathesis reactions proceed via metallacyclobutane and metal-carbene intermediates.

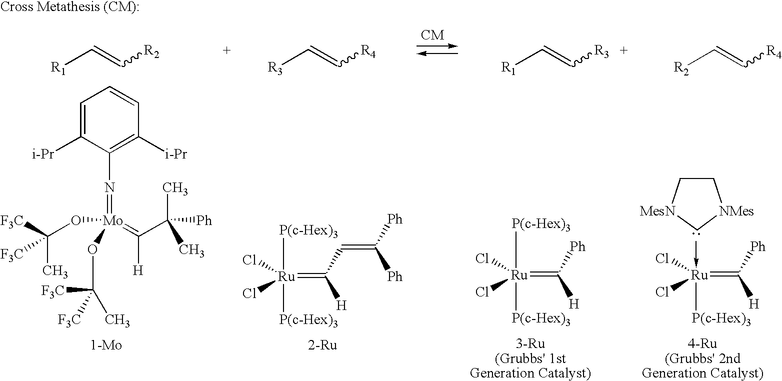

Grubb's Ru-based catalysts exhibit high reactivity in a variety of cross metathesis processes and show good tolerance toward many different organic functional groups.

The electron-rich tricyclohexyl phosphine ligands of the d⁶ Ru(II) metal center in alkylidenes of the Grubb's 1^{st} generation catalyst leads to increased metathesis activity. Grubb's 1^{st} generation catalyst is synthesized from RuCl₂(PPh₃), phenyldiazomethane, and tricyclohexylphosphine in a one-pot synthesis. The NHC ligand on Grubb's 2^{nd} generation catalyst stabilizes a 14 e⁻Ru intermediate in the catalytic cycle, making this catalyst more effective than 2-Ru and Grubb's 1^{st} generation catalyst shown above. The Grubb's 2^{nd} generation catalyst is synthesized from the combination of the 1^{st} generation catalyst and alkoxy-protected 1,3-dimesityl-4,5-dihydroimidazol-2-ylidene.

Schrock's alkoxy imidomolybdenum complex, 1-Mo is highly reactive toward a broad range of substrates. Titanium and tungsten-based catalysts have also been developed for cross metathesis.

In one embodiment, is a compound of Formula (I) produced by a process using a cross-metathesis catalyst. In another embodiment, the cross-metathesis catalyst is selected from a catalyst containing Ni, W, Ru, or Mo metal. In yet another embodiment, the cross-metathesis catalyst contains Ru. In some embodiments, the cross-metathesis catalyst is a 1^{st} generation Grubb's catalyst. In other embodiments, the cross-metathesis catalyst is a 2^{nd} generation Grubb's catalyst. In one embodiment, the cross-metathesis catalyst contains Mo metal. In another embodiment, the cross-metathesis catalyst contains Ru metal. In another embodiment, the cross-metathesis catalyst is selected from: dichloro(phenylmethylene)bis(tricyclohexylphosphine)ruthenium(II), [1,3-bis-(2,4,6-trimethylphenyl)-2-imidazolidinylidene]dichloro(phenylmethylene)(tricyclohexylphosphine)ruthenium(II), dichloro[[2-(1-methylethoxy)phenyl]methylene](tricyclohexylphosphine)ruthenium(II), [1,3-bis(2,4,6-trimethylphenyl)-2-imidazolidinylidene]dichloro[[2-(1-methylethoxy)phenyl]methylene]ruthenium(II), [1,3-bis(2-methylphenyl)-2-imidazolidinylidene]dichloro[[2-(1-methylethoxy)phenyl]methylene]ruthenium(II), [1,3-bis(2,4,6-trimethylphenyl)-2-imidazolidinylidene]dichloro[3-(2-pyridinyl-κN)propylidene-κC]ruthenium (II), [1,3-bis(2-methylphenyl)-2-imidazolidinylidene]dichloro(benzylidene)(tricyclohexylphosphine)ruthenium (II), dichloro(3-methyl-2-butenylidene)bis(tricyclohexylphosphine)ruthenium(II), [1,3-bis(2,4,6-trimethylphenyl)-2-imidazolidinylidene]dichloro(3-methyl-2-butenylidene)(tricyclohexylphosphine)ruthenium(II) [1,3-bis(2,4,6-trimethylphenyl)-2-imidazolidinylidene]dichloro(benzylidene)bis(3-bromopyridine)ruthenium(II).

### Further Forms of the Compounds

### Isomers

In some embodiments, the compounds described herein exist as geometric isomers. In other embodiments, the compounds described herein possess one or more double bonds. The compounds presented herein include all cis, trans, syn, anti, entgegen (E), and zusammen (Z) isomers as well as the corresponding mixtures thereof. In some situations, compounds exist as tautomers. The compounds described herein include all possible tautomers within the formulas described herein.

In other embodiments, the compounds described herein possess one or more stereocenters and in some embodiments, each center exist in the R or S configuration. The compounds described herein include all diastereomeric, enantiomeric, and epimeric forms as well as the corresponding mixtures thereof. In additional embodiments of the compounds and methods provided herein, mixtures of enantiomers and/or diastereoisomers, resulting from a single preparative step, combination, or interconversion are useful for the applications described herein.

In some embodiments, the compounds described herein are prepared as their individual stereoisomers by reacting a racemic mixture of the compound with an optically active resolving agent to form a pair of diastereoisomeric compounds or complexes, separating the diastereomers and recovering the optically pure enantiomers. While resolution of enantiomers can be carried out using covalent diastereomeric derivatives of the compounds described herein, dissociable complexes are preferred (e.g., crystalline diastereomeric salts). Diastereomers have distinct physical properties (e.g., melting points, boiling points, solubilities, reactivity, etc.) and in other embodiments, are readily separated by taking advantage of these dissimilarities. In further embodiments, the diastereomers are separated by chromatography, or in some embodiments, by separation/resolution techniques based upon differences in solubility. The single enantiomer of high optical purity (ee>90%) is then recovered, along with the resolving agent, by any practical means that would not result in racemization. A more detailed description of the techniques applicable to the resolution of stereoisomers of compounds from their racemic mixture can be found in Jean Jacques, Andre Collet, Samuel H. Wilen, “Enantiomers, Racemates and Resolutions,” John Wiley And Sons, Inc., 1981, herein incorporated by reference to the extent necessary.

### Labeled Compounds

It should be understood that the compounds described herein include their isotopically-labeled equivalents, including their use for treating disorders. For example, the disclosure provides for methods of treating diseases, by administering isotopically-labeled compounds of Formula (V) The isotopically-labeled compounds described herein can be administered as pharmaceutical compositions. Thus, the compounds described herein also include their isotopically-labeled isomers, which are identical to those recited herein, but for the fact that one or more atoms are replaced by an atom having an atomic mass or mass number different from the atomic mass or mass number usually found in nature. In other embodiments, examples of isotopes that are incorporated into compounds of the present disclosure include isotopes of hydrogen, carbon, nitrogen, oxygen, phosphorous, sulfur, fluorine and chloride, such as ²H, ³H, ¹¹C, ¹³C, ¹⁴C, ¹⁵N, ¹⁸0, ¹⁷O, ³¹P, ³²P, ³⁵S, ¹⁸F, and ³⁶Cl, respectively. Compounds described herein, pharmaceutically acceptable salts, esters, prodrugs, solvate, hydrates or derivatives thereof which contain the aforementioned isotopes and/or other isotopes of other atoms are within the scope of the disclosure. Certain isotopically-labeled compounds, for example those into which radioactive isotopes such as ³H and ¹⁴C are incorporated, are useful in drug and/or substrate tissue distribution assays. Tritiated, i. e., ³H and carbon-14, i.e., ¹⁴C, isotopes are, in some embodiments, used for their ease of preparation and detectability. Further, substitution with heavier isotopes such as deuterium, i. e., ²H, can afford certain therapeutic advantages resulting from greater metabolic stability, for example increased in vivo half-life or reduced dosage requirements and, hence, in other embodiments, is employed in some circumstances. In further embodiments, isotopically labeled compounds, pharmaceutically acceptable salts, esters, prodrugs, solvates, hydrates or derivatives thereof are generally prepared by carrying out procedures described herein, by substituting a readily available isotopically labeled reagent for a non-isotopically labeled reagent.

In further embodiments, the compounds described herein are labeled by other means, including, but not limited to, the use of chromophores or fluorescent moieties, bioluminescent labels, or chemiluminescent labels.

The present disclosure is not to be limited in scope by the embodiments disclosed herein, which are intended as illustrations of single aspects of the present disclosure. Indeed, various modifications of the aspects described herein are also contemplated. Such modifications are intended to fall within the scope of the present disclosure. Moreover, in some other embodiments, any one or more features of any embodiment is combined with any one or more other features of any other embodiment disclosed herein, without departing from the scope described herein.

The examples and preparations provided below further illustrate and exemplify the compounds of the present disclosure and methods of preparing such compounds. It is to be understood that the scope of the present disclosure is not limited in any way by the scope of the following examples and preparations.

### EXAMPLES

The present disclosure is further illustrated by the following examples, which should not be construed as limiting in any way. The experimental procedures to generate the data shown are discussed in more detail below. The disclosure has been described in an illustrative manner, and it is to be understood that the terminology used is intended to be in the nature of description rather than of limitation.

### I Chemical Syntheses

It should be understood that the following are provided for exemplary purposes and additional compounds and compounds with additional substitutions are contemplated by the present disclosure. For example, where a substituent is indicated in the para position of a ring, it should be understood that the substituent may be in the ortho or meta positions instead or that there may be an additional substituent in the ortho or meta positions. Also, where a substituent is exemplified on one compound, it should be understood that that substituent could also be attached to any of the other compounds described herein.

The detailed synthesis of various intermediates and precursors described herein can be found in Wender et al., J. Am. Chem. Soc. 2002, 124, 4956-7 and Wender et al., Org. Lett. 2006, 8, 1507-10 and references cited therein, and are incorporated herein by reference. Additional details regarding the synthesis of various intermediates and precursors described herein can be found in Ghosh et al., J. Am. Chem. Soc. 2000, 122, 11027-8; Ghosh et al., Tetrahedron Lett. 2001, 42, 3399-3401; Mulzer et al., Angew. Chem. Int. Ed. 2001, 40, 3842-46; Enev et al., J. Am. Chem. Soc. 2001, 123, 10764-65; Paterson et al., Org. Lett. 2001, 3, 3149-52; Paterson et al., Org. Lett. 2004, 6, 1293-5; Crimmins et al., J. Am. Chem. Soc. 2002, 124, 5958-9; Williams et al., Tetrahedron Lett. 2002, 43, 4841-4; Nelson et al., 2002, 124, 13654-5; Uenishi et al., Angew. Chem. Int. Ed. 2005, 44, 2756-60; Gallagher et al., Bioorg. Med. Chem. Lett. 2004, 14, 575-9; Nadolski et al., Tetrahedron Lett. 2001, 41, 797-800; Messenger et al., Tetrahedron Lett. 2001, 42, 801-3; Sivaramakrishnan et al., Tetrahedron Lett. 2002, 43, 213-6; Lee et al., Bull. Korean Chem. Soc. 2001, 22, 791-792, 1179-1180; Lee et al., Bull. Korean Chem. Soc. 2003, 24, 1569-70; Shimizu et al., Tetrahedron Lett. 1997, 38, 6011-14; Shimizu et al., Synlett 1998, 1209-10; Mulzer et al., Chem. Rev. 2003, 103, 3753-86; Ghosh et al., Tetrahedron Lett. 2000, 41, 4705-8; Uenishi et al., Tetrahedron 2005, 61, 1971-9; Ahmed et al., J. Org. Chem. 2003, 68, 3026-42; and Gallagher et al., Tetrahedron Lett. 2005, 46, 923-36, and are incorporated herein by reference.

General Synthetic Strategy

The general synthetic strategy for the synthesis of laulimalide and laulimalide analogs, represented by compounds of Formula (I), is outlined below and in other embodiments is applied to any compound of Formulas (II), (III) or (IV), as defined herein. In some embodiments, compounds of Formula (III) are subjected to a cross-metathesis reaction and then processed to the desired laulimalide analog. In other embodiments, except when R³ is H, compounds of Formula (III) are reduced to compounds of Formula (IV). In yet further embodiments, compounds of Formula (IV) are also obtained by other methods. In further embodiments, compounds of Formula (IV) are subjected to a cross-metathesis reaction and then processed to the desired laulimalide analog. In some other embodiments, compounds of Formula (IV) are deprotected and oxidized to compounds of Formula (II). In yet further embodiments, compounds of Formula (II) are obtained by other methods. Compounds of Formula (II) are subjected to a cross-metathesis reaction to provide the desired laulimalide analog.

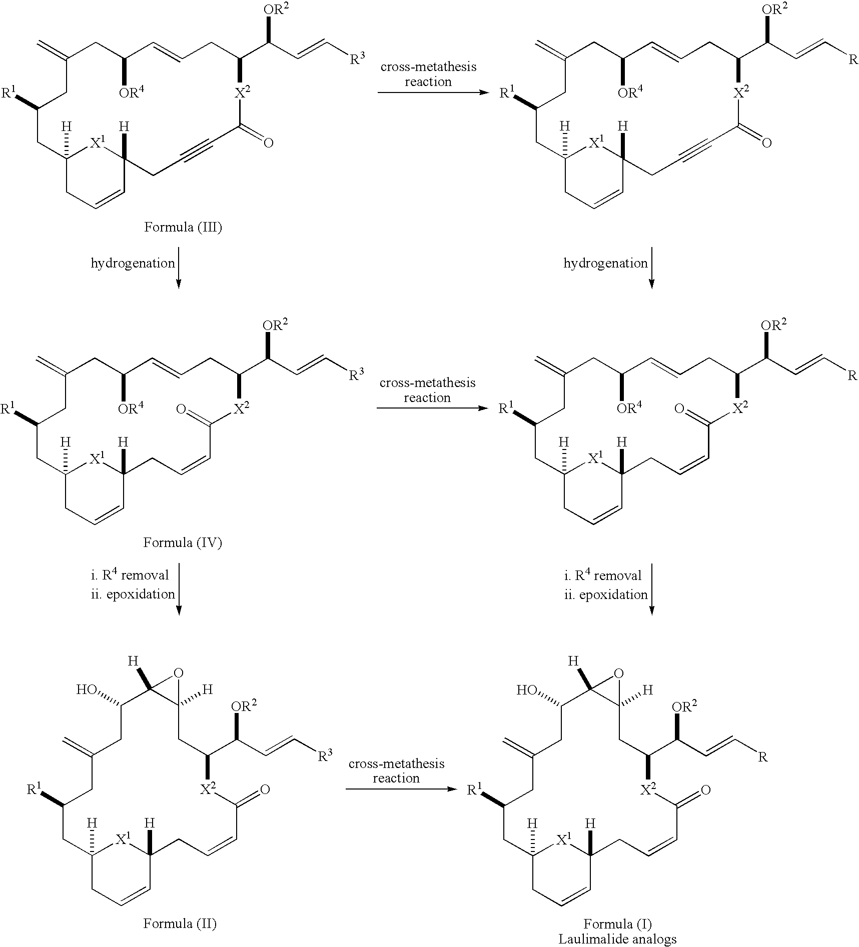

General Experimental Methods

Air- and moisture-sensitive reactions were carried out in oven-dried glassware sealed with rubber septa under a positive pressure of dry nitrogen or argon from a manifold or balloon, unless otherwise indicated. Stirring was provided by oven-dried Teflon-coated stir bars that were cooled under positive pressure of dry nitrogen or argon. Air- and moisture-sensitive liquid reagents, solvents, or solutions were transferred via syringe or stainless steel cannula under nitrogen or argon atmospheres.

Reaction temperatures refer to the temperature of the bath in which the reaction vessel was immersed. Room or ambient temperature refers to the temperature range of 20-25° C. Elevated temperatures were maintained using a silicone oil bath. Temperatures of 0 or −78° C. refer to ice/water and dry ice/acetone baths, respectively. A temperature of −20° C. maintained over a period longer than 1 hour refers to the placement of the reaction vessel in a standard freezer. A temperature of 4° C. refers to reactions run in a temperature-controlled cold room. Other temperatures below 20° C. were maintained within ±5° C. of the stated reaction temperature through careful monitoring and adjustments. Concentration of solutions in vacuo refers to evaporation using a Büchi rotary evaporator equipped with a Teflon seal vacuum pump. Residual solvents were removed from samples using a vacuum line held at 0.1-0.5 torr.

Reagents, unless otherwise indicated, were purchased from Aldrich Chemical Company and used as supplied without further purification. Tetrahydrofuran, toluene, diethyl ether, and dichloromethane were passed through alumina drying columns before use. All other solvents were taken from reagent grade or HPLC grade bottles, except when noted otherwise.

Analytical TLC was performed with 0.25 mm silica gel 60F plates with 254 nm fluorescent indicator from Merck. Plates were visualized by ultraviolet light and treatment with acidic p-anisaldehyde stain, potassium permanganate stain, or ceric ammonium molybdate stain followed by gentle heating. For all chromatographic purifications, silica gel 60, 230-400, purchased from EM, was used.

NMR spectra were measured on a Varian INOVA 600 (¹H at 600 MHz,), Varian INOVA 500 (¹H at 500 MHz), Varian XL-400 (¹H at 400 MHz), or Varian Gem-300 (¹H at 300 MHz) nuclear magnetic resonance spectrometer. Data for ¹H NMR spectra are reported as follows: chemical shift (δ ppm), multiplicity (s=singlet, d=doublet, t=triplet, q=quartet, dd=doublet of doublets, dt=doublet of triplets, ddd=doublet of doublet of doublets, dddd=doublet of doublet of doublet of doublets, br=broad), coupling constant (Hz), multiplicity. Chemical shift is reported relative to the residual solvent peak. Elemental analyses (% C, % H) were determined, by Desert Analytics, Tucson, Ariz. Reported atomic percentages are within error limits of ±0.4%. In instances were purity was not determined by elemental analysis, compounds displayed only one observable spot by TLC at the reported R_{f}.

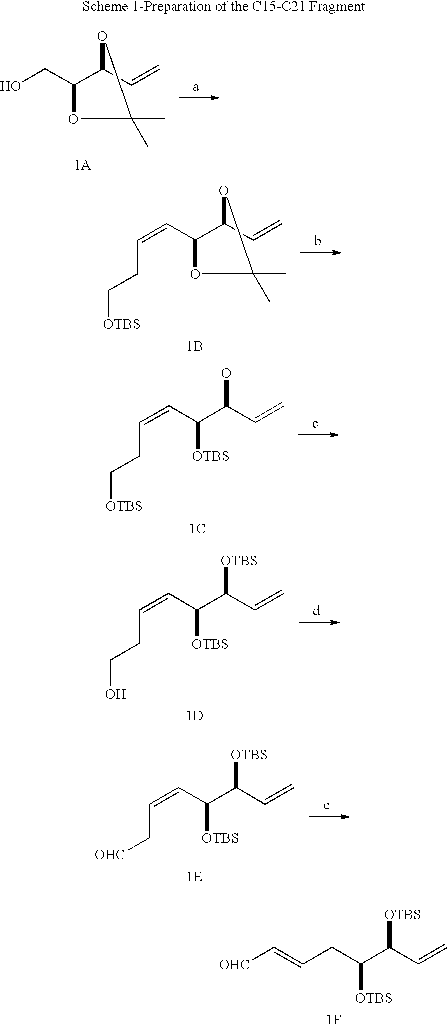

Step a: To a cold (−78° C.), stirred solution of oxalyl chloride (0.66 mL, 7.6 mmol) in dichloromethane (60 ml) was added DMSO (1.08 mL, 15.2 mmol) dropwise over 15 min. After the addition was complete, the reaction mixture was stirred for an additional 5 min and a solution of primary alcohol 1A (600 mg, 3.8 mmol, prepared by the procedure of Mukai et al., J. Chem. Soc., Perkin Trans. 1 1998, 2903-15) in CH₂Cl₂ (5 mL) was added dropwise over 10 min. The reaction mixture was stirred for −78° C. for 1 h, the Et₃N (3.17 mL, 23 mmol) was added dropwise over 10 min. The resulting yellowish solution was allowed to warm to room temperature over 1 h and was then diluted with Et₂O (100 mL). The organic layer was washed with 1N HCl (50 mL), saturated aqueous NaHCO₃ (50 mL), H₂O (50 mL), and brine (40 mL), then dried (MgSO₄), filtered, and concentrated in vacuo to provide a yellow oil that was co-evaporated with dry benzene (3×5 mL) under vacuum before being used directly in the next step without further purification.

To a cold (−30° C.), stirred suspension of TBSO(CH₂)₃PPh₃I (4.27 g, 7.6 mmol) in anhydrous THF (70 mL) and under positive pressure of N₂ was added NaHMDS (7.6 mL of a 1 M solution in THF, 7.6 mmol) dropwise over 30 min. During the course of the addition, the reaction mixture turned into a clear orange solution. This solution was stirred for an additional hour maintaining the cold bath between −30 and −20° C. A solution of the crude aldehyde in THF (5 mL) was added dropwise over 10 min. The resulting mixture was allowed to warm to room temperature over 2 h and then stirred for an additional 3 h. Saturated aqueous NH₄C (5 mL) was added, and the mixture was diluted with Et₂O (200 mL). The organic layer was washed with H20 (100 mL) and brine (80 mL), then dried (MgSO₄), filtered, and concentrated in vacuo. The residue was purified by flash chromatography (silica gel, Et₂O:pentane 1:30) to provide TBS ether 1B (843 mg, 71% over two steps) as a colorless oil. The product displayed only one observable spot by TLC at the reported R_{f} and in the solvent system used for chromatographic purification. ¹H NMR: (500 MHz, CDCl₃) δ 5.83 (ddd, J=17.5, 10.5, 7.5 Hz, 1H), 5.75 (dt, J=11.0, 7.5 Hz, Hz, 1H), 5.38 (br d, J=17.5 Hz, 1H), 5.26 (br d, J=10.5 Hz, 1H), 4.48 (t, J=8.5 Hz, 1H), 4.08 (t, J=7.5 Hz, 1H), 3.62 (t, J=7.5 Hz, 2H), 2.41-2.35 (m, 1H), 2.33-2.28 (m, 1H), 1.47 (s, 6(s, 6H), 0.91 (s, 9H), 0.07 (s, 6H).

Step b: To a stirred solution of 1B (700 mg, 2.24 mmol) in THF (40 mL) at room temperature under N₂ was added a 3N HCl solution (10 mL). The reaction mixture was stirred at room temperature for 24 h. Solid NaHCO₃ was added in small portions to quench the reaction until no gas formation was observed. The reaction mixture then was diluted with EtOAc (100 mL), dried with Na₂SO₄, filtered, and concentrated in vacuo. The residue was dissolved in CH₂Cl₂ (100 mL). This solution was then stirred under N₂ at −78° C. and Et₃N (1.55 mL, 11.1 mmol) was added dropwise over 5 min. The reaction mixture was stirred for 5 min and TBSOTf (2.05 mL, 8.9 mmol) was then added slowly over 15 min. After the addition was complete the reaction mixture was warmed to room temperature over 1 h and was then quenched by addition of saturated aqueous NH₄Cl solution (10 mL). The reaction mixture was diluted with Et₂O (200 mL) and washed with H₂O (100 mL) and brine (80 mL). The organic layer was dried with MgSO₄filtered, and concentrated in vacuo. The residue was purified by flash chromatography (silica gel, pentane) to give 1C (1.01 g, 90% over 2 steps) as a colorless oil. Atomic percentages as determined by combustion analysis were within ±0.4% of the expected values. ¹H NMR: (300 MHz, CDCl₃) δ 5.94 (ddd, J=17.5, 10.5, 5.0 Hz, 1H), 5.49 (dt, J=11.5, 7.0 Hz, 1H), 5.35 (m, 1H), 5.23 (dt, J=17.5, 1.8 Hz, 1H), 5.14 (dt, J=10.5, 1.8 Hz, 1H), 4.37 (dd, J=9.0, 5.5 Hz, 1H), 4.11 (br t, J=5.0 Hz, 1H), 3.66-3.62 (m, 2H), 2.40-2.34 (m, 1H), 2.33-2.27 (m, 1H), 0.93-0.84 (m, 27H), 0.11-0.01 (m, 18H).

Step c: To a stirred solution of tris-TBS ether 1C (733 mg, 1.46 mmol) in 2-propanol (25 mL) at room temperature and under positive pressure of N₂ was added cerium(IV) ammonium nitrate (802 mg, 1.46 mmol) in one portion. The resulting dark red solution was stirred at room temperature for 24 h, during which time the solution gradually turned light yellow. To this solution was added solid NaHCO₃ (200 mg). It was then diluted with Et₂O (200 mL), and the organic layer was washed with H₂O (2×80 mL) and brine (60 mL), then dried (MgSO₄), filtered, and concentrated in vacuo. The residue was purified by flash chromatography (silica gel, Et₂O:pentane 1:5) to provide primary alcohol 1D (558 mg, 99%) as a colorless oil. Atomic percentages as determined by combustion analysis were within ±0.4% of the expected values. ¹H NMR: (500 MHz, CDCl₃) δ 5.94 (ddd, J=17.5, 10.5, 5.0 Hz, 1H), 5.53-5.42 (m, 2H), 5.23 (dt, J=17.5, 2.0 Hz, 1H), 5.17 (dt, J=10.5, 2.0 Hz, 1H), 4.43 (dd, J=8.5, 4.5 Hz, 1H), 4.17 (ddt, J=5.0, 4.5, 2.0 Hz, 1H), 3.74-3.63 (m, 2H), 2.45-2.34 (m, 2H), 1.75 (t, J=6.0 Hz, 1H), 0.94 (s, 9H), 0.90 (s, 9H), 0.11 (s, 3H), 0.10 (s, 3H), 0.09 (s, 3H), 0.06 (s, 3H).

Step d: To a stirred solution of primary alcohol 1D (430 mg, 1.11 mmol) in dichloromethane (180 mL) at room temperature was added K₂CO₃ (768 mg, 5.6 mmol) followed by Dess-Martin periodinane (1.4 g, 3.4 mmol). The resulting milky suspension was stirred at room temperature for 5 h, then cooled to in an ice water bath (0° C.) and quenched by addition of a 1:1 mixture of saturated aqueous solutions of Na₂S₂O₃ and NaHCO₃ (80 mL). The resulting mixture was stirred vigorously at 0° C. to room temperature until the organic layer became clear (approximately 2 h). The mixture was then diluted with Et₂O (200 mL), and the organic layer was washed with H₂O (80 mL) and brine (80 mL), then dried (MgSO₄), filtered and concentrated in vacuo. The residue was purified by flash chromatography (silica gel, Et₂O:pentane 1:10) to provide aldehyde 1E (361 mg, 84%) as a colorless oil, The product displayed only one observable spot by TLC at the reported R_{f} and in the solvent system used for chromatographic purification. ¹H NMR: (400 MHz, CDCl₃) δ 9.67 (t, J=2.0 Hz, 1H), 5.88 (ddd, J=17.2, 10.4, 4.8 Hz, 1H), 5.72-5.65 (m, 1H), 5.58-5.53 (m, 1H), 5.21 (dt, J=17.2, 1.6 Hz, 1H), 5.14 (dt, J=10.4, 2.0 Hz, 1H), 4.31-4.27 (m, 1H), 4.12 (m, 1H), 3.28-3.24 (m, 2H), 0.89 (s, 9H), 0.87 (s, 9H), 0.06 (s, 3H), 0.05 (s, 3H), 0.04 (s, 3H), 0.01 (s, 3H).

Step e: To a stirred solution of aldehyde 1E (361 mg, 0.94 mmol) in CHCl₃ (passed through a pad of basic alumina before use, 50 mL) at room temperature under positive pressure of N₂ was added a solution of DBU (14 μL, 0.094 mmol) in CHCl₃ (2.5 mL) dropwise over 10 min. The resulting mixture was stirred at room temperature for 3.5 h, then quenched by addition of saturated aqueous NH₄Cl (5 mL). The biphasic mixture was diluted with Et₂O (100 mL) and washed with H₂O (40 mL) and brine (40 mL). The organic layer was dried (Na₂SO₄), filtered, and concentrated in vacuo. The residue was purified by flash chromatography (silica gel, Et₂O:pentane 1:3) to provide enal 1F (315 mg, 87%) as a yellow oil. The product displayed only one observable spot by TLC at the reported R_{f} and in the solvent system used for chromatographic purification. ¹H NMR: (500 MHz, CDCl₃) δ 9.51 (d, J=8.0 Hz, 1H), 6.89 (dt, J=15.5, 8.0 Hz, 1H), 6.12 (ddt, J=15.5, 8.0, 1.5 Hz, 1H), 5.99 (ddd, J=17.5, 11.0, 4.0 Hz, 1H), 5.33 (dt, J=17.5, 2.0 Hz, 1H), 5.23 (dt, J=11.0, 2.0 Hz, 1H), 4.23-4.21 (m, 1H), 3.76 (q. J=4.3 Hz, 1H), 2.62 (dddd, J=14.0, 7.5, 3.5, 1.5 Hz, 1H), 2.33 (dtd, J=14.5, 8.5, 1.5 Hz, 1H), 0.93 (s, 9H), 0.91 (s, 9H), 0.10 (s, 3H), 0.08 (s, 3H), 0.07 (s, 3H), 0.05 (s, 3H).

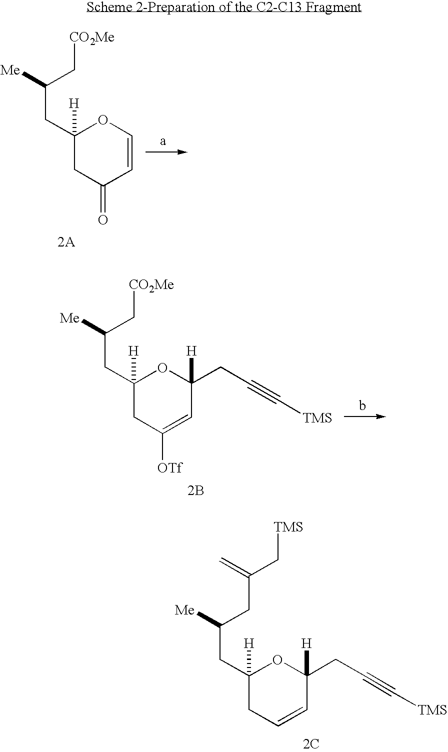

Step a: To a cooled (−78° C.), stirred solution of freshly distilled TMS-propyne (359 μL, 2.41 mmol) in dry THF (1 mL) under positive pressure of N₂ was added TMEDA (364 μL, 2.41 mmol) followed by n-BuLi (0.963 mL of a 2.5 M solution in hexanes, 2.41 mmol) dropwise over 2 min. The resulting mixture was stirred at −78° C. for 30 min then warmed to −20° C. and stirred at this temperature for an additional hour. It was then cooled to −78° C. and transferred by cannula into a cooled (−78° C.) suspension of CuI (459 mg, 2.41 mmol, recrystallized) in THF (1.5 mL). This mixture was stirred at −78° C. for 15 min then warmed to −20° C. and stirred at this temperature for 1 h. The resulting slurry was cooled to −78° C. and a solution of pyranone 2A (146 mg, 0.69 mmol) in THF (1 mL) was added dropwise over 5 min. The resulting mixture was stirred at −78° C. for 1 h then warmed to −20° C. and stirred at this temperature for 2 h. The solution was then cooled to −78° C. and a solution of Comins' reagent (945 mg, 2.41 mmol) in THF (1.5 mL) was added dropwise over 5 min. The reaction mixture was stirred for 2 h at −78° C. and showed to slowly warm to rt over 12 hours. The mixture was then cooled to −78° C. and quenched by addition of saturated aqueous NH₄Cl (5 mL). The resulting biphasic mixture was diluted with Et₂O (30 mL), and the organic layer was washed with a 2:1 mixture of saturated aqueous NH₄Cl and NH₄OH (10 mL), H₂O (10 mL), and brine (10 mL). The organic layer was dried (MgSO₄), filtered, and concentrated in vacuo to afford a crude oil that was purified by flash chromatography (silica gel, pentane:Et₂O 10:1) to provide enol trilate 2B (245 mg, 78%, dr=95:5) as a colorless oil. The product displayed only one observable spot by TLC at the reported R_{f} and in the solvent system used for chromatographic purification. ¹H NMR: (500 MHz, CDCl₃) δ 5.95-5.94 (m, 1H), 4.54-4.48 (m, 1H), 4.07-4.00 (m, 1H), 3.70 (s, 3H), 2.60 (dd, J=16.7, 6.4 Hz, 1H), 2.53 (dd, J=16.7, 7.5 Hz, 1H), 2.35-2.18 (m, 5H), 1.69 (ddd, J=14.1, 10.2, 4.1 Hz, 1H), 1.33-1.22 (m, 1H), 0.99 (d, J=6.2 Hz, 3H), 0.14 (s, 9H).

Step b: To a solution of enol triflate 2B (194 mg, 0.43 mmol) in THF (2.5 mL) at room temperature and under positive pressure of N₂ was added LiCl (140 mg, 3.3 mmol) followed by Pd(PPh₃)₄ (10 mg, 0.0085 mmol). Tributyltin hydride (173 mg, 0.60 mmol) was added dropwise over 5 minutes and the resulting orange solution was stirred at room temperature for 30 minutes. The solvents were evaporated in vacuo and the crude oil was purified by flash chromatography (silica gel, pentane:Et₂O 10:1) to afford the reduced product (131 mg, >100%, contaminated with tin byproducts) of a yellow oil that was carried on without further purification. R_{f}=0.52 (pentane:Et₂O 5:1). An oven-dried flask was equipped with a stir bar and charged with powdered CeCl₃ (1.26 g, 5.1 mmol, Aldrich +99.99%). The flask was placed in a vacuum oven at 130° C. for 2 hours then 150° C. for 16 hours. The flask was cooled under vacuum, placed under positive pressure of N₂, then dissolved in anhydrous THF (4 mL). This mixture was stirred vigorously at room temperature for 24 h. The resulting fine milky white suspension was cooled to −78° C. and TMSCH₂MgCl (5.1 mL of a 1 M solution in diethyl ether, Fluka, 5.1 mmol) was added dropwise over 5 min. The resulting slightly yellowish suspension was stirred for 3 h at −78° C. A solution of the crude reduced enol triflate (131 mg) in THF (2 mL) was added dropwise to this mixture over 5 minutes. The resulting slurry was stirred at −78° C. for 2 hours, then allowed to slowly warm to rt and stirred at this temperature for 16 h. It was then quenched by addition of saturated aqueous NH₄Cl (5 mL). The resulting mixture was diluted with diethyl ether (50 mL), and the organic layer was washed with H₂O (20 mL), dried (MgSO₄), filtered, and concentrated in vacuo to provide a yellow oil (325 mg). This oil was dissolved in CH₂Cl₂ (10 mL) and silica gel (2 g) was added. This mixture was stirred at room temperature and under N₂ for 16 hours, then filtered and concentrated in vacuo. The residue was purified by flash chromatography (silica gel, pentane) to afford allyl silane 2C (132 mg, 86% over two steps) as a colorless oil. Atomic percentages as determined by combustion analysis were within ±0.4% of the expected values. ¹H NMR: (500 MHz, CDCl₃) δ 5.90-5.85 (m, 1H), 5.84-5.80 (m, 1H), 4.57 (br s, 1H), 4.54 (br s, 1H), 4.35-4.30 (m, 1H), 3.84-3.78 (m, 1H), 2.55 (dd, J=16.6, 6.9 Hz, 1H), 2.46 (dd, J=16.6, 6.9 Hz, 1H), 2.02-1.86 (m, 4H), 1.80 (dd, J=13.5, 7.9 Hz, 1H), 1.66-1.60 (m, 1H), 1.50 (br s, 2H), 1.07 (ddd, J=13.5, 9.9, 3.1 Hz, 1H), 0.89 (d, J=6.5 Hz, 3H), 0.13 (s, 9H), 0.01 (s, 9H).

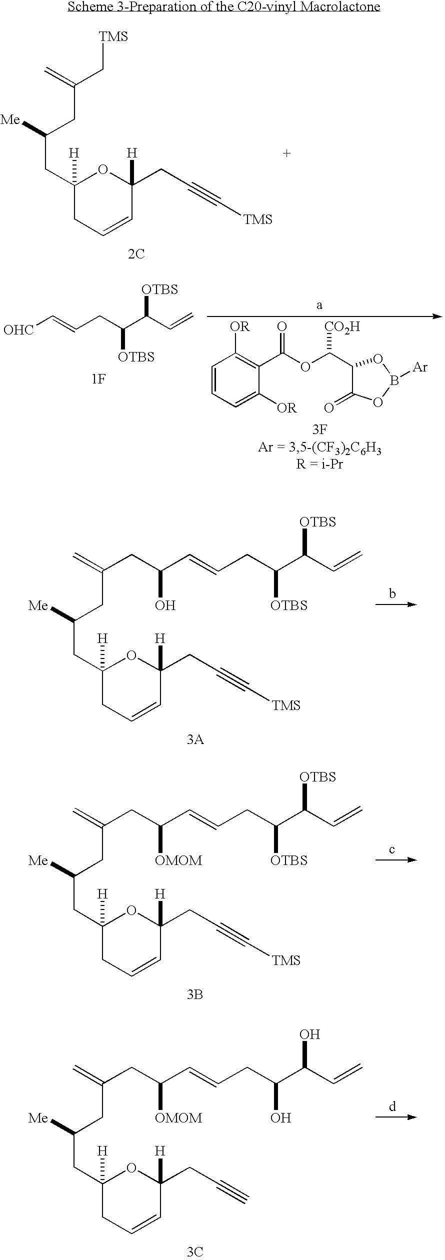

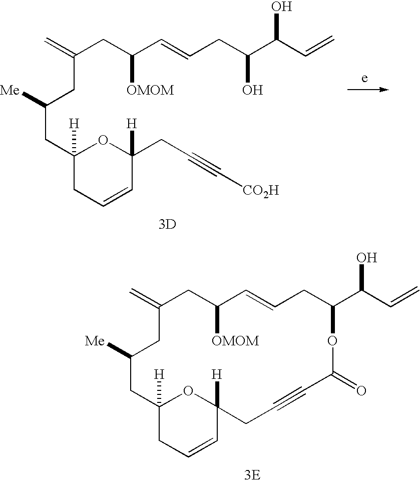

Step a: The D-tartaric acid derived CAB ligand (272 mg, 0.73 mmol) was dried in a vacuum oven (60° C.) for 24 hours prior to use. The dry ligand was dissolved in propionitrile (1.5 mL, freshly distilled from CaH₂). To this stirred solution under positive pressure of N₂ was added 3,5-bis(trifluoromethyl)-phenylboronic acid (Lancaster, 190 mg, 0.73 mmol) in one portion. The mixture was stirred at room temperature for 10 hours, to provide complex 3F. The solution was cooled to −78° C. and a solution of enal 1F (160 mg, 0.416 mmol) and allyl silane 2C (200 mg, 0.551 mmol) in freshly distilled propionitrile (0.15 mL) was added slowly down the side of the reaction flask to the CAB complex at −78° C. over 5 minutes, followed by a propionitrile wash (0.15 mL). The reaction mixture was stirred at −78° C. for 56 hours, then quenched by addition of saturated aqueous NaHCO₃ (2 mL). The mixture was allowed to warm slowly to room temperature over 2 h, then diluted with Et₂O (60 mL). The organic layer was washed with H₂O (10 mL) and brine (10 mL), then dried (Na₂SO₄), filtered, and concentrated in vacuo. The crude residue was purified by flash chromatography (silica gel, ethyl acetate:pentane 1:19) to provide allylic alcohol 3A (194 mg, 69%, 95:5 dr) as a colorless oil. The product displayed only one observable spot by TLC at the reported R_{f} and in the solvent system used for chromatographic purification. ¹H NMR: (500 MHz, CDCl₃) δ5.99 (ddd, J=17.5, 10.5, 4.0 Hz, 1H), 5.92-5.84 (m, 2H), 5.71 (ddt, J=15.0, 7.5, 1.0 Hz, 1H), 5.49 (dd, J=15.0, 6.5 Hz, 1H), 5.28 (dt, J=17.5, 2.2 Hz, 1H), 5.16 (dt, J=10.5, 2.2 Hz, 1H), 4.90 (m, 2H), 4.33 (m, 1H), 4.18-4.14 (m, 2H), 3.82 (m, 1H), 3.60 (ddd, J=9.5, 4.5, 3.0 Hz, 1H), 2.56 (dd, J=16.5, 6.5 Hz, 1H), 2.49 (dd, J=16.5, 7.5 Hz, 1H), 2.33-2.29 (m, 1H), 2.25 (dd, J=14.0, 4.0 Hz, 1H), 2.16 (dd, J=14.0, 9.5 Hz, 1H), 2.07 (dd, J=13.5, 5.5 Hz, 1H), 2.00-1.93 (m, 4H), 1.87 (dd, J=13.5, 8.5 Hz, 1H), 1.75 (d, J=3.0 Hz, 1H), 1.63 (ddd, J=14.0, 10.0, 4.0 Hz, 1H), 1.14 (ddd, J=14.5, 9.5, 3.0 Hz, 1H), 0.92-0.89 (m, 21H), 0.15 (s, 9H), 0.07 (s, 3H), 0.06 (s, 6H), 0.05 (s, 3H).

Step b: To a stirred solution of allylic alcohol 3A (810 mg, 1.20 mmol) in dichloromethane (8 mL) at 0° C. and under positive pressure of N₂ was added diisopropylethylamine (2.09 mL, 12.0 mmol) over 5 minutes followed by chloromethyl methyl ether (0.82 mL, 10.8 mmol) dropwise over 5 minutes. The reaction mixture was warmed to room temperature over 10 min and stirred at that temperature for 20 hours, then diluted with Et₂O (300 mL) and washed with 1N HCl (50 mL), saturated aqueous NaHCO₃ (50 mL), H₂O (50 mL), and brine (50 mL). The organic layer was dried (Na₂SO₄), filtered, and concentrated in vacuo. The residue was purified by flash chromatography (silica gel, EtOAc:pentane 1:19) to provide MOM ether 3B (859 mg, 99.5%) as a colorless oil. The product displayed only one observable spot by TLC at the reported R_{f} and in the solvent system used for chromatographic purification. ¹H NMR: (500 MHz, CDCl₃) δ 5.97 (ddd, J=17.5, 10.5, 4.0 Hz, 1H), 5.92-5.87 (m, 1H), 5.86-5.82 (m, 1H), 5.67 (dt, J=15.0, 7.5 Hz, 1H), 5.33-5.30 (m, 1H), 5.25 (dt, J=17.5, 2.0 Hz, 1H), 5.15 (dt, J=10.5, 2.0 Hz, 1H), 4.86 (br s, 1H), 4.81 (br s, 1H), 4.68 (d, J=6.5, 3.0 Hz, 1H), 4.48 (dd, J=6.5, 3.5 Hz, 1H), 4.31 (br t, J=6.5 Hz, 1H), 4.15-4.09 (m, 2H), 3.81 (ddd, J=14.0, 7.5, 3.5 Hz, 1H), 3.57 (ddd, J=8.0, 5.0, 3.5 Hz, 1H), 3.34 (s, 3H), 2.55 (dd, J=16.5, 6.5 Hz, 1H), 2.48 (dd, J=16.5, 7.5 Hz, 1H), 2.34-2.29 (m, 2H), 2.17 (dd, J=14.0, 5.5 Hz, 1H), 2.06-1.87 (m, 6H), 1.63 (ddd, J=14.0, 9.5, 4.0 Hz, 1H), 1.11 (ddd, J=14.0, 9.5, 3.5 Hz, 1H), 0.91 (s, 9H), 0.89 (s, 9H), 0.88 (m, 3H), 0.14 (s, 9H), 0.06 (s, 3H), 0.05 (s, 3H), 0.04 (s, 3H), 0.03 (s, 3H).

Step c: To a solution of MOM ether 3B (305 mg, 0.424 mmol) in THF (8 mL) at 0° C. was added TBAF (1.48 mL of a 1.0 M solution in THF, 1.48 mmol) dropwise over 5 minutes. The resulting solution was allowed to stir for 1 h, then was removed from the ice bath and stirred at room temperature for 2 h. The reaction was then diluted with ethyl acetate (100 mL), and the organic layer was washed with water (50 mL) and brine (50 mL), then dried (Na₂SO₄), filtered, and concentrated in vacuo. Flash chromatography (silica gel, pentane:ethyl acetate 1:1) gave diol 3C (160 mg, 90%) as a yellow oil. The product displayed only one observable spot by TLC at the reported R_{f}. ¹H NMR: (500 MHz, CDCl₃) δ 5.92-5.81 (m, 3H), 5.69-5.62 (m, 1H), 5.46 (ddt, J=15.5, 8.0, 1.0 Hz, 1H), 5.36 (dt, J=17.5, 1.5 Hz, 1H), 5.24 (dt, J=10.5, 1.5 Hz, 1H), 4.83 (bs, 1H), 4.81 (bs, 1H), 4.69 (d, J=7.0 Hz, 1H), 4.59 (d, J=7.0 Hz, 1H), 4.35-4.30 (m, 1H), 4.15-4.08 (m, 1H), 3.98-3.93 (m, 1H), 3.83-3.76 (m, 1H), 3.55-3.49 (m, 1H), 3.35 (s, 3H), 2.76 (d, J=4.0 Hz, 1H), 2.54-2.48 (m, 2H), 2.43 (ddd, J=16.5, 7.0, 2.5 Hz, 1H), 2.39-2.30 (m, 2H), 2.23-2.14 (m, 2H), 2.06-1.84 (m, 6H), 1.64-1.58 (m, 1H), 1.14-1.07 (m, 1H), 0.88 (d, J=6.5 Hz).

Step d: To a solution of terminal alkyne 3C (160 mg, 0.382 mmol), pre-dried by evaporating from benzene (3×3 mL) in dry THF (3.5 mL) in a −78 ° C. bath was added n-BuLi (1.66 mL of a 1.38 M solution in THF, 2.29 mmol) dropwise over 5 minutes. The resulting yellow solution was allowed to stir at −78 ° C. for 2 minutes, then solid CO₂ (approx. 500 mg) was added. The reaction mixture became very viscous, and slowly turned dark orange over 5 min. The solution was stirred for an additional 5 min, over which time the orange color slowly disappeared, then was quenched by addition of saturated aqueous NH₄Cl (3 mL). The mixture was removed from the cold bath and allowed to warm to room temperature, then diluted with ethyl acetate (80 mL) and 1M NaH₂PO₄ (10 mL). The aqueous layer was extracted with ethyl acetate (3×10 mL), and the combined organic fractions were dried (Na₂SO₄), filtered, and concentrated. The residue was purified by flash chromatography (silica gel, 1:3 MeOH:CHCl₃) to provide carboxylic acid 3D (166 mg, 94%) as an oily white solid. ¹H NMR: (500 MHz, CDCl₃) δ 5.89-5.76 (m, 3H), 5.75-5.66 (m, 1H), 5.43 (dd, J=15.5, 7.5 Hz, 1H), 5.34 (d, J=17.5 Hz, 1H), 5.22 (d, J=10.5 Hz, 1H), 4.84 (s, 1H), 4.79 (s, 1H), 4.69 (d, J=7.0 Hz, 1H), 4.52 (d, J=7.0 Hz, 1H), 4.38-4.30 (m, 1H), 4.21-4.14 (m, 1H), 4.06-3.98 (m, 1H), 3.80-3.71 (m, 1H), 3.69-3.61 (m, 1H), 3.33 (s, 3H), 2.56 (dd, J=17.0, 8.0 Hz, 1H), 2.44 (dd, J=17.0, 6.0 Hz, 1H), 2.40-2.32 (m, 1H), 2.32-2.16 (m, 4H), 2.05-1.96 (m, 1H), 1.95-1.85 (m, 2H), 1.84-1.77 (m, 1H), 1.67-1.58 (m, 1H), 1.16-1.08 (m, 1H), 0.85 (d, J=6.5 Hz, 3H).

Step e: To a solution of carboxylic acid 3D (36 mg, 0.078 mmol) in anhydrous benzene (210 mL) at room temperature and under positive pressure of N₂ was added triethylamine (0.17 mL, 1.206 mmol) in one portion followed by 2,4,6-trichlorobenzoyl chloride (94 μL, 0.603 mmol) dropwise over 2 minutes. The resulting solution was allowed to stir for 30 minutes, and then DMAP (124 mg, 1.012 mmol) was added in one portion. The resulting cloudy yellow solution was stirred at room temperature for 1 hour, and then saturated aqueous NaHCO₃ (60 mL) was added. The resulting biphasic mixture was stirred vigorously for 2 hours. The organic layer was separated and the aqueous layer was back—extracted with ethyl acetate (3×30 mL). The combined organic fractions were dried (Na₂SO₄), filtered, and concentrated in vacuo. The residue was purified by flash chromatography (silica gel, 1:2 ethyl acetate:pentane) to provide macrolactone 3E (15 mg, 43%) as an off-white solid. The product displayed only one observable spot by TLC at the reported R_{f} and in the solvent system used for chromatographic purification. ¹H NMR: (500 MHz, CDCl₃) δ 5.94-5.89 (m, 1H), 5.85 (ddd, J=17.5, 10.5, 6.0 Hz, 1H), 5.69-5.59 (m, 2H), 5.54 (dd, J=15.5, 6.5 Hz, 1H), 5.38 (dt, J=17.0, 1.5 Hz, 1H), 5.27 (dt, J=10.5, 1.5 Hz, 1H), 5.07-5.02 (m, 1H), 4.87 (s, 1H), 4.80 (s, 1H), 4.65 (d, J=7.0 Hz, 1H), 4.49 (d, J=7.0 Hz, 1H), 4.47-4.41 (m, 1H), 4.26-4.21 (m, 1H), 4.20-4.15 (m, 1H), 3.73-3.66 (m, 1H), 3.31 (s, 3H), 2.72 (dd, J=17.5, 11.5 Hz, 1H), 2.44-2.30 (m, 3H), 2.25-2.12 (m, 3H), 2.02-1.88 (m, 4H), 1.84 (dd, J=13.0, 10.0 Hz, 1H), 1.63-1.55 (m, 1H), 1.13-1.06 (m, 1H), 0.84 (d, J=6.0 Hz, 3H).

Alternatively, compound 3B may be transformed into compound 3D by the methods described below.

To a stirred solution of TMS-alkyne 3B (23 mg, 0.032 mmol) in methanol (1 mL) at room temperature and under positive pressure of N₂ was added in one portion K₂CO₃ (13.3 mg, 0.096 mmol). The reaction mixture turned cloudy and yellow after a few minutes. Following stirring at room temperature for 3 hours the reaction mixture was diluted with Et₂O (20 mL). The organic layer was washed with H₂O (5 mL) and brine (5 mL), then dried (Na₂SO₄), filtered, and concentrated in vacuo. The residue was purified by flash chromatography (silica gel, pentane: Et₂O 10:1) to provide the terminal alkyne (20 mg, 96%) as a colorless oil. The product displayed only one observable spot by TLC at the reported R_{f} and in the solvent system used for chromatographic purification.

To a solution of the terminal alkyne prepared above (140 mg, 0.216 mmol) in anhydrous THF (2.5 mL) in a −78° C. bath was added n-BuLi (0.55 mL of a 1.6 M solution in hexanes, 0.88 mmol) dropwise over 5 min. To the resulting bright yellow solution was added solid CO₂ (approx. 300 mg). This mixture was stirred at −78° C. for 5 min then quenched by addition of saturated aqueous NH₄Cl (4 mL). The flask was then removed from the cold bath and allowed to warm to room temperature. It was diluted with EtOAc (30 mL) and water (2 mL). The aqueous layer was separated and acidified to pH 2 with 1N HCl, then extracted with EtOAc (5 mL). The combined organic fractions were dried over Na₂SO₄, filtered, and concentrated in vacuo. Flash chromatography (silica gel, MeOH:CHCl₃ 1:7) gave the carboxylic acid (130 mg, 87%) as a yellow oil. The product displayed only one observable spot by TLC at the reported R_{f} and in the solvent system used for chromatographic purification.

To a solution of the carboxylic acid prepared above (110 mg, 0.159 mmol) in THF (8.5 mL) in a polypropylene vial equipped with a stir bar and under positive pressure of N₂ was added HF pyridine (1.7 mL of a 20% stock solution in THF) dropwise over 10 min. The vial was capped and the resulting solution was allowed to stir at room temperature for 9 days, then quenched by first diluting with EtOAc (25 mL) and then adding solid NaHCO₃ until no further gas evolution was observed. To this mixture was added 1M NaH₂PO₄ (8 mL), which brought the pH of the aqueous layer to approximately 5. The aqueous layer was separated and back extracted with EtOAc (2×5 mL). The combined organic fractions were dried (Na₂SO₄), filtered, and concentrated in vacuo. The crude residue was purified by flash chromatography (silica gel, MeOH:CHCl₃ 1:4) to provide diol acid 3D (73 mg, 99%) as an off-white amorphous solid.

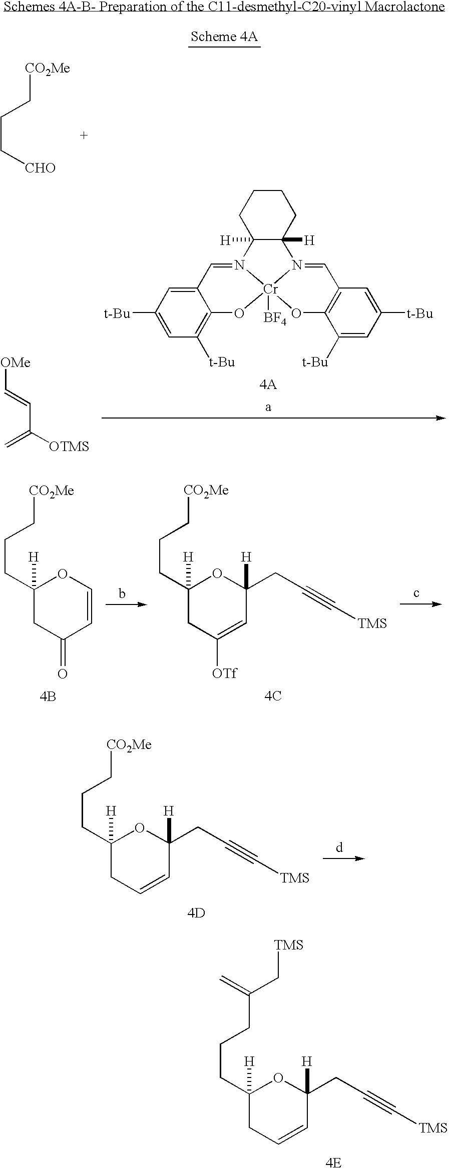

Step a: To a solution of methyl 5-oxopentanoate (prepared following the procedure of Huckstep et al., Synthesis 1982, 881-2, 650 mg, 5 mmol) in t-butyl methyl ether (1.7 mL) at 0° C. under N₂ was added activated 4 Å molecular sieves (1.5 g, 0.3g/mmol of aldehyde) that had been cooled under positive pressure of N₂. The resulting white slurry was stirred for 30 min at 0° C. then cooled to −78° C. for 15 min. (S,S)—Cr-Salen catalyst-BF₄⁺4A (0.135 g, 0.19 mmol) was added and the reaction mixture stirred an additional 5 min at −78° C. The reaction had become red-orange in color and very difficult to stir. Danishefsky's diene (1.55 g, 9 mmol) was added dropwise over 5 min, and the reaction was allowed to stir for an additional hour at −78° C. The reaction was quickly warmed to −20° C. and stirred at this temperature for 18 hr, then cooled back to −78° C. and treated sequentially with CH₂Cl₂ (30 mL) and TFA (0.25 mL). The mixture was quickly warmed to room temperature and stirred for 2.5 h, then filtered through a pad of silica gel, eluting with EtOAc (5×40 ml). The resulting eluant was reduced in vacuo to provide a dark red oil. Purification of the crude residue by flash chromatography (silica, 75:25 petroleum ether:Et₂O) afforded pyranone 4B (707 mg, 71%, 84% ee) as a red oil. The product displayed only one observable spot at the reported R_{f} and in the solvent system used for chromatography. ¹H NMR: (500 MHz, CDCl₃) δ 7.34 (d, J=6.1 Hz, 1H), 5.40 (dd, J=6.1, 1.2 Hz, 1H), 4.44-4.37 (m, 1H, C9-H), 3.68 (s, 3H), 2.56-2.36 (m, 4H), 1.89-1.69 (m, 4H).

Step b: To a cold (−78° C.) solution of 1-TMS-1-propyne (1.23 mL, 8.32 mmol) and TMEDA (1.37 mL, 9.07 mmol) in THF (7.0 mL) under N₂ was added n-BuLi (4.73 mL of a 1.6 M solution in hexane, 7.56 mmol) dropwise over 10 min. The solution was stirred at −78° C. for 30 min, then warmed to −20° C. and stirred for 30 min before being cooled to −78° C. and transferred by cannula into a cold (−78° C.) suspension of freshly recrystallized CuI (1.58 g, 8.32 mmol) in THF (2.0 mL). The resulting mixture was stirred at −78° C. for 15 min then warmed to −20° C. and stirred 1 h at this temperature. The resulting slurry was cooled to −78° C. and a solution of pyranone 4B (500 mg, 2.52 mmol) in THF (2.0 mL) was added dropwise over 5 min. The reaction mixture was stirred at −78° C. for 1 h and −20° C. for 15 min. The reaction mixture was then cooled to −78° C. and a solution of Comins' reagent (3.46 g, 8.82 mmol) in THF (7 ml) was added dropwise over 10 min. The resulting suspension was allowed to warm slowly to rt over 15 h. The mixture was quenched with NH₄Cl aq. (5 mL) and diluted with Et₂O (100 mL). The organic layer was separated and washed with water (50 mL), a 1:1 solution of NH₄Cl:NH₄OH (50 mL), H₂O (50 mL), and brine (50 ml), dried over MgSO₄, filtered, and the solvent evaporated in vacuo. The resulting oil was purified using flash chromatography (silica gel, pentane:ether, 95:5 then 85:15) to afford 780 mg (70%, >95:5 dr) of enol triflate 4C as a pale yellow oil. Atomic percentages as determined by combustion analysis were within ±0.4% of expected values. ¹H NMR: (500 MHz, CDCl₃) δ 5.97-5.96 (m, 1H), 4.52-4.49 (m, 1H), 3.87 (m, 1H), 3.67 (s, 3H), 2.60 (dd, J=16.6, 6.3 Hz, 1H), 2.51 (dd, J=16.6, 7.7 Hz, 1H), 2.35-2.25 (m, 4H), 1.87-1.79 (m, 1H), 1.73-1.51 (m, 3H), 0.14 (s, 9H).

Step c: To a solution of enol triflate 4C (297 mg, 0.67 mmol) in THF (5.2 mL) at room temperature and under N₂ was added LiCl (222 mg, 5.23 mmol) followed by Pd(PPh₃)₄ (15.5 mg, 0.0134 mmol). To this heterogeneous mixture was then added tributyltin hydride (0.25 mL, 0.938 mmol) dropwise over 5 min, being careful to maintain the temperature of the solution at rt. The resulting mixture was stirred for 25 min, then diluted with EtOAc (6 mL) and treated with 1.0 M aqueous KF (0.15 mL). The resulting solution was stirred at rt for 30 min, then filtered through a pad of Celite, eluting with EtOAc (5×10 mL). The filtrate was dried (MgSO₄), filtered, and concentrated in vacuo. The yellow, oily residue was purified by flash chromatography (SiO₂, 15:85 Et₂O:pentane) to provide ester 4D (180 mg, 91%) as a pale yellow oil. The product displayed only one observable spot by TLC at the reported R_{f} and in the solvent system used for chromatography. ¹H NMR: (500 MHz, CDCl₃) δ 5.90-5.82 (m, 2H), 4.33-4.29 (m, 1H), 3.70-3.65 (m, 1H), 3.66 (s, 3H), 2.54 (dd, J=16.6, 7.0 Hz, 1H), 2.45 (dd, J=16.5, 7.3 Hz, 1H), 2.37-2.33 (m, 2H), 2.03-1.78 (m, 2H), 1.75-1.50 (m, 2H), 1.49-1.18 (m, 2H), 0.14 (s, 9H).

To a flask equipped with a stir bar and powdered cerium (III) chloride (3.79 g, 15.36 mmol, pre-dried in a vacuum oven at 120° C. overnight before use) at room temperature and under N₂ was added THF (10 mL) gradually over 10 minutes. The resulting suspension was stirred at room temperature for 24 h. The resulting white slurry was cooled to −78° C. and TMSCH₂MgCl (15.36 mL of a 1.0 M solution in diethyl ether, 15.36 mmol) was added dropwise over 10 min. The resulting yellow mixture was stirred at −78° C. for 2 h, then a solution of ester 4D (376 mg, 1.28 mmol) in THF (2 mL) was added dropwise over 5 min, followed by a THF wash (2 mL). The reaction mixture was stirred at −78° C. for 2 h, then warmed slowly to room temperature and allowed to stir overnight (18 h). The mixture was cooled to 0° C. and quenched by slow addition of a saturated aqueous solution of Rochelle's salt (100 mL), removed from the ice bath and allowed to stir at room temperature for 2 h, then extracted with Et₂O (3×150 mL). The combined organic fractions were washed with brine (100 mL), then dried over MgSO₄, filtered, and concentrated in vacuo to give a crude yellow oil (1.58 g). This oil was dissolved in CH₂Cl₂ (17 mL) and silica gel (1.4 g) was added. The resulting suspension was stirred at room temperature under N₂ for 24 h, then filtered and concentrated. The crude oil was purified by flash chromatography (silica gel, 95:5 pentane:Et₂O) to give allylsilane 4E (440 mg, 99%) as a clear oil. The product displayed only one observable spot by TLC at the reported R_{f} and in the solvent system used for chromatographic purification. ¹H NMR: (400 MHz, CDCl₃) δ 5.90-5.81 (m, 2H), 4.59 (br s, 1H), 4.51 (br s, 1H), 4.35-4.30 (m, 1H), 3.70-3.64 (rm, 1H), 2.55 (dd, J=16.6, 6.9 Hz, 1H), 2.46 (dd, J=16.6, 6.9 Hz, 1H), 2.04-1.87 (m, 4H), 1.68-1.25 (m, 4H), 1.59 (br s, 2H), 0.14 (s, 9H), 0.01 (s, 9H).

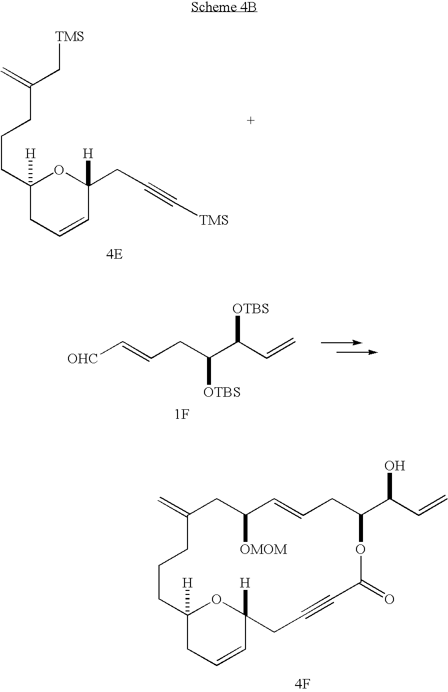

The C11-desmethyl-C20-vinyl Macrolactone is prepared following the procedure set forth in Scheme 3 substituting allylsilane 2C with allylsilane 4E.

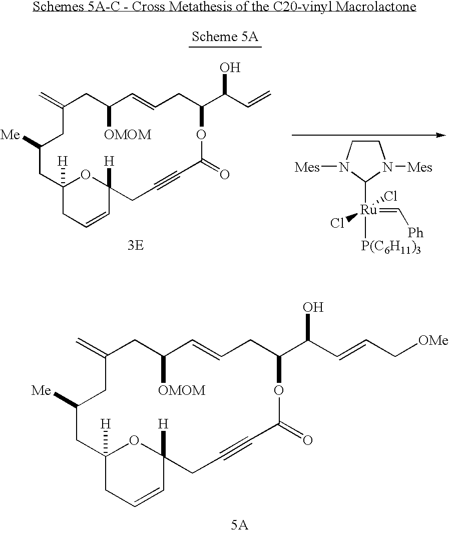

A solution of macrolactone 3E (12 mg, 0.027 mmol) in CH₂Cl₂ (4 mL) was degassed using the freeze-pump-thaw method. To this solution at room temperature and under positive pressure of N₂ was added allyl methyl ether (54 μL, 0.54 mmol) followed by Grubbs' 2nd generation metathesis catalyst (2.3 mg, 0.0027 mmol). The resulting solution was allowed to stir at room temperature for 24 h. The solvent was removed in vacuo, and the residue was purified via flash chromatography (silica gel, EtOAc:pentane 1:1) to provide recovered 3E (7.4 mg) and cross metathesis product 5A (4.8 mg, 36%, >99% borsm) as a yellow oil. The product displayed only one observable spot by TLC at the reported R_{f}. ¹H NMR: (500 MHz, CDCl₃) δ 5.94-5.86 (m, 2H), 5.76-5.69 (m, 1H), 5.68-5.60 (m, 2H), 5.53 (dd, J=15.5, 7.0 Hz, 1H), 5.06-5.00 (m, 1H), 4.87 (s, 1H), 4.80 (s, 1H), 4.65 (d, J=7.0 Hz, 1H), 4.48 (d, J=7.0 Hz, 1H), 4.47-4.41 (m, 1H), 4.26-4.17 (m, 2H), 3.95 (d, J=5.5 Hz, 2H), 3.73-3.66 (m, 1H), 3.33 (s, 3H), 3.31 (s, 3H), 2.71 (dd, J=17.5, 11.5 Hz, 1H), 2.42-2.27 (m, 4H), 2.26-2.12 (m, 3H), 2.03-1.88 (m, 3H), 1.84 (d, J=13.5, 10.0 Hz, 1H), 1.86-1.82 (m, 1H), 1.13-1.06 (m, 1H), 0.83 (d, J=6.0 Hz, 3H).

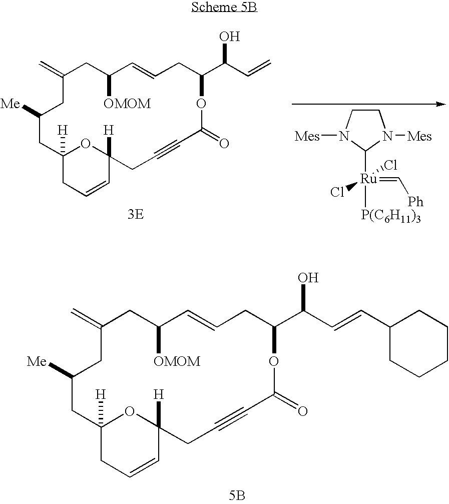

A solution of macrolactone 3E (10 mg, 22.5 μmol) in CH₂Cl₂ (3 mL) was degassed using the freeze-pump-thaw method. To this solution equipped with a Teflon stir bar at room temperature was added vinylcyclohexane (62 μL, 0.45 mmol) followed by the Grubbs second-generation catalyst (1.9 mg, 2.25 μmol). The resulting solution was stirred at room temperature for 24 h. The solvent was evaporated and the residue purified by flash chromatography (silica gel, ethyl acetate:pentane 1:3) to provide metathesis product 5B (8 mg, 68%) as a yellow oil. The product displayed only one observable spot by TLC at the reported R_{f} and in the solvent system used for chromatographic purification. ¹H NMR: (500 MHz, CDCl₃) δ 5.94-5.89 (m, 1H), 5.72 (ddd, J=15.5, 6.5, 2.0 Hz, 1H), 5.68-5.60 (m, 2H), 5.52 (dd, J=16.0, 7.0 Hz, 1H), 5.38 (ddd, J=16.0, 7.0, 1.5 Hz, 1H), 5.02-4.96 (m, 1H), 4.87 (s, 1H), 4.80 (s, 1H), 4.65 (d, J=7.0 Hz, 1H), 4.48 (d, J=7.0 Hz, 1H), 4.47-4.41 (m, 1H), 4.26-4.20 (m, 1H), 4.11-4.06 (m, 1H), 3.73-3.66 (m, 1H), 3.31 (s, 3H), 2.72 (dd, J=17.5, 11.5 Hz, 1H), 2.38 (dd, J=16.5, 2.0 Hz, 1H), 2.35-2.26 (m, 2H), 2.25-2.13 (m, 3H), 2.03-1.88 (m, 4H), 1.85 (d, J=5.0 Hz, 1H), 1.85-1.80 (m, 1H), 1.75-1.56 (m, 5H), 1.31-1.02 (m, 7H), 0.83 (d, J=6.5 Hz, 3H).

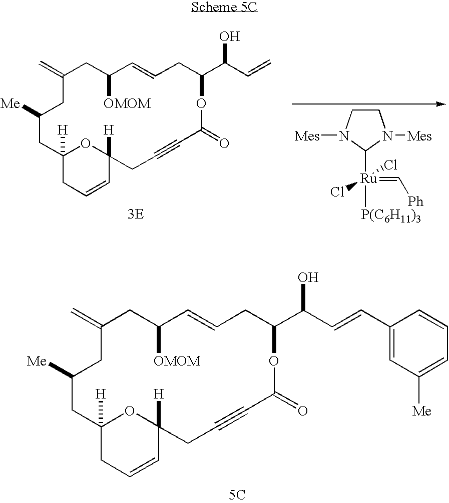

A solution of macrolactone 3E (8 mg, 18 μmol) in dichloromethane (3 mL) was degassed using the freeze-pump-thaw method. To the resulting solution was added 3-methylstyrene (120 μL, 0.9 mmol) followed by Grubbs' 2nd generation metathesis catalyst (3 mg, 3.6 μmol). The resulting solution was stirred at room temperature for 16 h, then additional 3-methylstyrene (47 μmol, 0.36 mmol) and catalyst (1.5 mg, 1.5 μmol) were added. After an additional 6 h of stirring at room temperature, a final addition of 3-methylstyrene (47 μL, 0.36 mmol) and catalyst (1.5 mg, 1.5 μmol) was added. The resulting solution was stirred overnight (18 h). The solvent was then removed in vacuo, and the residue was purified by flash chromatography (silica gel, 1:3 EtOAc:pentane) to provide macrolactone 5C (3.8 mg, 40%) as an amorphous solid. The product displayed only one observable spot by TLC at the reported R_{f} and in the solvent system used for chromatographic purification. ¹H NMR: (600 MHz, CDCl₃) δ 7.23-7.16 (m, 3H), 7.08 (d, J=6.6 Hz, 1H), 6.67 (d, J=15.6 Hz, 1H), 6.15 (dd, J=15.6, 6.6 Hz, 1H), 5.94-5.90 (m, 1H), 5.70-5.64 (m, 1H), 5.64-5.60 (m, 1H), 5.55 (dd, J=15.6, 7.2 Hz, 1H), 5.13-5.09 (m, 1H), 4.87 (s, 1H), 4.81 (s, 1H), 4.65 (d, J=7.2 Hz, 1H), 4.48 (d, J=6.6 Hz, 1H), 4.47-4.42 (m, 1H), 4.36-4.31 (m, 1H), 4.26-4.22 (m, 1H), 3.73-3.67 (m, 1H), 3.31 (s, 3H), 2.72 (dd, J=18.0, 11.4 Hz, 1H), 2.42-2.36 (m, 3H), 2.35 (s, 3H), 2.25-2.19 (m, 2H), 2.19-2.14 (m, 1H), 2.02 (d, J=5.4 Hz, 1H), 2.01-1.95 (m, 2H), 1.95-1.89 (m, 1H), 1.85 (dd, J=13.2, 9.6 Hz, 1H), 1.63-1.57 (m, 1H), 1.13-1.07 (m, 1H), 0.84 (d, J=6.0 Hz, 1H).

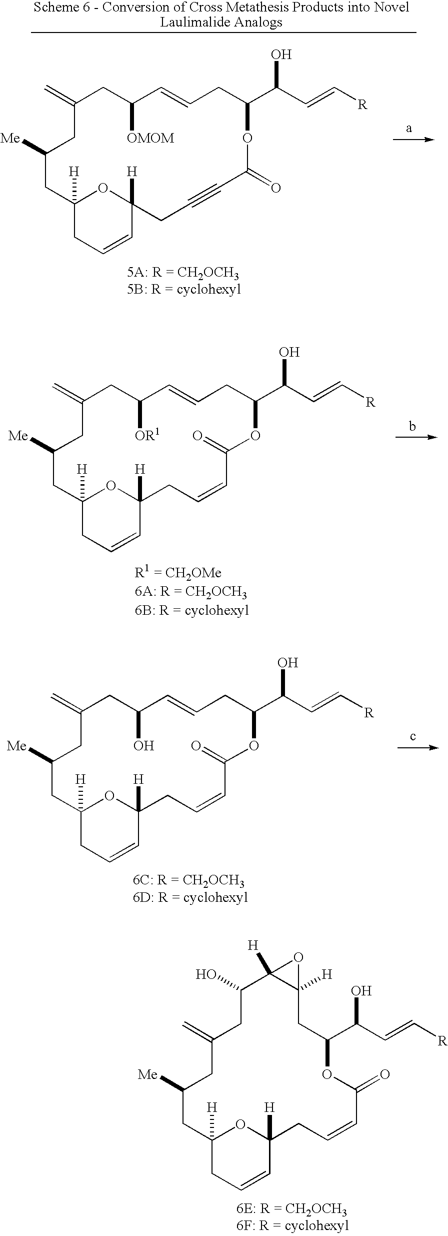

Step a: To a solution of macrolactone 5A (1.4 mg, 2.9 μmol) in ethyl acetate (0.5 mL) and 1-hexene (0.5 mL) was added Lindlar catalyst (1.2 mg, 0.6 μmol Pd) and quinoline (0.3 μL, 2.9 μmol). To this homogeneous mixture was applied a balloon of H₂. The resulting mixture was stirred at room temperature for 45 minutes, then filtered through a pad of Celite, eluting with EtOAc (3×5 mL). The organic layer was washed with 1N HCl (2 mL), saturated aqueous NaHCO₃ (2 mL), water (2 mL), and brine (2 mL), then dried (MgSO₄), filtered, and concentrated in vacuo. The residue was purified by flash chromatography (silica gel, 1:2 EtOAc:pentane) to provide enoate 6A (1.3 mg, 93%) as a colorless oil. The product displayed only one observable spot by TLC at the reported R_{f} and in the solvent system used for chromatographic purification. ¹H NMR: (500 MHz, CDCl₃) δ 6.41-6.34 (m, 1H), 5.93-5.81 (m, 3H), 5.76-5.69 (m, 2H), 5.64-5.57 (m, 1H), 5.46 (dd, J=15.5, 7.5 Hz, 1H), 4.99-4.94 (m, 1H), 4.84 (s, 1H), 4.78 (s, 1H), 4.66 (d, J=7.0 Hz, 1H), 4.48 (d, J=6.5 Hz, 1H), 4.25-4.11 (m, 3H), 3.94 (d, J=5.5 Hz, 2H), 3.87-3.80 (m, 1H), 3.65-3.56 (m, 1H), 3.323 (s, 3H), 3.319 (s, 3H), 2.42-2.33 (m, 2H), 2.32-2.25 (m, 2H), 2.22-2.11 (m, 3H), 2.00 (d, J=6.0 Hz, 1H), 1.90-1.82 (m, 1H), 1.78-1.67 (m, 2H), 1.66-1.59 (m, 1H), 1.17-1.10 (m, 1H), 0.85 (d, J=6.0 Hz, 3H).

Compound 6B was prepared by the method used above to prepare 6A by substituting alkyne 5B for alkyne 5A. Compound 6B ¹H NMR: (500 MHz, CDCl₃) δ 6.39-6.32 (m, 1H), 5.92-5.88 (m, 1H), 5.87-5.82 (m, 1H), 5.74-5.66 (m, 2H), 5.64-5.57 (m, 1H), 5.44 (dd, J=16.0, 8.5 Hz, 1H), 5.38 (ddd, J=16.0, 7.5, 2.0 Hz, 1H), 4.96-4.90 (m, 1H), 4.84 (s, 1H), 4.78 (s, 1H), 4.67 (d, J=6.0 Hz, 1H), 4.48 (d, J=6.0 Hz, 1H), 4.26-4.20 (m, 1H), 4.18-4.12 (m, 1H), 4.12-4.06 (m, 1H), 3.87-3.81 (m, 1H), 3.63-3.55 (m, 1H), 3.32 (s, 3H), 2.37-2.25 (m, 4H), 2.23-2.11 (m, 3H), 2.01-1.93 (m, 1H), 1.89 (d, J=6.0 Hz, 1H), 1.89-1.82 (m, 1H), 1.78-1.61 (m, 7H), 1.31-1.00 (m, 7H), 0.86 (d, J=6.5 Hz, 3H).

Step b: To a solution of MOM ether 6A (1.3 mg, 2.7 μmol) in CH₂Cl₂ (150 μL) was added triethylamine (0.75 μL, 5.4 μmol). The resulting solution was cooled to −78° C. and dimethylbromoborane (22 μL of a 0.6 M solution in DCE, 13.2 μmol) was added over 5 minutes down the side of the flask. When the addition was complete, the reaction flask was removed briefly from the cold bath and shaken in order to mix the reagents. The resulting solution was stirred at −78° C. for 90 minutes, and then was quenched by simultaneous addition of THF (0.5 mL) and saturated aqueous NaHCO₃ (0.3 mL). The mixture was removed from the cold bath and allowed to warm to room temperature over 1 h, then diluted with diethyl ether (10 mL). The organic layer was washed with water (3 mL) and brine (3 mL), then dried (Na₂SO₄), filtered, and concentrated in vacuo. The residue was purified by flash chromatography (silica gel, 1:2 EtOAc:pentane) to provide allylic alcohol 6C (0.9 mg, 75%) as a clear oil. The product displayed only one observable spot by TLC at the reported R_{f} and in the solvent system used for chromatographic purification. ¹H NMR: (500 MHz, CDCl₃) δ 6.39-6.32 (m, 1H), 5.94-5.80 (m, 3H), 5.76-5.69 (m, 2H), 5.64-5.58 (m, 2H), 5.02-4.96 (m, 1H), 4.86 (bs, 2H), 4.22-4.12 (m, 3H), 3.94 (d, J=5.5 Hz, 2H), 3.90-3.84 (m, 1H), 3.59-3.51 (m, 1H), 3.32 (s, 3H), 2.38-2.34 (m, 2H), 2.34-2.19 (m, 3H), 2.18-2.08 (m, 2H), 1.97 (d, J=5.0 Hz, 1H), 1.87-1.71 (m, 4H), 1.68-1.62 (m, 1H), 1.13 (ddd, J=14.5, 8.0, 4.0 Hz, 1H), 0.86 (d, J=6.0 Hz, 3H).

Compound 6D was prepared by the method used above to prepare 6C by substituting MOM ether 6B for MOM ether 6A. Compound 6D ¹H NMR: (600 MHz, CDCl₃) δ 6.38-6.32 (m, 1H), 5.93-5.90 (m, 1H), 5.86-5.80 (m, 1H), 5.75-5.68 (m, 2H), 5.64-5.60 (m, 2H), 5.39 (ddd, J=15.6, 6.6, 1.2 Hz, 1H), 4.97-4.93 (m, 1H), 4.86 (br s, 2H), 4.21-4.13 (m, 2H), 4.11-4.07 (m, 1H), 3.90-3.85 (m, 1H), 3.58-3.51 (m, 1H), 2.36-2.31 (m, 3H), 2.31-2.19 (m, 3H), 2.19-2.10 (m, 3H), 2.01-1.94 (m, 1H), 1.87-1.62 (m, 8H), 1.33-1.21 (m, 3H), 1.19-1.10 (m, 2H), 1.10-1.02 (m, 2H), 0.87 (d, J=6.0 Hz, 3H).

Step c: Diol 6C (3.0 mg, 6.72 μmol) was azeotropically dried with benzene (3×2 mL). The residue was then dissolved in dichloromethane (0.7 mL). To this stirred solution under positive pressure of N₂ at room temperature was added 4 Å molecular sieves (80 mg) in one portion. The resulting suspension was stirred at room temperature for 30 min. A solution of (+)-diisopropyl tartrate (freshly distilled, 85° C. at 0.02 torr, 37 μL of a 0.25 M stock solution in CH₂Cl₂, 9.20 μmol) was then added dropwise over 1 min. The reaction mixture was cooled to −30° C. and a solution of Ti(Oi-Pr)₄ (freshly distilled, 34 μL of a 0.18 M stock solution in CH₂Cl₂, 6.18 μmol) was added dropwise over 1 min. The reaction was then warmed to −20° C. over 15 min and stirred for an additional 45 minutes. The reaction mixture was cooled back down to −40° C. and t-butyl hydroperoxide (5-6 M in decane, pre-dried with 3 Å molecular sieves for 30 min, 3.7 μL, 20.2 μmol) was added. The mixture was allowed to warm to −20° C. over 30 minutes, then placed in a −20° C. freezer and left overnight. After 20 h, the reaction was removed from the freezer and placed in an ice/water bath (0° C.). To the reaction mixture was added 3M NaOH (0.3 mL) and brine (0.4 mL) simultaneously. The resulting mixture was stirred vigorously at 0° C. for 1 h. The suspension was extracted with CH₂Cl₂ (3×5 mL), and the combined organic fractions were washed with water (5 mL) and brine (5 mL), then dried (Na₂SO₄), filtered, and concentrated in vacuo. The residue was purified by flash chromatography (silica gel, 2:1 EtOAc:pentane) to provide analogue 6E (1.7 mg, 55%) as a colorless oil. The product displayed only one observable spot by TLC at the reported R_{f}. ¹H NMR: (600 MHz, CDCl₃) δ 6.48-6.42 (m, 1H), 5.93-5.81 (m, 3H), 5.75-5.68 (m, 2H), 5.15 (ddd, J=10.8, 4.8, 1.2 Hz, 1H), 4.86 (s, 1H), 4.85 (s, 1H), 4.33-4.28 (m, 1H), 4.23-4.19 (m, 1H), 4.09-4.04 (m, 1H), 3.93 (d, J=6.0 Hz, 2H), 3.78-3.67 (m, 2H), 3.33 (s, 3H), 3.08-3.04 (m, 1H), 2.89 (t, J=3.0 Hz, 1H), 2.40-2.34 (m, 2H), 2.26-2.20 (m, 1H), 2.15-2.10 (m, 1H), 2.06-1.97 (m, 2H), 1.96-1.88 (m, 2H), 1.81-1.75 (m, 1H), 1.74-1.68 (m, 1H), 1.54-1.47 (m, 1H), 1.45 (dd, J=14.4, 7.2 Hz, 1H), 1.36-1.27 (m, 2H), 0.83 (d, J=6.6 Hz, 3H).

Diol 6D (7.4 mg, 15.3 μmol) was azeotropically dried with benzene (3×2 mL). The residue was then dissolved in CH₂Cl₂ (1.5 mL). To this stirred solution under positive pressure of N₂ at room temperature was added 4 Å molecular sieves (185 mg) in one portion. The resulting suspension was stirred at room temperature for 30 min. A solution of (+)-diisopropyl tartrate (freshly distilled, 85° C. at 0.02 torr, 0.25 M stock solution in CH₂Cl₂, 69 μL, 17.3 μmol) was then added dropwise over 1 min. The reaction mixture was cooled to −30° C. and a solution of Ti(Oi-Pr)₄ (freshly distilled, 0.18 M stock solution in CH₂Cl₂, 64 μL, 11.5 μmol) was added dropwise over 1 min. The reaction was then warmed to −20° C. over 15 min and stirred for an additional 45 minutes. The reaction mixture was cooled back down to −40° C. and t-butyl hydroperoxide (5-6 M in decane, pre-dried with 3 Å molecular sieves for 30 min, 8.3 μL, 45.9 μmol) was added. The mixture was allowed to warm to −20° C. over 30 minutes, then placed in a −20° C. freezer. After 6 h, the reaction was removed from the freezer and placed in an ice-water bath (0° C.). To the reaction mixture was added 3M NaOH aq. (1.0 mL) and brine (1.2 mL) simultaneously. The resulting mixture was stirred vigorously at 0° C. for 1 h. The suspension was extracted with CH₂Cl₂ (3×10 mL), and the combined organic fractions were washed with water (5 mL) and brine (5 mL), then dried (Na₂SO₄), filtered, and concentrated in vacuo. The residue was purified via flash chromatography (silica gel, 1:2 EtOAc:pentane) to provide 6F (3.9 mg, 51%) and the product of double epoxidation (1.5 mg, 19%) as colorless oils. Each product displayed only one observable spot by TLC at the reported R_{f} and in the solvent system used for chromatographic purification. ¹H NMR data for 6F: (500 MHz, CDCl₃) δ 6.47-6.40 (m, 1H), 5.93-5.88 (m, 1H), 5.86-5.81 (m, 1H), 5.72-5.66 (m, 2H), 5.38 (ddd, J=15.5, 6.0, 1.0 Hz, 1H), 5.12 (ddd, J=11.0, 5.5, 1.8 Hz, 1H), 4.85 (s, 1H), 4.84 (s, 1H), 4.33-4.28 (m, 1H), 4.13-4.05 (m, 2H), 3.79-3.68 (m, 2H), 3.08-3.04 (m, 1H), 2.90-2.88 (m, 1H), 2.40-2.32 (m, 2H), 2.26-2.18 (m, 1H), 2.15-2.08 (m, 1H), 2.06-1.87 (m, 6H), 1.81-1.64 (m, 7H), 1.50-1.41 (m, 2H), 1.35-1.29 (m, 1H), 1.29-1.20 (m, 2H), 1.18-1.11 (m, 1H), 1.10-0.99 (m, 2H), 0.82 (d, J=6.5 Hz, 3H).

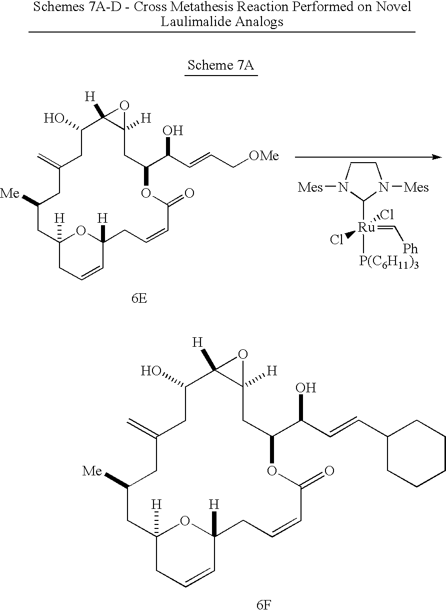

A solution of laulimalide analog 6E (1.3 mg, 2.8 μmol) in dichloromethane (0.8 mL) was degassed using the freeze-pump-thaw method. To this solution was added vinylcyclohexane (19 μL, 0.14 mmol) followed by Grubbs' 2nd generation metathesis catalyst (0.2 mg, 0.28 μmol). The resulting solution was stirred at room temperature for 20 h, after which time the solvent was evaporated under a stream of N₂. The residue was purified by flash chromatography (silica gel, EtOAc:pentane 1:1) to provide cyclohexane side chain analog 6F (0.7 mg, 50%) as a clear oil. Physical and spectral data of the product were identical to the values reported above.

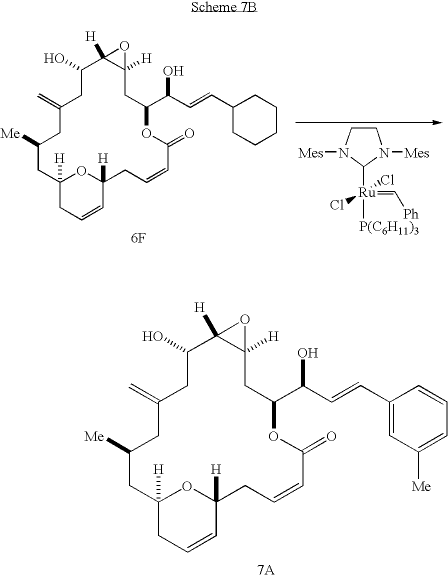

A solution of cyclohexane side chain analogue 6F (2.0 mg, 4 μmol) in dichloromethane (1 mL) was degassed using the freeze-pump-thaw method. To this solution was added 3-methylstyrene (27 μL, 0.2 mmol) followed by the Grubbs 2nd generation metathesis catalyst (0.7 mg, 0.8 μmol). The resulting pink solution was allowed to stir at room temperature for 18 h. The solvent was evaporated under a stream of N₂, and the residue was purified using flash chromatography (silica gel, EtOAc:pentane 1:2) to provide aryl side chain analogue 7A (1.0 mg, 50%) as a clear oil. The product displayed only one observable spot by TLC at the reported R_{f} and in the solvent system used for chromatographic purification. ¹H NMR: (600 MHz, CDCl₃) δ 7.23-7.14 (m, 3H), 7.10-7.06 (m, 1H), 6.64 (d, J=16.2 Hz, 1H), 6.46-6.40 (m, 1H), 6.15 (dd, J=16.2, 6.0 Hz, 1H), 5.94-5.90 (m, 1H), 5.86-5.82 (m, 1H), 5.71-5.66 (m, 1H), 5.26-5.22 (m, 1H), 4.86 (s, 1H), 4.85 (s, 1H), 4.38-4.34 (m, 1H), 4.32-4.27 (m, 1H), 4.11-4.06 (m, 1H), 3.79-3.69 (m, 2H), 3.11-3.07 (m, 1H), 2.92-2.90 (m, 1H), 2.47-2.36 (m, 1H), 2.34 (s, 3H), 2.29-2.10 (m, 3H), 2.07-1.97 (m, 3H), 1.96-1.88 (m, 2H), 1.82-1.75 (m, 1H), 1.75-1.68 (m, 1H), 1.49-1.41 (m, 1H), 1.37-1.26 (m, 2H), 0.83 (d, J=6.6 Hz, 3H).

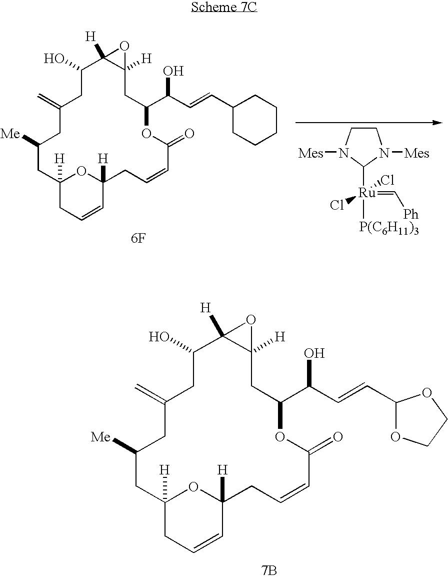

A solution of cyclohexane side chain analogue 6F (2 mg, 4 μmol) in dichloromethane (1 mL) was degassed using the freeze-pump-thaw method. To this solution at room temperature was added 2-vinyl-1,3-dioxolane (placed on vacuum pump overnight before use to remove trace acrolein, 8 μL, 0.08 mmol) followed by the Grubbs 2nd generation metathesis catalyst (1 mg, 12 μmol). The resulting solution was allowed to stir at room temperature for 4 h. The solvent was removed under a stream of N₂ and the residue was purified using flash chromatography (silica gel, EtOAc:pentane 2:1) to provide dioxolane side chain analogue 7B (0.8 mg, 41%) as a colorless oil. The product displayed only one observable spot by TLC at the reported R_{f} and in the solvent system used for chromatographic purification. ¹H NMR: (600 MHz, CDCl₃) δ 6.48-6.42 (m, 1H), 5.94 (dd, J=15.0, 5.4 Hz, 1H), 5.92-5.88 (m, 1H), 5.86-5.79 (m, 2H), 5.72-5.68 (m, 1H), 5.29 (d, J=5.7 Hz, 1H), 5.20-5.16 (m, 1H), 4.86 (s, 1H), 4.85 (s, 1H), 4.33-4.28 (m, 1H), 4.28-4.25 (m, 1H), 4.09-4.05 (m, 1H), 4.01-3.94 (m, 2H), 3.93-3.86 (m, 2H), 3.78-3.67 (m, 2H), 3.08-3.04 (m, 1H), 2.90-2.88 (m, 1H), 2.39-2.33 (m, 2H), 2.26-2.20 (m, 1H), 2.14-2.09 (m, 1H), 2.06-1.98 (m, 2H), 1.97 (d, J=6.6 Hz, 1H), 1.95-1.92 (m, 1H), 1.91-1.89 (m, 1H), 1.81-1.75 (m, 1H), 1.74-1.67 (m, 1H), 1.54-1.48 (m, 1H), 1.48-1.42 (m, 1H), 1.35-1.30 (m, 1H), 0.83 (d, J=6.6 Hz, 3H).

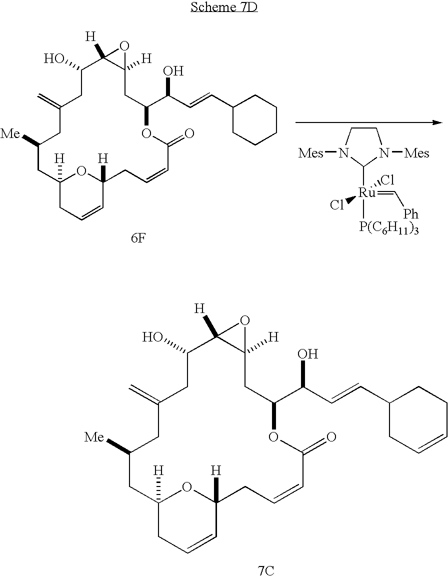

A solution of cyclohexane side chain analogue 6F (2 mg, 4 μmol) in dichloromethane (1 mL) was degassed using the freeze-pump-thaw method. To the resulting solution at room temperature was added vinylcyclohexene (racemic, 26 μL, 0.2 mmol) followed by the Grubbs 2nd generation metathesis catalyst (1 mg, 1.2 μmol). The resulting solution was allowed to stir at room temperature for 18 h. The solvent was removed under a stream of N₂ and the residue was purified by flash chromatography (silica gel, EtOAc:pentane 1:2) to provide cyclohex-3-en-1-yl side chain analogue 7C (1.1 mg, 55%, 1:1 mixture of diastereomers) as a colorless oil. The product displayed only one observable spot by TLC at the reported R_{f} and in the solvent system used for chromatographic purification. ¹H NMR: (600 MHz, CDCl₃) δ 6.47-6.41 (m, 1H), 5.93-5.89 (m, 1H), 5.86-5.82 (m, 1H), 5.77 (dd, J=15.6, 6.6 Hz, 1H), 5.72-5.68 (m, 1H), 5.68-5.63 (m, 2H), 5.45 (dd, J=15.6, 6.6 Hz, 1H), 5.13 (ddd, J=11.4, 5.4, 1.8 Hz, 1H), 4.86 (s, 1H), 4.85 (s, 1H), 4.33-4.28 (m, 1H), 4.15-4.11 (m, 1H), 4.09-4.04 (m, 1H), 3.79-3.68 (m, 2H), 3.09-3.05 (m, 1H), 2.91-2.89 (m, 1H), 2.40-2.32 (m, 2H), 2.32-2.25 (m, 1H), 2.25-2.19 (m, 1H), 2.15-1.97 (m, 6H), 1.95-1.87 (m, 2H), 1.86-1.67 (m, 5H), 1.52-1.42 (m, 2H), 1.42-1.30 (m, 2H), 0.83 (d, J=6.6 Hz, 3H).

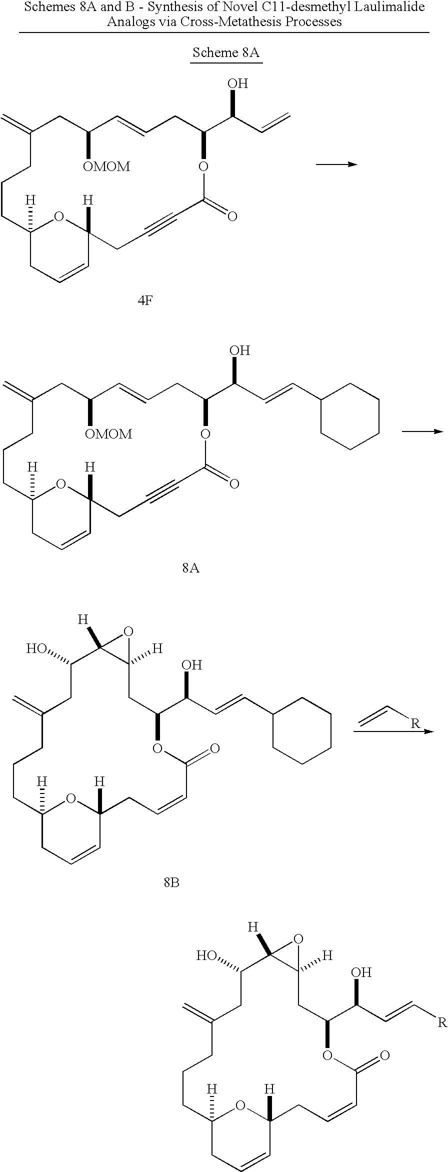

Novel C11-desmethyl laulimalide analogs are prepared by transforming 4F into 8B following the procedure described for the transformation of 3E to 5B to 6F. Compound 8B is used to prepare novel analogs following the procedure described for described in Schemes 7B-D.

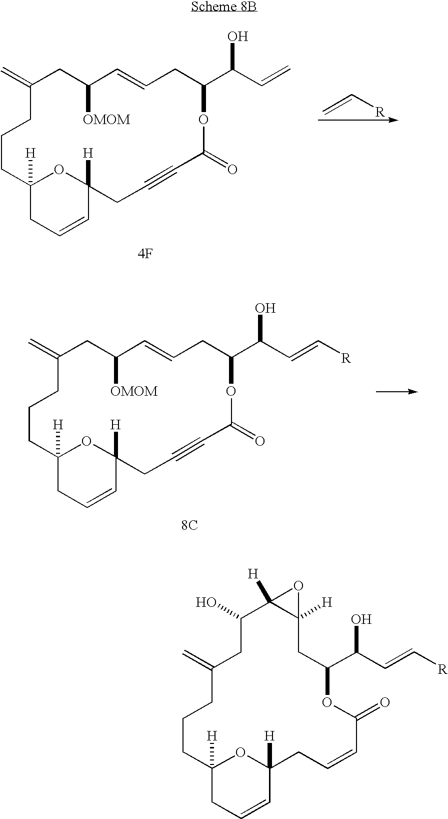

In further embodiments, novel C11-desmethyl laulimalide analogs are prepared by transforming vinyl macrolactone 4F into intermediate 8C following the procedure described for schemes 5A-C. Subsequent processing of intermediate 8C into novel C11-desmethyl laulimalide analogs proceeds through the methods described in Scheme 6.

### II Biological Screening

Laulimalide analogs disclosed herein were evaluated for the ability to inhibit the proliferation of human tumor cells using the sulforhodamine assay format (Mooberry et al., Cancer Research, 1999, 59, 653-660). The two cells lines selected were the human breast carcinoma MDA-MB-435 and the corresponding MDR cell line NCI/ADR, which overexpresses the PGP efflux pump protein. Results are shown in the table below. Compound 7B was demonstrated to be chemically stable under the conditions of the assay.

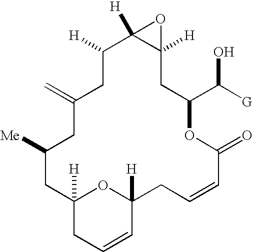

| | | | NCI/ | |
|---|---|---|---|---|
| | | MDA-MB- | ADR | |
| Com- | | 435 | IC₅₀ | Fold |
| pound | G group | IC₅₀ (nM) | (nM) | Resistance |
| Laulima-lide | 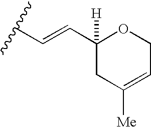 | 5.7 ± 0.6 | n/a | n/a |
| 6E | 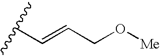 | 7900 ± 320 | 22900 ±90 | 2.9 |
| 6F | 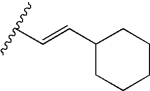 | 1170 ± 91 | 1210 ±99 | 1.0 |
| 6G | 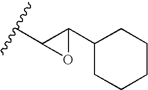 | 1350 ± 50 | 2380 ±90 | 1.8 |
| 7A | 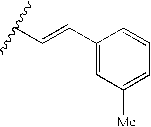 | 7300 ± 1270 | 12400 ±1400 | 1.7 |
| 7B | 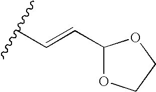 | >50000 | n/a | n/a |
| 7C | 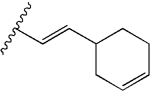 | 368 ± 80 | n/a | n/a |

n/a = not available

While certain embodiments of the present disclosure have been shown and described herein, such embodiments are provided by way of example only. It should be understood that various alternatives to the embodiments described herein may be employed in practicing the present disclosure. It is intended that the following claims define the scope of what is presented herein and that methods and structures within the scope of these claims and their equivalents be covered thereby.

I claim:

1. A compound of Formula (V) or pharmaceutically acceptable solvate, pharmaceutically acceptable salt, or pharmaceutically acceptable prodrug thereof: 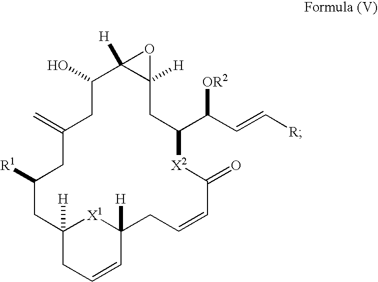
wherein:
R is selected from: 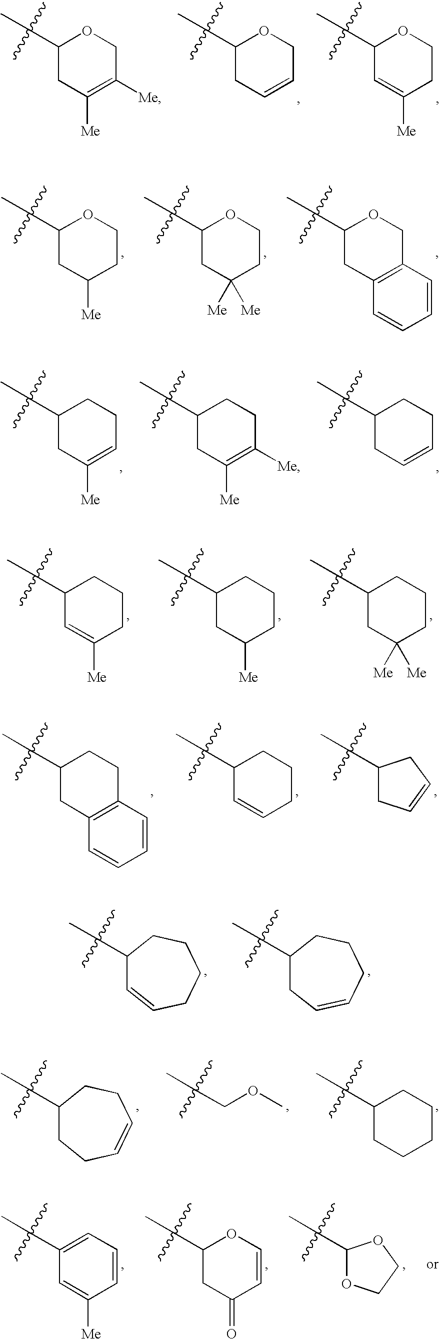 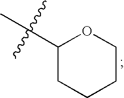
R¹ is H or methyl;
R² is H, methyl or COCH₃;
X¹ is O, NH or N-methyl; and
X² is O, NH or N-methyl.

2. The compound of claim 1, wherein R¹ is methyl.

3. The compound of claim 1, wherein R¹ is H.

4. The compound of claims 2 or 3 wherein X¹ is O and X² is O.

5. The compound of claim 4, wherein R² is H.

6. The compound of claim 5 selected from:

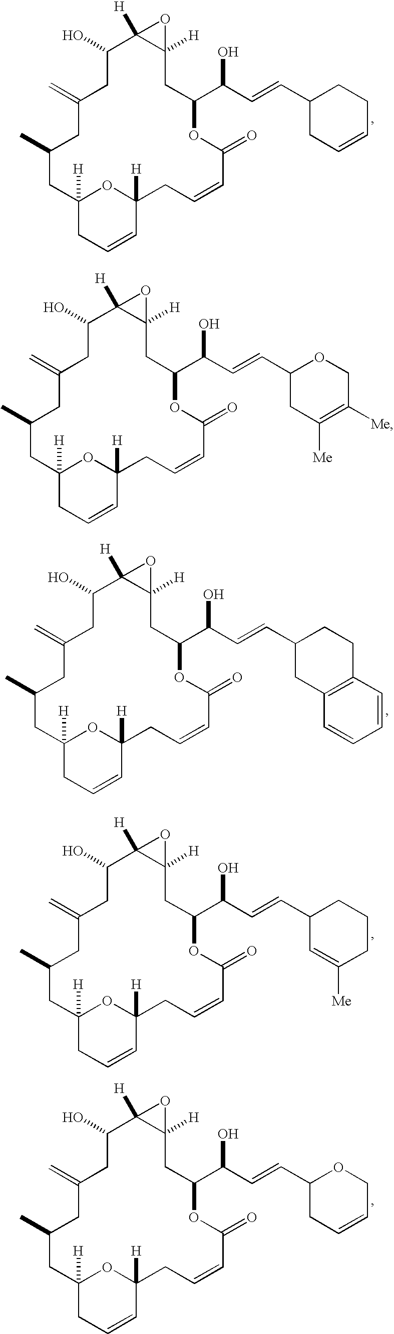

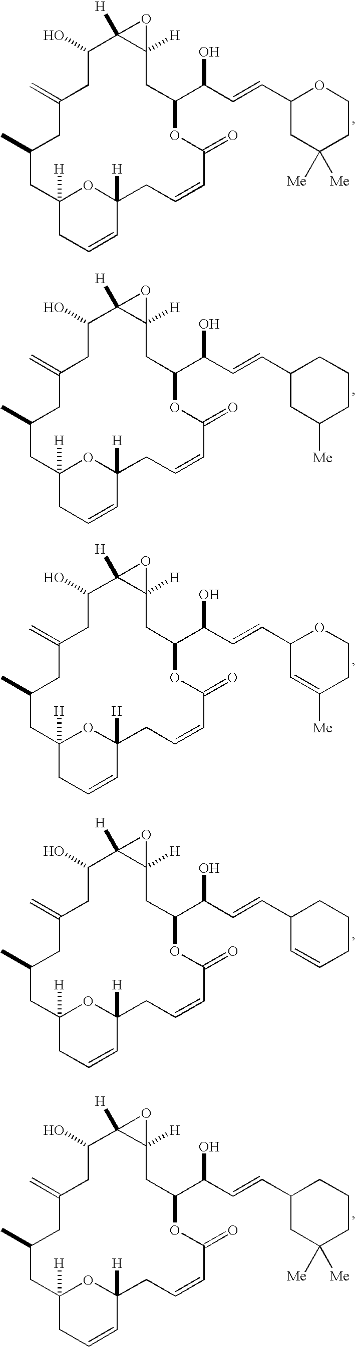

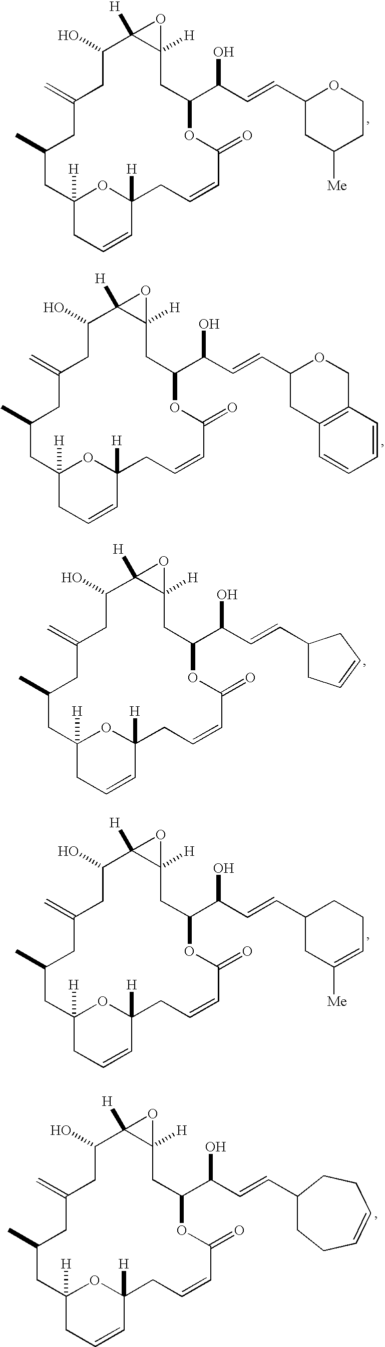

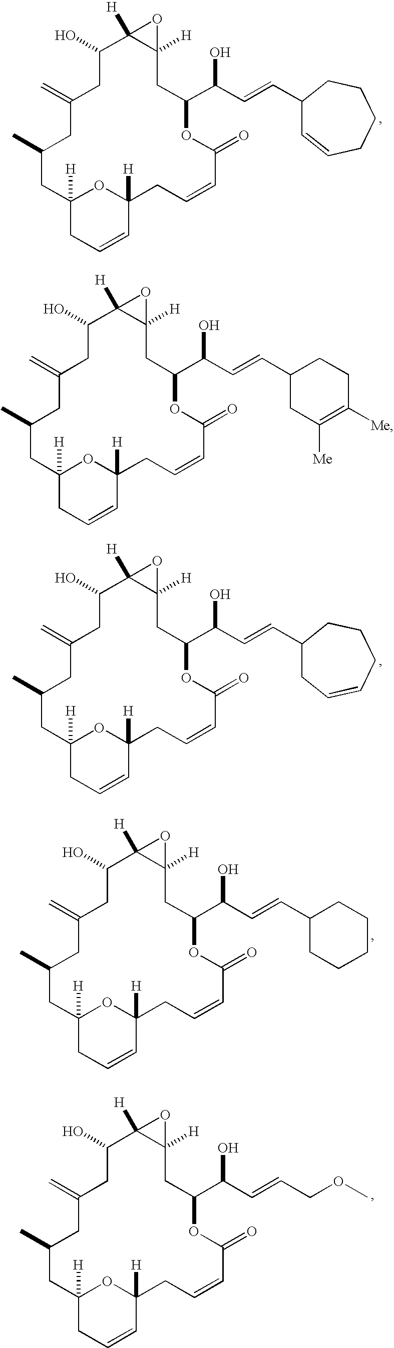

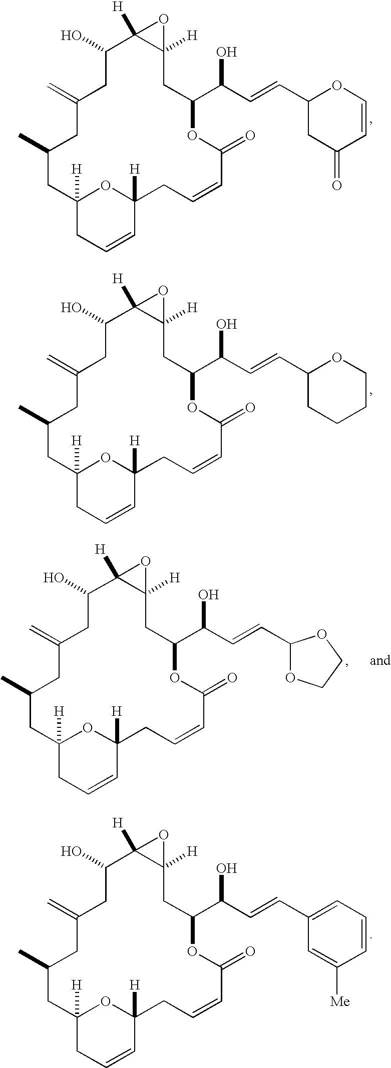

7. The compound of claim 5 selected from:

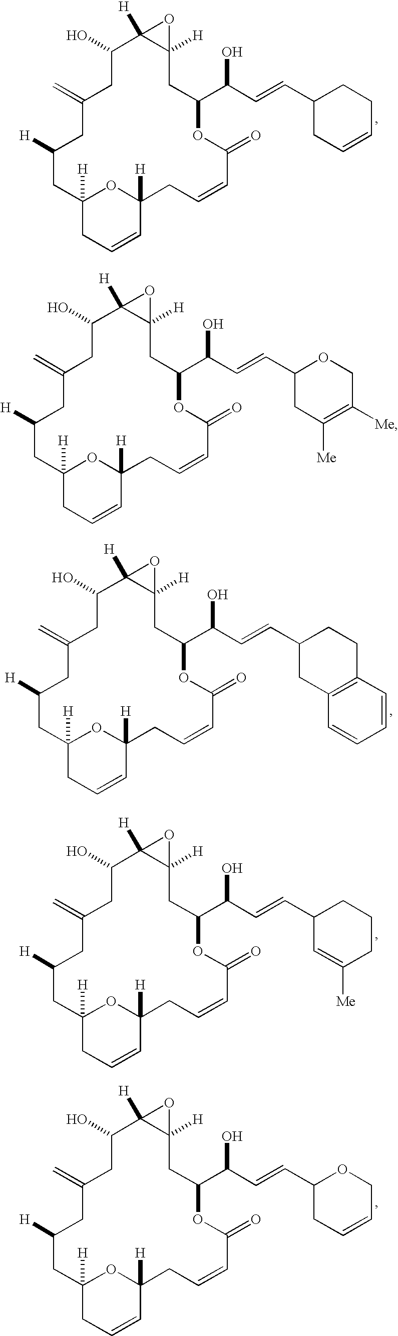

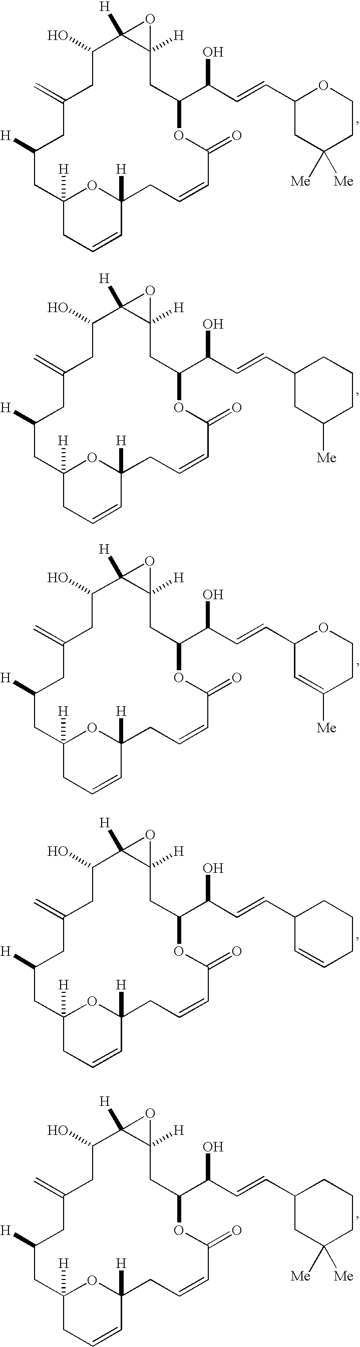

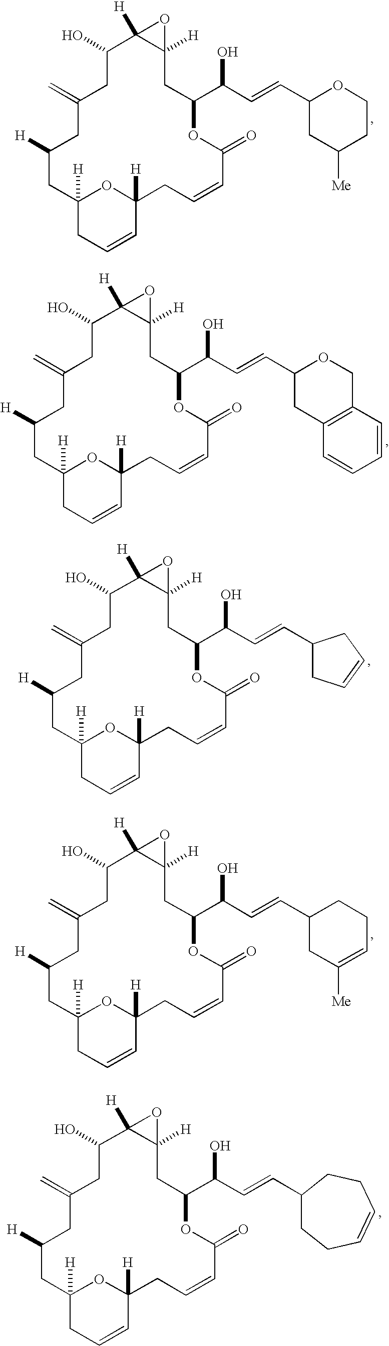

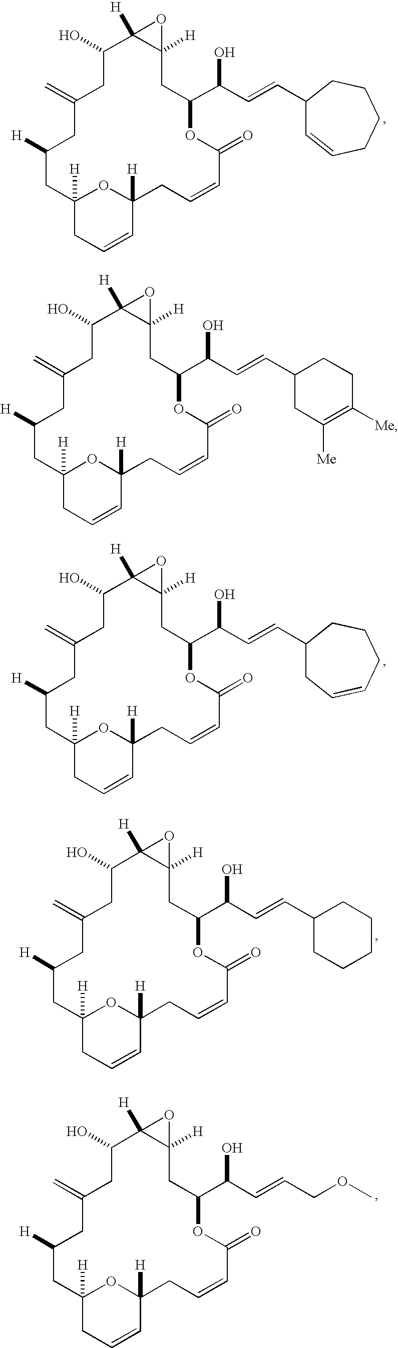

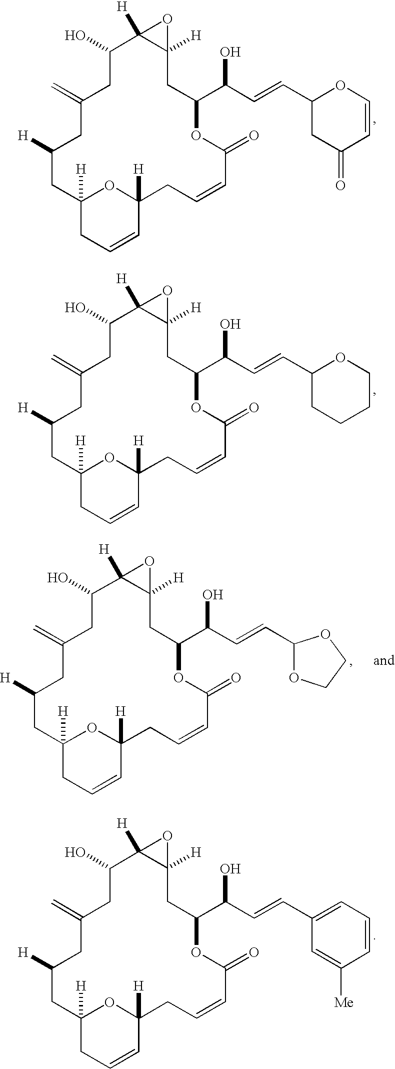

8. A pharmaceutical composition comprising a compound of Formula (V) or a pharmaceutically acceptable solvate, pharmaceutically acceptable salt, or pharmaceutically acceptable prodrug; and a pharmaceutically acceptable carrier or excipient.

9. A process for the preparation of a compound of Formula (I) comprising:
a. presenting a compound of Formula (II): 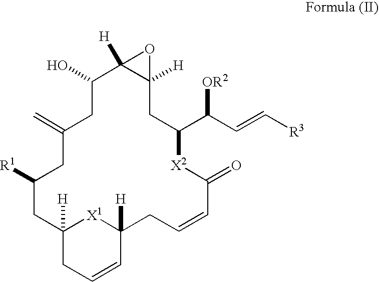
in a reactor; and
b. subjecting the compound of Formula (II) to a cross-metathesis reaction with a cross-metathesis reactive alkene; the cross-metathesis reaction being promoted by a cross-metathesis catalyst, to produce the compound of Formula (I): 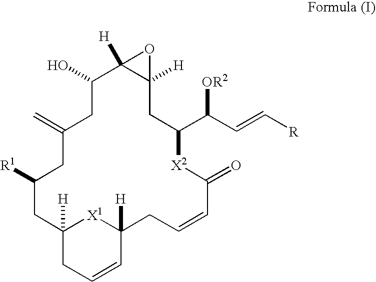
wherein:
R is an optionally substituted C₁-C₁₀ alkyl, an optionally substituted C₂-C₁₀ alkenyl, an optionally substituted C₃-C₈ cycloalkyl, an optionally substituted C₄-C₈ cycloalkenyl, an optionally substituted aryl, an optionally substituted heterocycloalkyl, an optionally substituted heterocycloalkenyl, an optionally substituted heteroaryl;
R¹ is H or methyl;
R² is H, methyl or COCH₃;
R³ is H, methyl, ethyl, propyl, butyl, pentyl, cyclohexyl, isopropyl or methoxymethyl;
X¹ is O, NH or N-alkyl; and
X² is O, NH or N-alkyl.

10. The process of claim 9, wherein the cross-metathesis reactive alkene is CH₂═CH—Y, where Y is selected from the group:

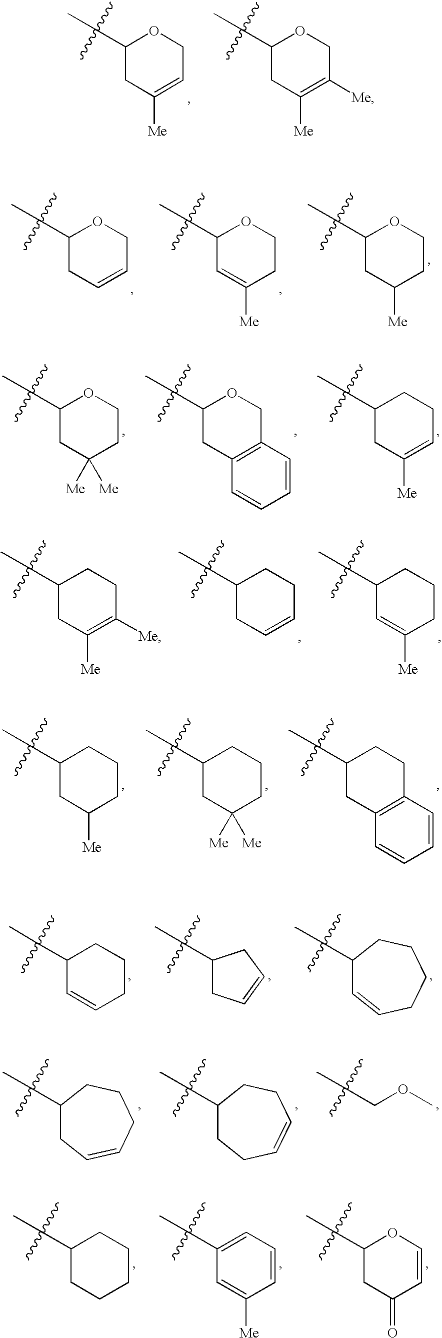

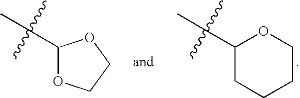

11. The process of claim 9, wherein the cross-metathesis catalyst is selected from a catalyst containing Ni, W, Ru or Mo metal.

12. The process of claim 11, wherein the cross-metathesis catalyst contains Ru.

13. The process of claim 12, wherein the cross-metathesis catalyst is selected from:
dichloro(phenylmethylene)bis(tricyclohexylphosphine)ruthenium(II),
[1,3-bis-(2,4,6-trimethylphenyl)-2-imidazolidinylidene]dichloro(phenylmethylene)(tricyclohexylphosphine)ruthenium(II),
dichloro[[2-(1-methylethoxy)phenyl]methylene](tricyclohexylphosphine)ruthenium(II),
[1,3-bis(2,4,6-trimethylphenyl)-2-imidazolidinylidene]dichloro[[2-(1-methylethoxy)phenyl]methylene]ruthenium(II),
[1,3-bis(2-methylphenyl)-2-imidazolidinylidene]dichloro[[2-(1-methylethoxy)phenyl]methylene]ruthenium(II),
[1,3-bis(2,4,6-trimethylphenyl)-2-imidazolidinylidene]dichloro[3-(2-pyridinyl-κN)propylidene-κC]ruthenium (II),
[1,3-bis(2-methylphenyl)-2-imidazolidinylidene]dichloro(benzylidene)(tricyclohexylphosphine)ruthenium(II),
dichloro(3-methyl-2-butenylidene)bis(tricyclohexylphosphine)ruthenium(II),
[1,3-bis(2,4,6-trimethylphenyl)-2-imidazolidinylidene]dichloro(3-methyl-2-butenylidene)(tricyclohexylphosphine)ruthenium(II),
[1,3-bis(2,4,6-trimethylphenyl)-2-imidazolidinylidene]dichloro(benzylidene)bis(3-bromopyridine)ruthenium(II).

14. The process of claim 9, wherein the reaction is conducted at temperature of about 15° C. to about 35° C.

15. The process of claim 9, wherein the reaction is conducted at a temperature of about 20° C. to about 150° C. with thermal heating.

16. The process of claim 9, wherein the reaction is conducted at a temperature of about 20° C. to about 150° C. with microwave irradiation heating.

17. The process of claim 9, wherein the amount of cross-metathesis catalyst is between about 1 mol % and about 40 mol %.

18. A process for the preparation of a compound of Formula (I) comprising:
a. presenting a compound of Formula (III) 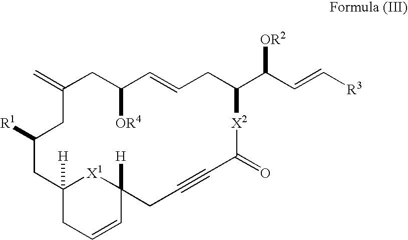
in a reactor;
b. subjecting the compound of Formula (III) to a cross-metathesis reaction with a cross-metathesis reactive alkene, said cross-metathesis reaction being promoted by a cross-metathesis catalyst; and
c. processing the product of the cross-metathesis reaction to obtain a compound of Formula (I); 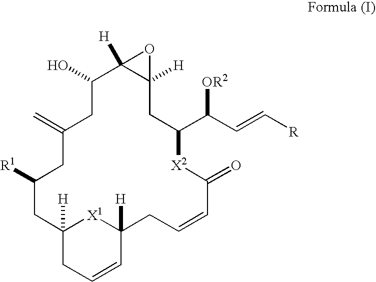
wherein:
R is an optionally substituted C₁-C₁₀ alkyl an optionally substituted C₂-C₁₀ alkenyl, an optionally substituted C₃-C₈ cycloalkyl, an optionally substituted C₄-C₈ cycloalkenyl, an optionally substituted aryl, an optionally substituted heterocycloalkyl, an optionally substituted heterocycloalkenyl, an optionally substituted heteroaryl;
R¹ is H or methyl;
R² is H, methyl or COCH₃;
R³ is H, methyl, ethyl, propyl, butyl, pentyl, cyclohexyl, isopropyl or methoxymethyl;
R⁴ is H or CH₂OCH₃;
X¹ is O, NH or N-alkyl; and
X² is O, NH or N-alkyl.

19. The process of claim 18, wherein the cross-metathesis reactive alkene is CH₂═CH—Y, where Y is selected from the group:

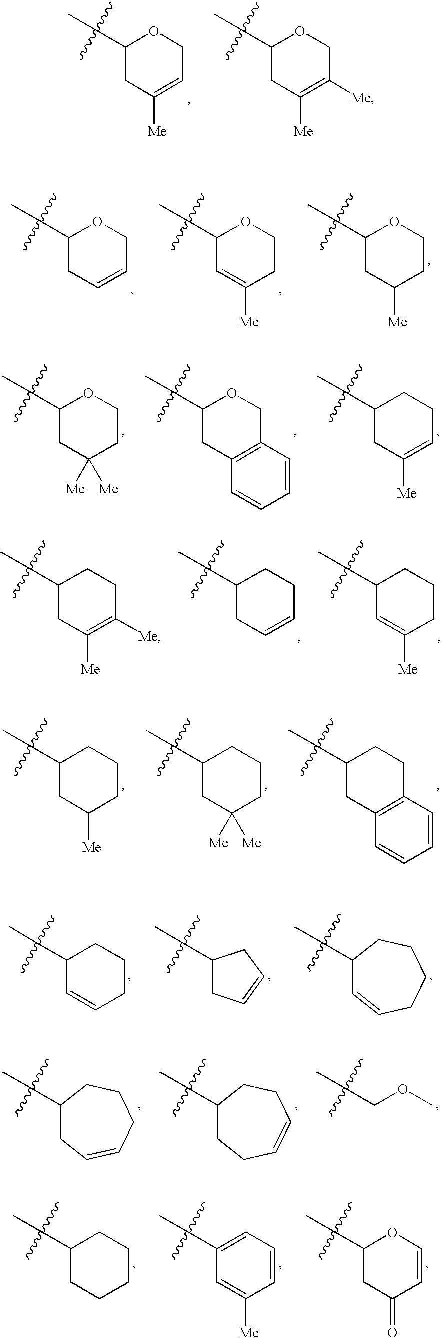

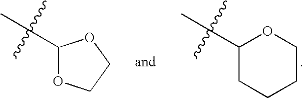

20. The process of claim 18, wherein the cross-metathesis catalyst is selected from a catalyst containing Ni, W, Ru or Mo metal.

21. The process of claim 19, wherein the cross-metathesis catalyst contains Ru.

22. The process of claim 21, wherein the cross-metathesis catalyst is selected from:
dichloro(phenylmethylene)bis(tricyclohexylphosphine)ruthenium(II),
[1,3-bis-(2,4,6-trimethylphenyl)-2-imidazolidinylidene]dichloro(phenylmethylene)(tricyclohexylphosphine)ruthenium(II),
dichloro[[2-(1-methylethoxy)phenyl]methylene](tricyclohexylphosphine)ruthenium(II),
[1,3-bis(2,4,6-trimethylphenyl)-2-imidazolidinylidene]dichloro[[2-(1-methylethoxy)phenyl]methylene]ruthenium(II),
[1,3-bis(2-methylphenyl)-2-imidazolidinylidene]dichloro[[2-(1-methylethoxy)phenyl]methylene]ruthenium(II),
[1,3-bis(2,4,6-trimethylphenyl)-2-imidazolidinylidene]dichloro[3-(2-pyridinyl-κN)propylidene-κC]ruthenium (II),
[1,3-bis(2-methylphenyl)-2-imidazolidinylidene]dichloro(benzylidene)(tricyclohexylphosphine)ruthenium (II),
dichloro(3-methyl-2-butenylidene)bis(tricyclohexylphosphine)ruthenium(II),
[1,3-bis(2,4,6-trimethylphenyl)-2-imidazolidinylidene]dichloro(3-methyl-2-butenylidene)(tricyclohexylphosphine)ruthenium(II),
[1,3-bis(2,4,6-trimethylphenyl)-2-imidazolidinylidene]dichloro(benzylidene)bis(3-bromopyridine)ruthenium(II).

23. The process of claim 18, wherein the reaction is conducted at temperature of about 15° C. to about 35° C.

24. The process of claim 18, wherein the reaction is conducted at a temperature of about 20° C. to about 150° C. with thermal heating.

25. The process of claim 18, wherein the reaction is conducted at a temperature of about 20° C. to about 150° C. with microwave irradiation heating.

26. The process of claim 18, wherein the amount of cross-metathesis catalyst is between about 1 mol % and about 40 mol %.

27. The process of claim 18 where R⁴ is CH₂OCH₃ and wherein processing the product comprises:
a. reducing an alkyne at C2-C3 to a cis-alkene by hydrogenation;
b. removing the methoxymethyl group of R⁴; and
c. epoxidizing an alkene at C16-C17.

28. The process of claim 18 where R⁴ is H, and wherein processing the product comprises:
a. reducing an alkyne at C2-C3 to a cis-alkene by hydrogenation; and
b. epoxidizing an alkene at C16-C17.

29. A process for the preparation of a compound of Formula (I) comprising:
a. presenting a compound of Formula (IV) 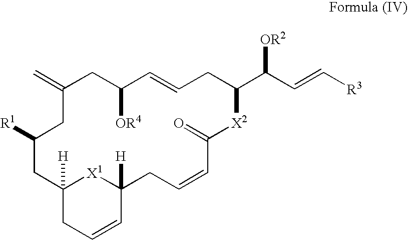
in a reactor;
b. subjecting the compound of Formula (IV) to a cross-metathesis reaction with a cross-metathesis reactive alkene, said cross-metathesis reaction being promoted by a cross-metathesis catalyst; and
c. processing the product of the cross-metathesis reaction to obtain a compound of Formula (I); 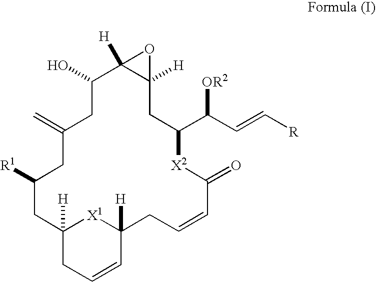
wherein:
R is an optionally substituted C₁-C₁₀ alkyl, an optionally substituted C₂-C₁₀ alkenyl, an optionally substituted C₃-C₈ cycloalkyl, an optionally substituted C₄-C₈ cycloalkenyl, an optionally substituted aryl, an optionally substituted heterocycloalkyl, an optionally substituted heterocycloalkenyl, an optionally substituted heteroaryl;
R¹ is H or methyl;
R² is H, methyl or COCH₃;
R³ is H, methyl, ethyl, propyl, butyl, pentyl, cyclohexyl, isopropyl or methoxymethyl;
R⁴ is H or CH₂OCH₃;
X¹ is O, NH or N-alkyl; and
X² is O, NH or N-alkyl.

30. The process of claim 29, wherein the cross-metathesis reactive alkene is CH₂═CH—Y, where Y is selected from the group:

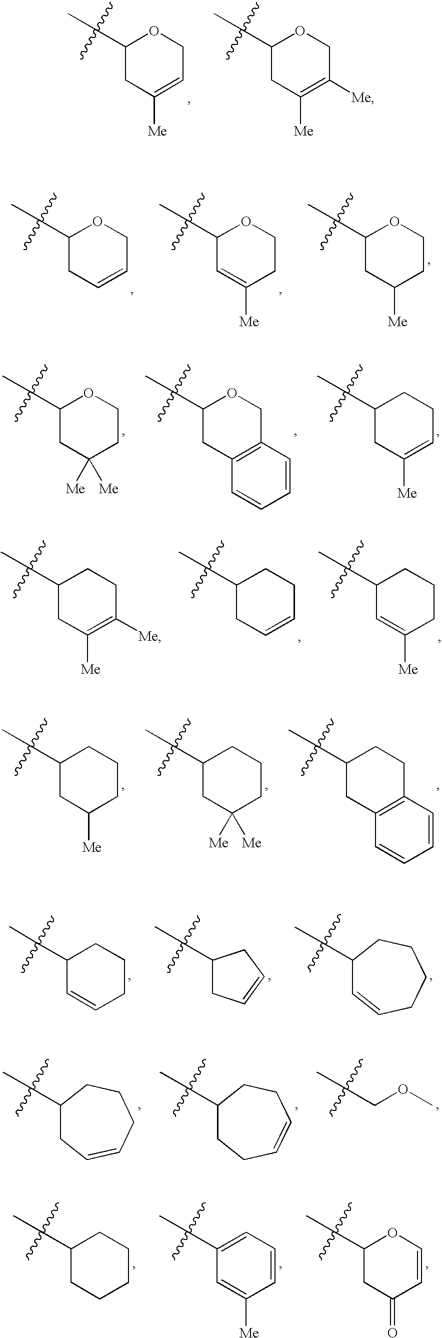

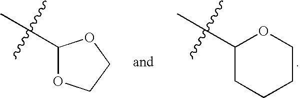

31. The process of claim 29, wherein the cross-metathesis catalyst is selected from a catalyst containing Ni, W, Ru or Mo metal.

32. The process of claim 31, wherein the cross-metathesis catalyst contains Ru.

33. The process of claim 32, wherein the cross-metathesis catalyst is selected from:
dichloro(phenylmethylene)bis(tricyclohexylphosphine)ruthenium(II),
[1,3-bis-(2,4,6-trimethylphenyl)-2-imidazolidinylidene]dichloro(phenylmethylene)(tricyclohexylphosphine)ruthenium(II),
dichloro[[2-(1-methylethoxy)phenyl]methylene](tricyclohexylphosphine)ruthenium(II),
[1,3-bis(2,4,6-trimethylphenyl)-2-imidazolidinylidene]dichloro[[2-(1-methylethoxy)phenyl]methylene]ruthenium(II),
[1,3-bis(2-methylphenyl)-2-imidazolidinylidene]dichloro[[2-(1-methylethoxy)phenyl]methylene]ruthenium(II),
[1,3-bis(2,4,6-trimethylphenyl)-2-imidazolidinylidene]dichloro[3-(2-pyridinyl-κN)propylidene-κC]ruthenium (II),
[1,3-bis(2-methylphenyl)-2-imidazolidinylidene]dichloro(benzylidene)(tricyclohexylphosphine)ruthenium (II),
dichloro(3-methyl-2-butenylidene)bis(tricyclohexylphosphine)ruthenium(II),
[1,3-bis(2,4,6-trimethylphenyl)-2-imidazolidinylidene]dichloro(3-methyl-2-butenylidene)(tricyclohexylphosphine)ruthenium(II),
[1,3-bis(2,4,6-trimethylphenyl)-2-imidazolidinylidene]dichloro(benzylidene)bis(3-bromopyridine)ruthenium(II).

34. The process of claim 29, wherein the reaction is conducted at temperature of about 15° C. to about 35° C.

35. The process of claim 29, wherein the reaction is conducted at a temperature of about 20° C. to about 150° C. with thermal heating.

36. The process of claim 29, wherein the reaction is conducted at a temperature of about 20° C. to about 150° C. with microwave irradiation heating.

37. The process of claim 29, wherein the amount of cross-metathesis catalyst is between about 1 mol % and about 40 mol %.

38. The process of claim 29 where R⁴ is CH₂OCH₃ and wherein processing the product comprises:
a. removing the methoxymethyl group of R⁴; and
b. epoxidizing an alkene at C16-C17.

39. The process of claim 29 where R⁴ is H and wherein processing the product comprises epoxidizing an alkene at C16-C17.

40. A method of treating a proliferative disease comprising administering a therapeutically effective amount of a compound of claim 1 or a pharmaceutically acceptable solvate, pharmaceutically acceptable salt, or pharmaceutically acceptable prodrug thereof.

41. A method of treating a proliferative disease comprising administering a therapeutically effective amount of a compound produced by the process of claim 9.

42. A method of treating a proliferative disease comprising administering a therapeutically effective amount of a compound produced by the process of claim 18.

43. A method of treating a proliferative disease comprising administering a therapeutically effective amount of a compound produced by the process of claim 29.

44. The method of claims 40-44 wherein the proliferative disease is cancer.

45. The method of claim 44, wherein the cancer is leukemia or myeloproliferative disorder.

46. A method of treating inflammatory disorders comprising administering a therapeutically effective amount of a compound of claim 1 or a pharmaceutically acceptable solvate, pharmaceutically acceptable salt, or pharmaceutically acceptable prodrug thereof.

47. A method of treating inflammatory disorders comprising administering a therapeutically effective amount of a compound produced by the process of claims 9, 18, or 29.

48. The method of claim 47 wherein the inflammatory disorder is psoriasis, eczema, multiple sclerosis, and arthritis.
